(19) United States
(12) Patent Application Publication
(10) Pub. No.: US 2006/0171922 A1
(43) Pub. Date: Aug. 3, 2006

(54) Helper virus-free herpesvirus amplicon particles and uses thereof

(76) Inventors: Howard J Federoff, Rochester, NY (US); William I Bowers, Webster, NY (US)

Correspondence Address: FISH & RICHARDSON PC, P.O. BOX 1022, MINNEAPOLIS, MN, 55440-1022, US

(21) Appl. No.: 10/516,211

(22) PCT Filed: May 30, 2003
(86) PCT No.: PCT/US03/17318

§ 371 Date: Dec. 19, 2005

Related U.S. Application Data
(60) Provisional application No. 60/385,230, filed on May 31, 2002.

(51) Int. Cl. A61K 48/00; C12N 15/869

(52) U.S. Cl. 424/93.2; 435/456; 514/44

(57) ABSTRACT

The invention features new helper virus-free methods for making herpes virus amplicon particles that can be used in immunotherapies, including those for treating any number of infectious diseases and cancers (including chronic lymphocytic leukemia, other cancers in which blood cells become malignant, lymphomas (e.g. Hodgkin's lymphoma or non-Hodgkin's type lymphomas). Described herein are methods of making helper virus-free HSV amplicon particles; cells that contain those particles (e.g., packaging cell lines or patient's cells, infected in vivo or ex vivo); particles produced according to those methods; and methods of treating a patient with an hf-HSV particle made according to those methods.

### CROSS-REFERENCE TO RELATED APPLICATIONS

This application claims priority from U.S. Provisional Patent Application Ser. No. 60/385,230, filed on May 31, 2002, which is incorporated herein by reference in its entirety.

### STATEMENT REGARDING GOVERNMENT SUPPORT

The work described herein was funded, in part, by grants from the National Institutes of Health. The government may, therefore, have certain rights in the invention.

### FIELD OF THE INVENTION

The present invention relates to methods for making helper virus-free preparations of herpes virus amplicon particles; the particles per se; and methods of using the particles to express proteins in cells.

### BACKGROUND

Herpes simplex virus (HSV) is a DNA virus capable of rapidly and efficiently infecting a wide variety of cell types (Leib and Olivo, BioEssays 15:547-554, 1993). Plasmid-based viral vectors derived from HSV, termed amplicons, are easy to construct and package into viral particles.

### SUMMARY

The compositions and methods of the present invention are based on a number of discoveries, including the discoveries that: (1) cells transduced with HSV amplicon vectors can process proteins encoded by the vectors for class I MHC presentation; (2) when used to deliver a viral antigen, herpes virus-based amplicon vectors can induce a cell-mediated immune response that is at least equivalent to that induced by live herpes virus vectors and that exceeds that induced by a modified vaccinia Ankara (MVA) vector; (3) animals immunized with HSV amplicon-transduced dendritic cells respond by producing antigen-specific cytotoxic T lymphocytes (e.g., animals immunized with an HSV-gp120 amplicon display a cell-mediated immune response); (4) animals infected with HSV-gp120 also exhibit a humoral immune response; (5) the expression of virion host shutoff (vhs) proteins in helper virus-free packaging systems improves amplicon titer, and vector stocks prepared in this way do not exhibit the pseudotransduction phenomenon (to further enhance packaging efficiency, an HSV transcriptional activator can be introduced into packaging cells); (6) helper virus-free amplicon preparations are superior to helper virus-containing amplicon preparations (see the studies below); (7) amplicon particles that express neurotrophin-3 can protect neurons from cisplatin-induced damage; and (8) including the Tc1-like Sleeping Beauty (SB) transposon system in the protocol to generate helper virus-free amplicon particles results in chromosomal integration of a transgene carried by the amplicon particle.

Accordingly, the invention features various methods for making helper virus-free herpes virus amplicon particles and for introducing nucleic acid sequences into cells (in vivo or in culture) using those particles. The particles of the invention, regardless of the precise method by which they are made, may be abbreviated herein as “hf-herpes virus amplicons” or “hf-HSV” particles. Any of these particles can be used in combination with a vector that expresses an enzyme (e.g., a transposase) that facilitates chromosomal integration of the transgene carried by the hf-HSV particles. Chromosomal integration can result in longer-term expression of the transgene. In either event (whether one uses an hf-herpes virus system to generate cells in which gene expression is altered by episomally- or chromosomally-integrated nucleic acid sequences), hf-herpes virus particles (or cells that contain them; whether those particles and cells are made by methods known in the art or by the new methods described below) can be administered to patients who have an infectious disease, cancer, a neurological deficit (including those in which neuron-specific proteins (e.g., neurotransmitters) are defective or underexpressed), or hearing loss. The invention encompasses new uses for known particles and cells as well as new particles and cells. The particles produced by the novel methods described below are different from those produced to date, even those produced by helper virus-free methods (they differ in their protein content and size; the present hf-HSV are less electron dense and are smaller in diameter). We describe the conditions amenable to treatment and the hf-HSV-based methods by which they can be treated in more detail after summarizing the methods for making the hf-HSV particles.

In one embodiment, the method may comprise or consist of generating a helper-free herpes virus amplicon particle (e.g., an hf-HSV) by: (1) providing a cell that has been stably transfected with a nucleic acid sequence that encodes an accessory protein (alternatively, a transiently transfected cell can be provided); and (2) transfecting the cell with (a) one or more packaging vectors that, individually or collectively, encode one or more (and up to all) HSV structural proteins but do not encode a functional herpes virus cleavage/packaging site and (b) an amplicon plasmid comprising a sequence that encodes a functional herpes virus cleavage/packaging site and a herpes virus origin of DNA replication. The amplicon plasmid described in (b) can also include a sequence that encodes a therapeutic agent. In another embodiment, the method may comprise or consist of cotransfecting a host cell with (a) an amplicon plasmid comprising an HSV origin of replication, an HSV cleavage/packaging signal, and a heterologous transgene expressible in the host cell, (b) one or more packaging vectors that, individually or collectively, encode all essential HSV genes but exclude all cleavage/packaging signals, (c) a vector encoding an accessory protein, and (d) an integration vector, wherein the integration vector encodes an enzyme that catalyzes a reaction within the cell, the consequence of the reaction being that the transgene carried by the amplicon plasmid is inserted into the genome of the cell. In yet another embodiment, the method may comprise or consist of transfecting a cell with (a) one or more packaging vectors that, individually or collectively, encode one or more HSV structural proteins (e.g., all HSV structural proteins) but do not encode a functional herpes virus cleavage/packaging site; (b) an amplicon plasmid comprising a sequence that encodes a functional herpes virus cleavage/packaging site, a herpes virus origin of DNA replication, and a sequence or transgene that encodes such products as an immunomodulatory protein (e.g., an immunostimulatory protein), a tumor-specific antigen, an antigen of an infectious agent, or a therapeutic agent (e.g., a growth factor); and (c) a nucleic acid sequence that encodes an accessory protein. These methods can also include an integration vector encoding an enzyme that catalyzes a reaction with the cell, the consequence of the reaction being that the transgene carried by the amplicon plasmid is inserted into the genome of the cell. In addition, these methods can include maintaining the cell under conditions that permit the cell to produce the herpes virus amplicon particle and, optionally, substantially isolating the herpes virus amplicon particle from the cell.

In either of these methods (or any method described herein), one or more of the following additional limitations may apply. For example, the herpes virus can be any of the more than 100 known species of herpes virus. For example, the herpes virus can be an alpha herpes virus (e.g., a Varicella-Zoster virus, a pseudorabies virus, or a herpes simplex virus (e.g., type 1 or type 2 HSV) or an Epstein-Barr virus. Similarly, the methods require sequences that encode an accessory protein, which can be a protein that inhibits the expression of a gene in the cell. For example, the accessory protein can be a virion host shutoff (vhs) protein (e.g., an HSV-1 vhs protein, an HSV-2 vhs protein, an HSV-3 vhs protein, bovine herpes virus 1 vhs protein, bovine herpes virus 1.1 vhs protein, gallid herpes virus 1 vhs protein, gallid herpes virus 2 virion hsp, suid herpes virus 1 vhs protein, baboon herpes virus 2 vhs protein, pseudorabies vhs protein, cercopithecine herpes virus 7 vhs protein, meleagrid herpes virus 1 vhs protein, equine herpes virus 1 vhs protein, or equine herpes virus 4 vhs protein). Any of these proteins can be operatively coupled to its native transcriptional control element(s) or to an artificial control element (i.e., a control element that does not normally regulate its expression in vivo).

The methods by which herpes virus amplicon particles are generated can also include a step in which the cell is transfected with a sequence encoding a VP16 protein, which may be transiently or stably expressed. Alternatively, or in addition, one can engineer a transcriptional activator to mimic VP16 (e.g., a pseudo-activator that recognizes cis elements but uses a different transcriptional activation domain). The VP16 protein can be HSV1 VP16, HSV-2 VP16, bovine herpes virus 1 VP16, bovine herpes virus 1.1 VP16, gallid herpes virus 1 VP16, gallid herpes virus 2 VP16, meleagrid herpes virus 1 VP16, or equine herpes virus 4 VP16.

The vhs and VP16 encoding sequences can be introduced into a cell on the same vector or on two different vectors or on two different types of vectors (e.g., both sequences can be introduced in the same plasmid, in two different plasmids, or in a plasmid and cosmid; this scenario is generally applicable in that the invention features methods in which more than one vector is used to introduce a component of the amplicon system into a host cell and there is no requirement that all of the vectors be of the same type). Sequences encoding vhs and/or VP16 can be transiently or stably introduced into cells (these methods are routine in the art), and the invention features a cell that is transiently or stably transfected with one or both of the sequences that encode one or more of a vhs or VP 16 protein.

As noted above, the herpes virus (e.g., HSV) amplicon particles are made by methods that employ one or more packaging vectors, which may comprise a cosmid (and may include a set of cosmids), a yeast artificial chromosome, a bacterial artificial chromosome, a human artificial chromosome, or an F element plasmid. A single packaging vector can encode the entire genome of a herpes virus, or the genome may be divided between two or more vectors (of the same type or of different types). For example, the packaging vectors can include a set of cosmids (e.g., a set of cosmids comprising cos6Δa, cos28, cos14, cos56, and cos48Δa). One or more packaging vectors, individually or collectively, can express the structural herpes virus proteins. The herpes virus origin of DNA replication is not present in the one or more packaging vectors.

In the method first described above (the method that employs a transiently or stably transfected cell), and as noted above, the amplicon plasmid can also include a sequence encoding a therapeutic agent (the sequence can also be referred to as a transgene) and, optionally, a regulatory sequence (e.g., a promoter) to increase the efficiency of expression of the therapeutic agent. The therapeutic agent can be a protein or an RNA molecule (e.g., an antisense RNA molecule, siRNA, or a ribozyme). In the event the therapeutic agent is a protein, the protein can be a receptor (e.g., a receptor for a growth factor or neurotransmitter), a signaling molecule (e.g., a growth factor or neurotransmitter), a transcription factor, a factor that promotes or inhibits apoptosis, a DNA replication factor, an enzyme, a structural protein, a neural protein (i.e., a protein expressed or differentially expressed in neurons), or a histone. The protein can also be an immunomodulatory protein (e.g., a cytokine, such as an interleukin, an interferon, or a chemokine, or a costimulatory molecule, such as a B7 molecule or CD40L), a tumor-specific antigen (e.g., PSA), or an antigen of an infectious agent (e.g., a virus such as a human immunodeficiency virus, a herpes virus, a papillomavirus, an influenza virus, or Ebola virus, a bacterium (e.g., an Escherichia (e.g., E. coli) Staphylococcus, Campylobacter (e.g., C. jejuni), Listeria (e.g., L. monocytogenes), Salmonella, Shigella or Bacillus (e.g., B. anthracis), or a parasite (e.g., parasites, the organisms that spread them, and the diseases they cause include Acetodextra sp., Allochanthochasmus sp., African sleeping sickness (African trypanosomiasis), Amblyomma americanum (lone star tick), American trypanosomiasis (Chagas' Disease), Allocreadium sp., Alloglossidium sp., American cockroach (Periplaneta americanus), Amoebiasis (Entamoeba histolytica), “Anchor worm” (Lernea sp.), Ancylostoma spp. (hookworms), Angiostrongylus cantonensis, Anisakis sp., Anopheles sp., Apocreadium sp., Apophallus sp., Argulus sp., Arthrocephalus (=Placoconus) sp., Ascaris sp. (human and pig roundworms), Aspidogaster sp., Auridistomum sp., Babesia bigemina (babesiosis), Balantidium coli, Baylisascaris procyonis, Bedbugs (Cimex spp.), Bilharziasis (schistosomiasis), Black-legged tick (Ixodes scarpularis), “Black spot” in fish (Uvulifer ambloplitis), Body louse (Pediculus humanus), Boophilus microplus (southern cattle tick), Bot(s) (bot fly), Bothriocepalus sp., Brugia malayi (brugian filariasis), Camallanus sp., Capillaria hepatica, Capillaria philippinenisis, Cattle tick (Boophilus microplus), Cephalogonimus sp., Cercarial dermatitis, Chagas' Disease (American trypanosomiasis), Chigger (Tunga penetrans), Chigoe (Tunga penetrans), Chilomastix mesnili (a commensal), Chique (Tunga penetrans), Choanotaenia sp., Cimex spp. (bedbugs), Clonorchis sinensis (Chinese/Oriental liver fluke), Cockroach, American (Periplaneta americanus), Coccidiosis (Eimeria and Isospora), Conspicuum sp., Cooperia spp., Corallobothrium sp., Cosmocerella sp., Cotylaspis sp., Cotylurus sp., Crab louse (Phthirus pubis), Crepidostomum sp., Cryptobia salmositica, Cryptosporidium parvum (cryptosporidiosis), Ctenocephalides sp. (fleas), Cutaneous larval migrans (CLM), Cuterebids (bot flies), Cyclospora cayetanesis, Cysticercosis, Deer flies (Tabanus sp.), Deer tick (Ixodes scarpularis), Dehli boil, Demodectic mange, Demodex sp. (follicle mites), Dermacentor sp. (dog tick), Dicrocoelium dendriticum (lancet fluke), Dictyangium sp., Dientamoeba fragilis, Dioctophyme renale, Diphyllobothrium latum, Diplogonoporus grandis, Diplostomulum sp., Dipylidium caninum (cucumber tapeworm), Dirofilaria immitis (canine heartworm), Dog tick (Dermacentor sp.), Dracunculiasis, Dracunculus medinensis, Dum-Dum fever, Echinococcus granulosus, Echinococcus multilocularis (hydatid disease), Echinorhynchus sp., Echinostoma spp., Eimeria sp. (coccidiosis), Elephantiasis (filariasis), Endolimax nana (a commensal), Entamoeba coli (a commensal), Entamoeba histolytica (amoebiasis, dysentery), Enterobius vermicularis (pinworms), Eosinophilic meningoencephalitis, Angiostrongylus cantonensis, Epistylis sp., Ergasilus sp., Espundia, Eurytrema pancreaticum, Eustrongylides sp., Face mange (Notoedres cati), Fasciola hepatica (sheep liver fluke), Fascioloides magna, Fasciolopsis buski, Fiery serpent (Dracunculus medinensis), Filariasis (elephantiasis), Fleas (Ctenocephalides sp.), Follicle mites (Demodex spp.), Giardia lamblia (giardiasis), Glaridacris catostomus, Glossina sp. (tsetse or tsetse fly), Gordius sp. (horsehair worms), Gregarina sp., Guinea worm (Dracunculus medinensis), Gyrocotyle sp., Gyrodactylus Sp., Haematoloechus medioplexus (frog lung fluke), Haemonchus spp., Haplobothrium sp., Heartworm (Dirofilaria immitis), Hemogregarina sp., Heterophyes heterophyes, Hookworms (Ancylostoma and Necator), Horse flies (Tabanus sp.), Horsehair worms (Nematomorpha), Hydatid disease (hydatidosis), Hymenolepis spp., Hymenolepis diminuta, Hymenolepis nana (Vampirolepis nana), Ichthyophthirius multifiliis (“ick” in fish), Iodamoeba butschlii (a commensal), Isospora sp. (coccidosis), Isospora belli, Ixodes scarpularis (Black-legged or deer tick), Jericho boil, Jigger (Tunga penetrans), Kala-azar, Leishmania spp. (leishmaniasis), Leptorhynchoides sp., Lernea sp. (“anchor worm”), Leucochloridium sp., Lice (body and pubic), Ligula intestinalis, Lissorchis sp., Loa loa, Lone star tick (Amblyomma americanum), Loxogenes sp., Lutztrema sp., Macracanthorhynchus hirudinaceus, Malaria (Plasmodium spp.), Mange, Megalodiscus temperatus, Menigoencephalitis, Angiostrongylus cantonensis, Mesocestoides sp., Metagonimus yokogawai, Metorchis conjunctus, Microcotyle sp., Microphallus sp., Moniezia expansa, Moniliformis sp., Multiceps serialis (Taenia serialis), Myxobolus (“whirling disease”), Necator americanus (hookworms), Nematodirus spp., Nematomorpha (horsehair worms), Notoedres cati, Notocotylus notocotylus, Obeliscoides cuniculi, Octomacrum sp., Onchocerca volvulus (onchocerciasis, riverblindness), Ophiotaenia sp., Ornithodorus turicata, Ostertagia spp., Panstrongylus megistus, Parabascus sp., Paragonimus westermani (human lung fluke), Pediculus humanus (body louse), Periplaneta americanus (American cockroach), Philometra sp., Pinworms (Enterobius vermicularis), Placobdella sp., Placoconus sp., Plagiorhynchus sp., Plasmodium spp. (malaria), Platynostomum sp., Pleorchis sp., Polymorphus minutus, Pomphorhynchus sp., Polystoma sp., Polystomoides sp., Postharmostomum helices, Prosthogonimus macrorchis, Proteocephalus sp., Proterometra sp., Phthirus pubis (pubic or crab louse), Pubic louse (Phthirus pubis), Rajonchocotyle sp., Red mange (canine demodetic mange), Relapsing fever tick (Ornithordus turicata), Rhipidocotyle sp., Rhodnius prolixus, Rhopalias sp., Riverblindness (onchocerciasis), Sand flea (Tunga penetrans), Sarcocystis spp., Sarcoptes scabiei sp. (sarcoptic mange), Sarcoptic mange, Schistosoma sp. (schistosomiasis, blood flukes), Schistosome cercarial dermatitis, Southern cattle tick (Boophilus microplus), Sparganosis, Spinitectus sp., Strongyloides stercoralis, Styphlodora sp., Swimmer's itch, Tabanus sp. (horse or deer flies), Taenia spp. (beef and pork tapeworms), Taenia pisiformis, Taenia serialis, Telorchis sp., Temnocephala sp., Tenebrio molitor, Tetraonchus sp., Tetraphyllidean cestodes, Toxocara canis (canine roundworm), Toxoplasma gondii (toxoplasmosis), Triaenophorus crassus, Triatoma infestans, Tribolium confusum (confused flour beetle), Trichinella spiralis (trichinosis), Trichodina sp., Trichomonas vaginalis (trichomoniasis), Trichostrongylus spp., Trichuris spp. (whipworms), Triganodistomum sp., Trypanorhynchid cestodes, Trypanosoma cruzi (American trypanosomiasis, Chagas' Disease), Trypanosoma spp. (African trypanosomiasis, “sleeping sickness”), Tsetse or tsetse fly (Glossina sp.), Tunga penetrans, Urogonimus sp., Uta, Uvulifer ambloplitis (“black spot” in fish), Vampirolepis nana (Hymenolepis nana), Visceral larval migrans (VLM), Warble(s), Watsonius sp., Whipworms (Trichuris spp.), “Whirling disease” in fish (Myxobolus sp.), Wuchereria bancrofti (filariasis), Zonorchis sp., Zygocotyle lunata)).

In the third method described above, the amplicon plasmid can encode an immunomodulatory protein, a tumor-specific antigen, or the antigen of an infectious agent (including those described above). It will be apparent to one of ordinary skill in the art which therapeutic agents can be expressed to generate particles and cells useful for treating which conditions. For example, one would select an antigen expressed by HIV (e.g., gp120 or gag-pol) to treat a patient who is infected, or who may become infected, with HIV; one would select a prion protein to treat a patient who has, or who is at risk of developing, CJD; and so forth.

In another embodiment, the invention features a method that includes (a) co-transfecting a host cell with the following: (i) an amplicon vector comprising an HSV origin of replication, an HSV cleavage/packaging signal, and a heterologous (i.e., non-HSV) transgene expressible in a patient, (ii) one or more vectors that individually or collectively encode all essential HSV genes but exclude all cleavage/packaging signals, and (iii) a vhs expression vector encoding a virion host shutoff protein; and (b) isolating HSV amplicon particles produced by the host cell, the HSV amplicon particles including the transgene (see the PCT application published under number WO 0189304, which is incorporated herein by reference in its entirety). The components used in this method (enumerated as (i), (ii), and (iii) above) may be referred to herein as an “amplicon system.”

In other embodiments, the invention features methods of constructing a herpes virus amplicon (e.g., an HSV amplicon particle) that integrates into the chromosomes of dividing and non-dividing cells. The conventional amplicon genome is maintained as an episome and is not mitotically maintained during cell division. However, vectors made by the methods described herein can be used to transfer transgenes from parent cells to daughter cells. The methods can be carried out by combining a transposon-encoding system (e.g., the Tc1-like Sleeping Beauty (SB) transposon system) with the amplicon. When cells contain both an enzyme that mediates chromosomal integration and a corresponding amplicon particle bearing a heterologous transgene, the transgene can integrate into the genomes of both mitotically active and post-mitotic cell types.

To enhance amplicon titers, the methods of the invention can include introducing in trans a vector including a sequence that encodes a virion host shutoff protein. Co-transfection of this plasmid (e.g., a plasmid containing the vhs protein-encoding gene UL41) with the amplicon and packaging reagents can result in 10-fold higher amplicon titers and stocks that do not exhibit the pseudotransduction phenomenon. The HSV transcriptional activator VP16 can be introduced into packaging cells prior to the packaging components; pre-loading of packaging cells with VP16 can lead to an additional enhancement of amplicon titers.

In addition, the invention features kits containing one or more of the herpes virus amplicon particles described herein; one of more of the cells containing them; or one or more of the components useful in generating either the particles or the cells. For example, a kit can include a packaging vector and an amplicon plasmid. Optionally, the kit can also contain stably transfected cells. Optionally, the kit can include instructions for use, and any of the kits that contain one or more components of the amplicon system (e.g., the components enumerated above by (i), (ii), and (iii)) can also contain a vector that encodes an enzyme that mediates integration of the transgene carried by the amplicon particle into the genome of a host cell.

The particles generated by the methods of the invention, cells that contain those particles, and the components used to generate them (e.g. the components enumerated above by (i), (ii), and (iii); packaging cell lines; or patients' cells, infected in vivo or ex vivo) are also within the scope of the invention. The particles and cells that come within the scope of the invention include any of those made using the methods described herein. The cell can be virtually any differentiated cell or a precursor thereof. For example, the cell can be a neuron, a blood cell, a hepatocyte, a keratinocyte, a melanocyte, a neuron, a glial cell, an endocrine cell, an epithelial cell, a muscle cell, a prostate cell, or a testicular cell. The cell can also be a malignant cell (including any of those that arise from the differentiated cells just listed; e.g. a neuroblastoma, a lymphoma or leukemia cell, a hepatocarcinoma cell etc.). Alternatively, or in addition, the cell can be any cell that is infected with an infectious agent (including a virus, a bacterium, a parasite, or a prion including, but not limited to, those types described herein).

hf-herpes virus particles (e.g., hg-HSV particles), regardless of the precise method by which they are made, can contain one or more genes encoding one or more therapeutic proteins (full-length or biologically active or therapeutically effective fragments or mutants thereof), and they can be used to transduce cells, including those that contain an infectious agent. The term “infectious agent,” as used herein, encompasses viruses, bacteria, mycobacteria, parasites, and prions unless a specific exception is explicitly noted in the description below; a cell that contains an infectious agent may be referred to herein as an infected cell (and may be a cell from a human, cow, sheep, or other animal; while the compositions and methods described herein can be administered to (or applied to) humans, they can also be administered to (or applied to) domesticated animals or livestock). As noted above, the patient can have any one of a wide variety of infectious diseases, including those associated with non-conventional infectious agents, such as prions (e.g., a transmissible spongiform encephalopathy (TSE) such as Creutzfeld-Jacob disease (CJD) or Gertsmann-Straussler-Scheinker syndrome (GSS) in man) and/or any one of a wide variety of cancers (including chronic lymphocytic leukemia, other cancers in which blood cells become malignant, and lymphomas (e.g. Hodgkin's lymphoma or non-Hodgkin's type lymphomas), a melanoma, a glioblastoma, an astrocytoma, a pancreatic cancer, a cancer of the reproductive system, a cancer of the endocrine system, a neuroblastoma, a breast cancer, a colorectal cancer, a stomach cancer, a cancer of the throat or within or around the mouth, a lung cancer, or a bladder cancer). Other conditions amenable to treatment include neurological disorders (e.g., Alzheimer's Disease and Parkinson's Disease; additional exemplary conditions are disclosed below) and disorders that result in partial or complete loss of hearing (including loss with age).

HSV amplicon particles have been used to express neuroprotective or neuroregenerative factors at high levels in various disease settings. Disease targets related to hearing loss have proven especially amenable to HSV-directed gene transfer. In the context of age-related hearing loss (presbycusis) and ototoxic drug-induced hearing loss (e.g., hearing loss following administration of aminoglycosides or cisplatin), HSV amplicon particles that express the neurotrophic factor NT-3 have provided protection against spiral ganglion neuron (SGN) degeneration. Accordingly, one can treat a patient who has, or who is likely to have, some hearing loss by administering hf-HSV particles that express neurotrophic factors before, during, or after a patient has been exposed to an agent (e.g., a chemotherapeutic agent) that adversely affects cells within the auditory system (e.g., SGNs).

The therapeutic protein expressed by the particles can be an immunostimulatory protein and may be a neoantigen (e.g., a tumor-specific antigen, such as prostate-specific antigen (PSA)). For example, the immunostimulatory protein can be an antigen associated with (e.g., expressed by) an infectious agent such as a prion protein or a non-infectious mutant or fragment thereof. The immunostimulatory protein can also be a particular viral antigen or an antigenic fragment thereof (e.g., the immunostimulatory protein can be tat, nef, gag/pol, vp, or env from an immunodeficiency virus such as HIV-1 or HIV-2) or a particular bacterial, mycobacterial, or parasitic antigen or an antigenic fragment thereof. For example, the therapeutic protein can be a portion of Prp^{c} (the non-infectious normal cellular prion protein) (e.g., residues 76-112; 134-160; 150-177; or 198-228 of SEQ ID NO:______; see also FIG. 14; additional prion sequences are known by, and available to, those of ordinary skill in the art and can also be used as described herein). Alternatively, or in addition, the hf-HSV particles of the invention can be used to express single-chain variable regions of antibodies (scFv), including those specific to Prp^{sc} (infectious prion agents). Similarly, single chain antibodies (which can be humanized by methods known in the art) that are directed against pathogenic antigens can be administered to patients who have been, or who may be, infected with or exposed to those agents. Expression of single-chain variable regions can be used to treat other conditions (e.g., cancer and neurological disorders) as well. For example, variable regions that specifically bind Aβ and α-synuclein can be used to treat patients who have, or who may develop, Alzheimer's Disease or Parkinson's Disease, respectively.

In one embodiment, an affected cell (e.g., an infected cell, a malignant cell, or one affected by neurological disease) is transduced (in vivo or ex vivo) with an hf-HSV amplicon particle that encodes an immunostimulatory protein (i.e., any protein or peptide that, when expressed by a target cell, induces or enhances an immune response to that cell). For example, a patient who has cancer can be treated with an HSV amplicon particle (or a cell within which it is contained) that expresses an antigen and a polypeptide that acts as a general stimulator of the immune system or a specific protein, such as a tumor-specific antigen (e.g., prostate-specific antigen (PSA)) (these particles and cells can be those made by the methods described herein). Similarly, a patient who has an infectious disease can be treated with an HSV amplicon particle (or a cell within which it is contained) that expresses an antigen and a polypeptide that acts as a general stimulator of the immune system or a specific antigen associated with (i.e., expressed by) the infectious agent (here again, the patients that are treated for an infectious disease can be treated with particles or cells made by the methods described herein). Polypeptides that act as general stimulators of the immune system include cytokines, including chemotactic cytokines (also known as chemokines) and interleukins, adhesion molecules (e.g., I-CAM) and costimulatory factors necessary for activation of B cells or T cells.

More generally, the methods of the invention including treating patients (such as those described above) by (a) providing an HSV amplicon particle that includes at least one transgene that encodes a therapeutic product and (b) exposing cells of the patient (e.g., infected cells, malignant cells, or neural or pre-neural cells) to the HSV amplicon particles under conditions effective for infective transformation of the cells. The therapeutic transgene product is expressed in the cells (e.g., in vivo) and thereby. delivers a therapeutically effective amount of the therapeutic product to the patient. Physicians and others of ordinary skill in the art are well able to determine whether an agent is therapeutically effective. They can, for example, observe an improvement in an objective sign of disease (e.g., an improvement in cognitive skills, motor skills, memory, platelet count, reduction of fever, or reduction of tumor size). An agent is also therapeutically effective when a patient reports an improvement in a subjective symptom (e.g. less fatigue).

Gene therapy vectors based on the herpes simplex virus have a number of features that make them advantageous in clinical therapies. They exhibit a broad cellular tropism, they have the capacity to package large amounts of genetic material (and thus can be used to express multiple genes or gene sequences), they have a high transduction efficiency, and they are maintained episomally, which makes them less prone to insertional mutagenesis. In addition to infecting many different types of cells, HSV vectors can transduce non-replicating or slowly replicating cells, which has therapeutic advantages. For example, freshly isolated cells can be transduced in tissue culture, where conditions may not be conducive to cell replication. The ability of HSV vectors to infect non-replicating or poorly replicating cells also means that cells (such as tumor cells) that have been irradiated can still be successfully treated with HSV vectors.

The transduction procedure can also be carried out fairly quickly, freshly harvested human tumors have been successfully transduced within about 20 minutes. As a result, cells (such a tumor cells) can be removed from a patient, treated, and readministered to the patient in the course of a single operative procedure (one would readminister tumor cells following transduction with, for example, an immunostimulatory agent (HSV vectors encoding immunomodulatory proteins and cells transduced with such vectors can confer specific antitumor immunity that protects against tumor growth in vivo).

On the other hand, it is inherently difficult to manipulate a large viral genome (150 kb) and HSV-encoded regulatory and structural viral proteins may be toxic (Frenkel et al., Gene Ther. 1 Suppl. 1:S40-46, 1994).

Additionally, the invention features any of the HSV amplicon particles mentioned above as a medicament. Such a medicament may be for use in treating a patient who has cancer, or who may develop cancer, in which the therapeutic protein is an immunomodulatory protein or a tumor-specific antigen. A medicament may be for use in treating a patient who has a prion-associated disease (e.g., Creutzfeld-Jacob Disease). Or, a medicament may be for use in treating a patient who has, or who is at risk for, hearing loss; this can include a method in which the transgene encodes a neurotrophin (e.g., neurotrophin-3). Other medicaments for use in treating or preventing other diseases, disorders, or conditions are also contemplated in this invention.

The invention also features compositions for use as medicaments in treating a patient who has, or who is at risk for, hearing loss, in which the compositions comprise or consist of (a) an amplicon plasmid comprising an HSV origin of replication, an HSV cleavage/packaging signal, and a heterologous transgene expressible in the host cell, (b) one or more vectors that, individually or collectively, encode all essential HSV genes but exclude all cleavage/packaging signals, and (c) a vector encoding an accessory protein, wherein the transgene encodes a therapeutic protein (e.g., neurotrophin (e.g., neurotrophin-3)) that exerts a protective effect on spiral ganglion neurons.

The invention also includes use of any of the HSV amplicon particle of the invention for the manufacture of medicaments. Such medicaments may be for use in treating a patient who has cancer, or who may develop cancer (e.g., in which the therapeutic protein is an immunomodulatory protein or a tumor-specific antigen). They may be for the manufacture of a medicament for use in treating a patient who has a prion-associated disease (e.g., Creutzfeld-Jacob Disease). Or, they may be for the manufacture of a medicament for use in treating a patient who has, or who is at risk for, hearing loss (e.g., in which the transgene encodes a neurotrophin (e.g., neurotrophin-3)).

In addition, the invention encompassed use of compositions for the manufacture of a medicament for use in treating a patient who has, or who is at risk for, hearing loss, in which such compositions comprise or consist of (a) an amplicon plasmid comprising an HSV origin of replication, an HSV cleavage/packaging signal, and a heterologous transgene expressible in the host cell, (b) one or more vectors that, individually or collectively, encode all essential HSV genes but exclude all cleavage/packaging signals, and (c) a vector encoding an accessory protein, in which the transgene encodes a therapeutic protein (e.g., neurotrophin (e.g., neurotrophin-3) that exerts a protective effect on spiral ganglion neurons.

In addition to the particular methods, compositions, and uses described above, the invention also includes combinations and permutations of these methods, compositions, and uses.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. All publications, patent applications, patents, and other references cited herein are incorporated by reference in their entirety. In case of conflicting subject matter, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a panel of four photomicrographs. Murine dendrite cells were photographed using phase contrast optics and fluorescent light after infection with HSV-creGFP or HSV-OVA amplicons (MOI=1).

FIG. 2 is a schematic representation of an infection procedure and photographs of activated T cells following co-culture with infected dendritic cells.

FIG. 3 is a schematic representation of an immunization and line graphs of the resulting cytotoxic T lymphocyte (CTL) response.

FIG. 4 is a bar graph representing the expression of IL-12 p70 (ng/ml) following treatment of dendritic cells (antigen presenting cells (APCs)) with one of two HSV amplicons (one that expresses PSA and one that expresses p35) followed by activation with oligonucleotides that contain an immunostimulatory CpG sequence or oligonucleotides in which the CpG sequence is altered to GpC.

FIG. 5 is a photograph of a Western blot. Lysates were prepared from HSVgp120-infected NIH 3T3 cells.

FIG. 6 is a series of four bar graphs illustrating the cellular responses to class I-restricted peptides from gp120.

FIG. 7 is a bar graph made by analyzing the humoral response in mice immunized with HSVgp120 (anti-env IgG responses in serum).

FIG. 8 is a graph plotting the results of a cell lysis assay (JAM). HSVgp120 mediated induction of CTL activity.

FIG. 9 is a series of four bar graphs illustrating the effect of administering an HSV-gp120 amplicon by three common routes of administration (intramuscular, subcutaneous, or intraperitoneal).

FIG. 10 is a Table of essential HSV-1 genes.

FIG. 11 shows three Tables. The uppermost concerns IL-2 production following transduction of CLL cells with helper virus-containing and helper virus-free amplicon stocks; the middle table concerns the % of CLL cells expressing B7.1 and CD40L following transduction with helper virus-containing and helper virus-free amplicon stocks; the lower table concerns gamma-interferon levels in supernatant derived from CTL assays using CLL cells transduced with helper virus-free amplicon stocks.

FIGS. 12A and 12B are schematic representations of suitable amplicon vectors. FIG. 12A represents the empty amplicon vector pHSVlac, which includes the HSV-1 a segment (cleavage/packaging or pac signal), the HSV-1 c region (origin of replication), an ampicillin resistance marker, and an E. coli lacZ marker under the control of HSV IE4 promoter and SV40 polyadenylation signal. FIG. 12B represents insertion of a transgene in a site (BamHI) adjacent to the HSV-1 a segment, forming pHSVlac/trans.

FIGS. 13A and 13B are schematic representations of the HSV-1 genome and the overlapping set of five cosmids C6Δa48Δa (cos6Δa, cos28, cos14, cos56, and cos48Δa (Fraefel et al., J. Virol. 70:7190-7197, 1996). In the HSV-1 genome of FIG. 13A, only the IE4 gene, oriS and oriL are shown. The a sequences, which contain the cleavage/packaging sites, are located at the junction between long and short segments and at both termini. In FIG. 13B, the deleted a sequences in cos6Δa and cos48Δa are indicated by “X”.

FIG. 14 is a representation of the amino acid sequence and nucleic acid sequence of a mouse prion protein (PRNP) gene (Westaway et al., Cell 51:651-662, 1987).

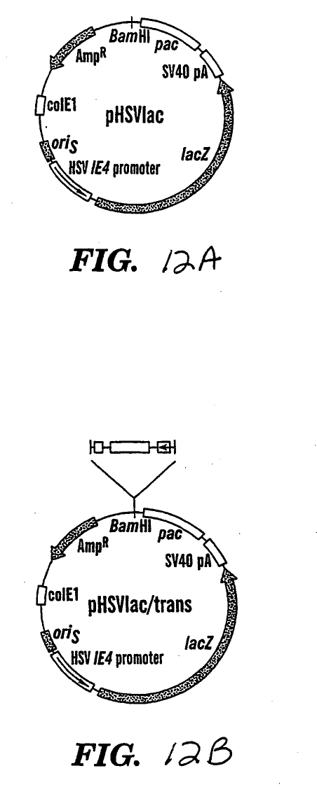

FIGS. 15A and 15B are photographs of RNA and protein blots, respectively, used to analyze NT-3myc transcripts and proteins in cochlear explant cultures transduced with hf-HSV amplicon particles. RT-PCR products were amplified from HSVnt-3myc-transduced P3 mouse cochlear explants using primers specific for the NT-3myc chimeric cDNA that gives rise to a 222-bp fragment (see further details in Example 13). The NT-3myc transcript was detected only in HSVnt-3myc-transduced cultures (FIG. 15A, lane 2, top) and was absent from HSVmiap- or mock-infected cultures (FIG. 15A, lanes 1 and 3, top). HPRT (used as a control) was amplified from all cultures (FIG. 15A, bottom). Protein lysates were prepared from HSVmiap—(FIG. 15B, lane 1), HSVnt-3myc—(FIG. 15B, lane 2), or mock-transduced (FIG. 15B, lane 3) cochlear explants. The myc-tagged NT-3 transgene was detected only in HSVnt-3myc-infected cultures.

FIG. 16 is a bar graph demonstrating the high levels of secreted NT-3 myc produced by HSVnt-3myc-transduced cochlear explants. Supernatants collected from cochlear cultures that were uninfected (“control”), or transduced with HSVmiap (“HSVmiap”) or HSVnt-3myc (“HSVnt-3myc) were analyzed using an NT-3-specific ELISA. The level of secreted NT-3 was 14 times higher in HSVnt-3myc-transduced cultures than in HSVmiap-infected and uninfected control groups. The data are represented as the mean supernatant concentrations of NT-3 (pg/ml; n=3; see also Example 13).

FIG. 17 is a bar graphs demonstrating the number of neurites in cochlear explants cultured in serum-free medium for 48 hours and left uninfected (“control”) or infected with HSVnt-3myc or HSVmiap. Immunocytochemistry specific to NF 200 was performed to visualize SNG somata and afferents. Explants infected with HSVnt-3myc exhibited enhanced number of neurites compared to HSVmiap-infected or uninfected control groups. The data are represented as the mean number of neurites per cochlear explant (n=3) (see also Example 13).

FIG. 18 is a bar graphs representing SGN survival (% control) in cochlear explants transfected with HSVmiap (open bars) or HSVnt-3myc (closed bars) and exposed to various concentrations of cisplatin (4, 6, or 8 μg/ml). Pretreatment with HSVnt3-myc substantially protected SGNs from cisplatin damage. The percentage of SGN survival from each treatment group was calculated as the number of NF 200-positive neurons in treated cultures divided by the number of NF 200-positive neurons detected in untreated control explant cultures multiplied by a factor of 100 (n=3) (see also Example 13).

FIG. 19 is a bar graph demonstrating integration of HSV amplicon-delivered Sleeping Beauty/T-βgeo transposon in BHK cells. Monolayers of BHK cells were left untreated or were transduced with 5×10⁴ virions of HSVsb alone, HSVT-βgeo alone, or HSVT-βgeo plus HSVsb. Three days later, cultures were placed under G418 selection, which was continued for two weeks to allow for colony growth. Resultant G418-resistant colonies were stained with X-gal and enumerated. Co-transduction of HSVT-βgeo and HSVsb led to a significant enhancement of drug-resistant colony formation, suggesting integration has occurred in the mitotically active BHK cells. The “*” indicates a statistically significant difference between HSVT-βgeo alone and HSVT-βgeo plus HSVsb treatment (p<0.05) (see also Example 15).

FIGS. 20A-20C are bar graphs demonstrating that co-transduction of primary neuronal cultures with HSVT-βgeo and HSVsb results in enhanced gene expression and high retention of transgenon DNA. Primary neuronal cultures established from E15 mouse embryos were transduced with HSVsb and/or HSVT-βgeo and analyzed at Days 4 or 9 post-transduction by enumeration of LacZ-positive cells (FIG. 20A), β-galactosidase activity (FIG. 20B) and quantitation of retained transgenon DNA sequences (FIG. 20C). The “*” indicates a statistically significant difference between HSVT-βgeo alone and HSVT-βgeo plus HSVsb combination group (p<0.05).

FIG. 21 is a schematic representation of a construct of the invention within the genome of a host cell. Primary neuronal cultures established from E15 mouse embryos were transduced with HSVsb and HSVT-βgeo and high molecular weight DNA harvested on Day 9 post-transduction. Inverse PCR was performed to determine novel flanking sequences of the integrated transgenon using a series of nested primers. Amplified products were isolated, cloned, and sequenced. Novel mouse-derived flanking sequences are shown.

FIGS. 22A-22C are bar graphs of various parameters measured after transduction with HSVsb and/or HSVT-βgeo. HSVsb and/or HSVT-βgeo were administered stereotactically to the striata of C57BL/6 mice and animals were sacrificed at 7, 21, and 90 days post-transduction. HSVPrPUC amplicon virions were included in the HSVsb only and HSVT-βgeo only groups to normalize viral particle input. Tissue blocks consisting of the striatal injection site were excised, homogenized, and analyzed initially for β-galactosidase reporter gene expression by the Galacto-Lite assay (FIG. 22A). Total genomic DNA was purified from these lysates and subjected to real-time quantitative PCR to detect either transgenon sequences (FIG. 22B) or sequences specific to the Sleeping Beauty-expressing amplicon vector (FIG. 22C). The “*” indicates a statistically significant difference between HSVT-βgeo alone and HSVT-βgeo plus HSVsb treatments (p<0.05).

### DETAILED DESCRIPTION

Helper virus-free systems for packaging herpes virus particles, including those described herein, include at least one vector (herein, “the packaging vector”) that, upon delivery to a cell that supports herpes virus replication, will form a DNA segment (or segments) capable of expressing sufficient structural herpes virus proteins that a herpes virus particle will assemble within the cell. When the particle assembles, amplicon plasmids that may also be present, can be packaged within the particle as well. In packaging systems that employ helper viruses, amplicon plasmids rely on the helper virus function to provide the replication machinery and structural proteins necessary for packaging amplicon plasmid DNA into viral particles. Helper packaging function is usually provided by a replication-defective virus that lacks an essential viral regulatory gene. The final product of helper virus-based packaging contains a mixture of varying proportions of helper and amplicon virions. Recently, helper virus-free amplicon packaging methods were developed by providing a packaging-deficient helper virus genome via a set of five overlapping cosmids (Fraefel et al., J. Virol. 70:7190-7197, 1996; see also U.S. Pat. No. 5,998,208) or by using a bacterial artificial chromosome (BAC) that encodes for the entire HSV genome minus its cognate cleavage/packaging signals (Stavropoulos and Strathdee, J. Virol. 72:7137-7143, 1998; Saeki et al., Hum Gene Ther. 9:2787-2794, 1998). Such cosmids can be used as the packaging vector in the methods described herein. Thus, the packaging vector can be a cosmid-based vector or a set of vectors including cosmid-based vectors that are prepared so that none of the viral particles used will contain a functional herpes virus cleavage-packaging site containing sequence. This sequence, which is not encoded by the packaging vector(s), is referred to as the “a” sequence. The “a” sequence can be deleted from the packaging vector(s) by any of a variety of techniques practiced by those of ordinary skill in the art. For example, one can simply delete the entire sequence (by, for example, the techniques described in U.S. Pat. No. 5,998,208). Alternatively, one can delete a sufficient portion of the sequence to render it incapable of packaging. Another alternative is to insert nucleotides into the site that render it non-functional.

The core of the herpes virus particle is formed from a variety of structural genes that create the capsid matrix. It is necessary to have those genes for matrix formation present in a susceptible cell used to prepare particles. Preferably, the necessary envelope proteins are also expressed. In addition, there are a number of other proteins present on the surface of a herpes virus particle. Some of these proteins help mediate viral entry into certain cells, and as this is known to those of ordinary skill in the art, one would know to alter the sequences expressed by the viral particle in order to alter the cell type the viral particle infects or improve the efficiency with which the particle infects a natural cellular target. Thus, the inclusion or exclusion of the functional genes encoding proteins that mediate viral entry into cells will depend upon the particular use of the particle.

In addition to a packaging vector, the herpes virus amplicon systems described herein include an amplicon plasmid. The amplicon plasmid contains a herpes virus cleavage/packaging site containing sequence, an origin of DNA replication (ori) that is recognized by the herpes virus DNA replication proteins and enzymes, and a transgene of interest (e.g., a nucleic acid sequence that encodes a therapeutically effective protein). This vector permits packaging of desired nucleotide inserts in the absence of helper viruses. In some embodiments, the amplicon plasmid contains at least one heterologous DNA sequence that is operatively linked to a promoter sequence (we discuss promoter and other regulatory sequences further below). More specifically, the amplicon plasmid can contain one or more of the following elements: (1) an HSV-derived origin of DNA replication (ori) and packaging sequence (“a” sequence); (2) a transcription unit driven typically by the HSV-1 immediate early (IE) 4/5 promoter followed by an SV-40 polyadenylation site; and (3) a bacterial origin of replication and an antibiotic resistance gene for propagation in E. coli (Frenkel, supra; Spaete and Frenkel, Cell 30:295-304, 1982).

Methods for Generating Helper Virus-Free Herpes Virus Amplicons

Generally, the methods of the invention are carried out by transfecting a host cell with several vectors and then isolating HSV amplicon particles produced by the host cell (while the language used herein may commonly refer to a cell, it will be understood by those of ordinary skill in the art that the methods can be practiced using populations (whether substantially pure or not) of cells or cell types, examples of which are provided elsewhere in our description). The method for producing an hf-HSV amplicon particle can be carried out, for example, by co-transfecting a host cell with: (i) an amplicon vector comprising an HSV origin of replication, an HSV cleavage/packaging signal, and a heterologous transgene expressible in a cell; (ii) one or more vectors that, individually or collectively, encode all essential HSV genes but exclude all cleavage/packaging signals; and (iii) a vhs expression vector encoding a virion host shutoff protein. One can then isolate or purify (although absolute purity is not required) the HSV amplicon particles produced by the host cell. When the HSV amplicon particles are harvested from the host cell medium, the amplicon particles are substantially pure (i.e., free of any other virion particles) and present at a concentration of greater than about 1×10⁶ particles per milliliter. To further enhance the use of the amplicon particles, the resulting stock can also be concentrated, which affords a stock of isolated HSV amplicon particles at a concentration of at least about 1×10⁷ particles per milliliter.

The amplicon vector can either be in the form of a set of vectors or a single bacterial-artificial chromosome (“BAC”), which is formed, for example, by combining the set of vectors to create a single, doublestranded vector. As noted above, methods for preparing and using a five cosmid set are disclosed in, for example, Fraefel et al. (J. Virol, 70:7190-7197, 1996), and methods for ligating the cosmids together to form a single BAC are disclosed in Stavropoulos and Strathdee (J. Virol. 72:7137-43, 1998). The BAC described in Stavropoulos and Strathdee includes a pac cassette inserted at a BamHI site located within the UL41 coding sequence, thereby disrupting expression of the HSV-1 virion host shutoff protein.

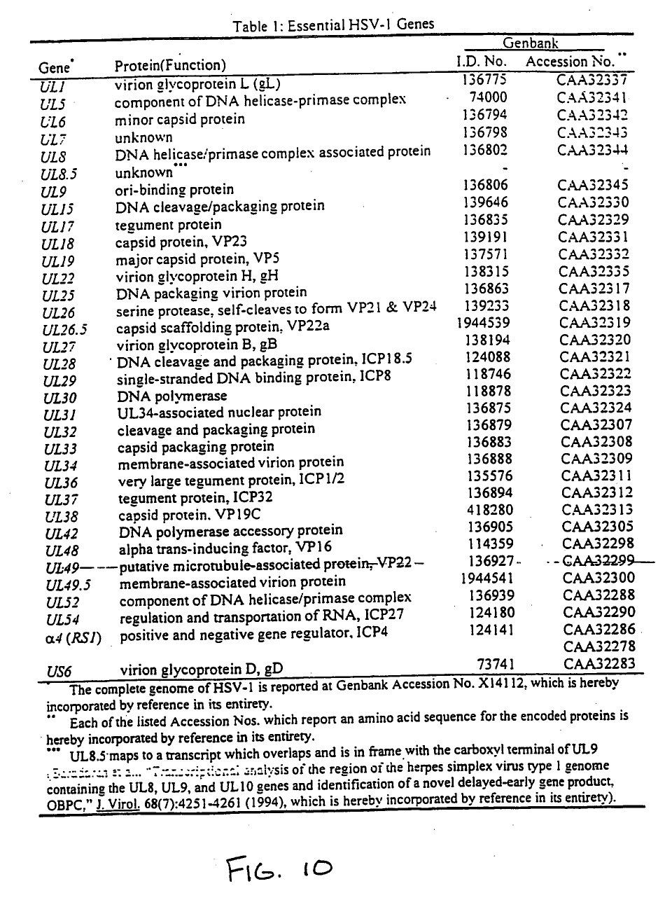

By “essential HSV genes”, it is intended that the one or more vectors include all genes that encode polypeptides that are necessary for replication of the amplicon vector and structural assembly of the amplicon particles. Thus, in the absence of such genes, the amplicon vector is not properly replicated and packaged within a capsid to form an amplicon particle capable of adsorption. Such “essential HSV genes” have previously been reported in review articles by Roizrnan (Proc. Natl. Acad. Sci. USA 11:307-113, 1996; Acta Viroloeica 43:75-80, 1999). Another source for identifying such essential genes is available at the Internet site operated by the Los Alamos National Laboratory, Bioscience Division, which reports the entire HSV-1 genome and includes a table identifying the essential HSV-1 genes. The genes currently identified as essential are listed in the Table provided as FIG. 10.

In other embodiments, a helper-free herpes virus amplicon particle (e.g., an hf-HSV) can be generated by: (1) providing a cell that has been stably transfected with a nucleic acid sequence that encodes an accessory protein (alternatively, a transiently transfected cell can be provided); and (2) transfecting the cell with (a) one or more packaging vectors that, individually or collectively, encode one or more (and up to all) HSV structural proteins but do not encode a functional herpes virus cleavage/packaging site and (b) an amplicon plasmid comprising a sequence that encodes a functional herpes virus cleavage/packaging site and a herpes virus origin of DNA replication (ori). The amplicon plasmid described in (b) can also include a sequence that encodes a therapeutic agent. In another embodiment, the method comprises transfecting a cell with (a) one or more packaging vectors that, individually or collectively, encode one or more HSV structural proteins (e.g., all HSV structural proteins) but do not encode a functional herpes virus cleavage/packaging site; (b) an amplicon plasmid comprising a sequence that encodes a functional herpes virus cleavage/packaging site, a herpes virus origin of DNA replication, and a sequence that encodes an immunomodulatory protein (e.g., an immunostimulatory protein), a tumor-specific antigen, an antigen of an infectious agent, or a therapeutic agent (e.g., a growth factor); and (c) a nucleic acid sequence that encodes an accessory protein.

The HSV cleavage/packaging signal can be any cleavage/packaging that packages the vector into a particle that is capable of adsorbing to a cell (the cell being the target for transformation). A suitable packaging signal is the HSV-I “a” segment located at approximately nucleotides 127-1132 of the a sequence of the HSV-I virus or its equivalent (Davison et al., J. Gen. Virol. 55:315-331, 1981).

The HSV origin of replication can be any origin of replication that allows for replication of the amplicon vector in the host cell that is to be used for replication and packaging of the vector into HSV amplicon particles. A suitable origin of replication is the HSV-I “c” region, which contains the HSV-I ori segment located at approximately nucleotides 47-1066 of the HSV-I virus or its equivalent (McGeogh et al., Nucl. Acids Res. 14:1727-1745, 1986). Origin of replication signals from other related viruses (e.g., HSV-2 and other herpes viruses, including those listed above) can also be used.

The amplicon plasmids can be prepared (in accordance with the requirements set out herein) by methods known in the art of molecular biology. Empty amplicon vectors can be modified by introducing, at an appropriate restriction site within the vector, a complete transgene (including coding and regulatory sequences). Alternatively, one can assemble only a coding sequence and ligate that sequence into an empty amplicon vector or one that already contains appropriate regulatory sequences (promoter, enhancer, polyadenylation signal, transcription terminator, etc.) positioned on either side of the coding sequence. Alternatively, when using the pHSVlac vector, the LacZ sequence can be excised using appropriate restriction enzymes and replaced with a coding sequence for the transgene. Conditions appropriate for restriction enzyme digests and DNA ligase reactions are well known in the art (see, e.g., Sambrook et al., Molecular Cloning: A Laboratory Manual, Cold Spring Laboratory, Cold Spring Harbor, N.Y. (1989); Ausubel et al. (Eds.), Current Protocols in—Molecular Biology, John Wiley & Sons, New York, N.Y., 1999 and preceding editions; and U.S. Pat. No. 4,237,224).

The amplicon systems featured in these methods and others described herein can all be modified so that the transgene carried by the amplicon plasmid is inserted into the genome of the host cell. Accordingly, the methods described herein can each include an additional step of introducing, into the host cell, a vector (which can be, but is not necessarily, a plasmid) that encodes an enzyme that mediates insertion of the transgene into the genome (this vector may be referred to herein as “an integration vector”). The integration vector can be applied to a host cell in vivo or in culture at the same time that one or more of the components of the amplicon system (e.g. the packaging vector or amplicon plasmid) are administered to the host cell. The enzyme encoded by the integration vector can be a transposase, such as that encoded by sleeping beauty or a biologically active fragment or mutant thereof (i.e., a fragment or mutant of the sleeping beauty sequence that facilitates integration of the transgene into the genome at a rate or to an extent that is comparable to that achieved when wild type sleeping beauty is used). As this system represents a fundamental advance over those in which the amplicon plasmid is maintained outside the genome (and is therefore “diluted out” as cells divide), it has broad application. Methods in which an integration vector is used in the context of an amplicon system, particularly including the hf-HSV systems described herein, can be carried out to treat patients with a wide variety of diseases or disorders (here, as in the methods described above, a “patient” is not limited to a human patient but can be any other type of mammal). For example, the patient can have cancer, an infectious disease, a neurological disease, or be suffering from a neuronal deficit that leads to sensory impairment, such as loss of hearing. Any of the specific types of cancer, infectious diseases, or neurological diseases set out herein can be treated. In addition, one can further modify the amplicon system to improve the safety of treatments in which an integration vector is administered. Frequent transposition events may lead to mutagenesis of the host genome and, possibly, even to proto-oncogene activation (although there is no evidence that this will occur or is likely to occur; we are speculating that the amplicon might enhance the frequency of such events, as 10-15 copies of the transgenon are present within a single virion). To regulate the transposase component of the system more tightly, one could, for example, incorporate the Sleeping Beauty protein into the virion in the form of a fusion with an HSV tegument protein. Alternatively, one could effect exogenous application of transposase protein with the transgenon-containing amplicon vector. Both approaches would prevent continued synthesis of Sleeping Beauty and thus, obviate additional catalysis of transposition. In yet another strategy, one could incorporate protein instability sequences into the open reading frame to limit transposase half-life. As illustrated in the studies below (see Example 15), the transposon in the integration vector should be compatible with sequences flanking the transgene in the amplicon plasmid. For example, where the transposon is of the Sleeping Beauty system, the amplicon vector can include a transgene (for integration) flanked by the Sleeping Beauty terminal repeats. Integrating forms of the HSV amplicon vector platform have been described previously. One form consists of an HSV amplicon backbone and adeno-associated virus (AAV) sequences required for integration [Costantini, 1999 #9726].

The amplicon vector used in any of the methods described herein can also include a sequence that encodes a selectable marker and/or a sequence that encodes an antibiotic resistance gene. Selectable marker genes are known in the art and include, without limitation, galactokinase, beta-galactosidase, chloramphenicol acetyltransferase, beta lactamase, green fluorescent protein (GFP), alkaline phosphate, etc. Antibiotic resistance genes are also known in the art and include, without limitation, ampicillin, streptomycin, spectromycin, etc. A number of suitable empty amplicon vectors have previously been described in the art including, without limitation, pHSVIac (ATCC Accession 40544; U.S. Pat. No. 5,501,979; Stavropoulos and Strathdee, J. Virol., 72:7137-43, 1998), and pHENK (U.S. Pat. No. 6,040,172). The pHSVIac vector includes the HSV-1 a segment, the HSV-1c region, an ampicillin resistance marker, and an E. coli lacZ marker. The pHENK vector includes the HSV-1 a segment, an HSV-1 ori segment, an ampicillin resistance marker, and an E. coli LacZ marker under control of the promoter region isolated from the rat preproenkephalin gene (i.e., a promoter operable in brain cells). The sequences encoding a selectable marker, the sequences encoding the antibiotic resistance gene (which may also serve as a selectable marker), and the sequences encoding the transgene, may be under the control of regulatory sequences such as promoter elements that direct the initiation of transcription by RNA polymerase, enhancer elements, and suitable transcription terminators or polyadenylation signals. Preferably, the promoter elements are operable in the cells of the patient that are targeted for transformation. A number of promoters have been identified that are capable of regulating expression within a broad range of cell types. These include, without limitation, HSV immediate-early 4/5 (IE4/5) promoter, cytomegalovirus (“CMV”) promoter, SV40 promoter, and P-actin promoter. Likewise, a number of other promoters have been identified that can regulate expression within a narrow range of cell types. These include, without limitation, the neural-specific enolase (NSE) promoter, the tyrosine hydroxylase (TH) promoter, the GFAP promoter, the preproenkephalin (PPE) promoter, the myosin heavy chain (MHQ promoter), the insulin promoter, the cholineacetyltransferase (ChAT) promoter, the dopamine β-hydroxylase (DBH) promoter, the calmodulin dependent kinase (CamK) promoter, the c-fos promoter, the c-jun promoter, the vascular endothelial growth factor (VEGF) promoter, the erythropoietin (EPO) promoter, and the EGR-I promoter. The transcription termination signal should, likewise, be operable in the cells of the patient that are targeted for transformation. Suitable transcription termination signals include, without limitation, polyA signals of HSV genes such as the vhs polyadenylation signal, SV40 poly-A signal, and CW IE1 polyA signal.

Conditions Amenable to Treatment

The compositions of the present invention (including herpes virus particles and cells that contain them) can be used to treat: (1) patients who have been, or who may become, infected with a wide variety of agents (including viruses such as a human immunodeficiency virus, human papilloma virus, herpes simplex virus, influenza virus, pox viruses, bacteria, such as E. coli or a Staphylococcus, a parasite, or an unconventional infectious agent such as a prion protein), (2) patients with a wide variety of cancers; (3) patients with a neurological disease or disorder; and (4) patients who have or who may experience hearing loss. A patient can be treated after they have been diagnosed as having a cancer, an infectious disease, or a neurological disorder or, since the agents of the present invention can be formulated as vaccines, patients can be treated before they have developed the cancer, infectious disease or neurological disorder. Thus, “treatment” encompasses prophylactic treatment. Similarly, patients who have experienced a loss of hearing can be treated at any time, including before the loss occurs (e.g., hf-HSV amplicon particles can be administered before the patient is exposed to some agent, such as a chemotherapeutic agent or industrial hazard, that may damage one or more of their senses).

With respect to cancer in general and leukemia in particular, we note that chronic lymphocytic leukemia (CLL) is a malignancy of mature appearing small B lymphocytes that closely resemble those in the mantle zone of secondary lymphoid follicles (Caligaris-Cappio and Hamblin, J. Clin. Oncol. 17:399-408, 1999). CLL remains a largely incurable disease of the elderly with an incidence of more than 20 per 100,000 above the age of 70, making it the most common leukemia in the United States and Western Europe. CLL, which arises from an antigen-presenting B cell that has undergone a non-random genetic event (del13q14-23.1, trisomy 12, del 11q22-23 and del6q21-23 (Dohner et al., J. Mol. Med. 77:266-281, 1999) and clonal expansion, exhibits a unique tumor-specific antigen in the form of surface immunoglobulin. CLL cells possess the ability to successfully process and present this tumor antigen, a characteristic that makes the disease an attractive target for immunotherapy (Bogen et al., Eur. J. Immunol. 16:1373-1378, 1986; Bogen et al., Int. Rev. Immunol. 10:337-355, 1993; Kwak et al., N. Engl. J. Med. 327:1209-1215, 1992; and Trojan et al., Nat. Med. 6:667-672, 2000). However, the lack of expression of co-stimulatory molecules on CLL cells renders them inefficient effectors of T cell activation, a prerequisite for generation of anti-tumor immune responses (Hirano et al., Leukemia 10:1168-1176, 1996). This failure to activate T cells has been implicated in the establishment of tumor-specific tolerance (Cardoso et al., Blood 88:41-48, 1996). Reversal of preexisting tolerance can, potentially, be achieved by up-regulating a panel of co-stimulatory molecules (B7.1, B7.2 and ICAM-I) (Grewal and Flavell, Immunol. Rev. 153:85-106, 1996) through the activation of CD40 receptor-mediated signaling and concomitant enhancement of antigen presentation machinery (Khanna et al., J. Immunol. 159:5982-5785, 1997; Lanzavecchia, Nature 393:413-414, 1998; Diehl et al., Nat. Med. 5:774-779, 1999; Sotomayor et al., Nat. Med. 5:780-787, 1999).

Applying the information above in effective gene therapies for CLL has been hampered by the lack of a safe and reliable vector that can be used to transduce primary leukemia cells. In contrast to tumor cell lines, CLL cells are effectively post-mitotic; only a small fraction of the population enters the cell cycle (Andreeff et al., Blood 55:282-293, 1980). Although both retroviral and adenoviral vectors have been employed in different clinical trials for cancer gene therapy, both systems exhibit limitations (Uckert and Walther, Pharmacol. Ther. 63:323-347, 1994; Vile et al., Mol. Biotechnol. 5:139-158, 1996; Collins, Ernst Schering Research Foundation Workshop, 2000; Hitt et al., Adv. Pharmacol. 40:137-206, 1997; Kochanek, Hum. Gene Ther. 10:2451-2459, 1999). For example, the low levels of integrin receptors for adenovirus on CLL cells mandates the use of very high adenovirus titers, preactivation of the CLL cell with IL-4 and/or anti-CD40/CD40L (Cantwell et al., Blood 88:4676-4683, 1996; Huang et al., Gene Ther. 4:1093-1099, 1997), or adenovirus modification with polycations to achieve clinically meaningful levels of transgene expression (Howard et al., Leukemia 13:1608-1616, 1999).

In some of the Examples below, HSV amplicon particles were used to transduce primary human B-cell chronic lymphocytic leukemia (CLL) cells. The vectors were constructed to encode β-galactosidase (by inclusion of the lacZ gene), B7.1 (also known as CD80), or CD40L (also known as CD154), and they were packaged using either a standard helper virus (HSVlac, HSVB7.1, and HSVCD40L) or by a helper virus-free method (hf-HSVlac, hf-HSVB7.1, and hf-HSVCD40L). CLL cells transduced with these vectors were studied for their ability to stimulate allogeneic T cell proliferation in a mixed lymphocyte tumor reaction (MLTR). A vigorous T cell proliferative response was obtained using cells transduced with hf-HSVB7.1 but not with HSVB7.1. CLL cells transduced with either HSVCD40L or hf-HSVCD40L were also compared for their ability to up-regulate resident B7.1 and function as T cell stimulators. Significantly enhanced B7.1 expression was seen in response to CD40L delivered by hf-HSVCD40L amplicon stock (compared to HSVCD40L). CLL cells transduced with hf-HSVCD40L were also more effective at stimulating T cell proliferation than those transduced with HSVCD40L stocks. These studies support the conclusion that HSV amplicons are efficient vectors for gene therapy, particularly of hematologic malignancies, and that helper virus-free amplicon preparations are better suited for use in therapeutic compositions.

Neuronal diseases or disorders that can be treated include lysosomal storage diseases (treatment can occur, for example, by expressing MPS I-VIII, hexoaminidase A/B, etc.), Lesch Nyhan syndrome (treatment can occur, for example, by expressing HPRT), amyloid polyneuropathy (treatment can occur, for example, by expressing B-amyloid converting enzyme (BACE) or amyloid antisense sequences), Alzheimer's Disease (treatment can occur, for example, by expressing a nerve growth factor such as NGF, ChAT, BACE, etc.), retinoblastoma (treatment can occur by, for example, expressing pRB), Duchenne's muscular dystrophy (treatment can occur by expressing Dystrophin), Parkinson's Disease (treatment can occur, for example, by expressing GDNF, BcI-2, TH, AADC, VMAT, sequences antisense to mutant alpha-synuclein, etc.), Diffuse Lewy Body disease (treatment can occur, for example, by expressing a heat shock protein, parkin, or antisense or siRNA molecules to alpha-synuclein), stroke (treatment can occur by, for example, expressing Bc1-2, HIF-DN, BMP7, GDNF, or other growth factors), brain tumor (treatment can occur by, for example, expressing angiostatin, antisense VEGF, antisense or ribozyme to EGF or scatter factor, or pro-apoptotic proteins), epilepsy (treatment can occur by, for example, expressing GAD65, GAD67, or pro10 apoptotic proteins into focus), or arteriovascular malformation (treatment can occur by expressing proapoptotic proteins).

Therapeutic Agents

As noted, the hf-HSV amplicon particles described herein (and the cells that contain them) can express a heterologous protein (i.e., a full-length protein or a portion thereof (e.g., a functional domain or antigenic peptide) that is not naturally encoded by a herpes virus). The heterologous protein can be any protein that conveys a therapeutic benefit on the cells in which it, by way of infection with an hf-HSV amplicon particle, is expressed or a patient who is treated with those cells.

The therapeutic agents can be immunomodulatory (e.g., immunostimulatory) proteins (as described in U.S. Pat. No. 6,051,428). For example, the heterologous protein can be an interleukin (e.g., IL-1, IL-2, IL-4, IL-10, or IL-15), an interferon (e.g., IFNγ), a granulocyte macrophage colony stimulating factor (GM-CSF), a tumor necrosis factor (e.g., TNFα), a chemokine (e.g., RANTES, MCP-1, MCP-2, MCP-3, DC-CK1, MIP-1α, MIP-3α, MIP-β, MIP-3β, an α or C—X—C chemokine (e.g., IL-8, SDF-1β, 8DF-1α, GRO, PF-4 and MIP-2). Other chemokines that can be usefully expressed are in the C family of chemokines (e.g., lymphotactin and CX3C family chemokines).

Intercellular adhesion molecules are transmembrane proteins within the immunoglobulin superfamily that act as mediators of adhesion of leukocytes to vascular endothelium and to one another. The vectors described herein can be made to express ICAM-1 (also known as CD54), and/or another cell adhesion molecule that binds to T or B cells (e.g., ICAM-2 and ICAM-3).

Costimulatory factors that can be expressed by the vectors described herein are cell surface molecules, other than an antigen receptor and its ligand, that are required for an efficient lymphocytic response to an antigen (e.g., B7 (also known as CD80) and CD40L).

When used for gene therapy, the transgene encodes a therapeutic transgene product, which can be either a protein or an RNA molecule.

Therapeutic RNA molecules include, without limitation, antisense RNA, inhibitory RNA (siRNA), and an RNA ribozyme. The RNA ribozyme can be either cis or trans acting, either modifying the RNA transcript of the transgene to afford a functional RNA molecule or modifying another nucleic acid molecule. Exemplary RNA molecules include, without limitation, antisense RNA, ribozymes, or siRNA to nucleic acids for huntingtin, alpha synuclein, scatter factor, amyloid precursor protein, p53, VEGF, etc.

Therapeutic proteins include, without limitation, receptors, signaling molecules, transcription factors, growth factors, apoptosis inhibitors, apoptosis promoters, DNA replication factors, enzymes, structural proteins, neural proteins, and histone or non-histone proteins. Exemplary protein receptors include, without limitation, all steroid/thyroid family members, nerve growth factor (NGF), brain derived neurotrophic factor (BDNF), neutotrophins 3 and 4/5, glial derived neurotrophic factor (GDNF), cilary neurotrophic factor (CNTF), persephin, artemin, neurturin, bone morphogenetic factors (BM1's), c-ret, gp 130, dopamine receptors (D 1D5), muscarinic and nicotinic cholinergic receptors, epidermal growth factor (EGF), insulin and insulin-like growth factors, leptin, resistin, and orexin. Exemplary protein signaling molecules include, without limitation, all of the above-listed receptors plus MAPKs, ras, rac, ERKs, NFKβ, GSK3β, AKT, and PI3K. Exemplary protein transcription factors include, without limitation, ˜300, CBP, HIF-1alpha, NPAS1 and 2, HIF-1β, p53, p73, nurr 1, nurr 77, MASHs, REST, and NCORs. Exemplary neural proteins include, without limitation, neurofilaments, GAP-43, SCG-10, etc. Exemplary enzymes include, without limitation, TH, DBH, aromatic amino acid decarboxylase, parkin, unbiquitin E3 ligases, ubiquitin conjugating enzymes, cholineacetyltransferase, neuropeptide processing enzymes, dopamine, VMAT and other catecholamine transporters. Exemplary histones include, without limitation, H1-5. Exemplary non-histones include, without limitation, ND10 proteins, PML, and HMG proteins. Exemplary pro-and anti-apoptotic proteins include, without limitation, bax, bid, bak, bc1-xs, bc1-x1, bc1-2, caspases, SMACs, and IAPs.

Formulation and Administration of hf-HSV Amplicon Particles

The hf-HSV amplicon particles described herein can be administered to patients directly or indirectly; alone or in combination with other therapeutic agents; and by any route of administration. For example, the hf-HSV amplicon particles can be administered to a patient indirectly by administering cells transduced with the vector to the patient. Alternatively, or in addition, an hf-HSV amplicon particle could be administered directly. For example, an hf-HSV amplicon particle that expresses an immunostimulatory protein or a tumor-specific antigen can be introduced into a tumor by, for example, injecting the vector into the tumor or into the vicinity of the tumor (or, in the event the cancer is a blood-bourne tumor, into the bloodstream).

Administration of HSV-immunomodulatory protein amplicons encoding cytokines such as IL-2, GM-CSF and RANTES, intercellular adhesion molecules such as ICAM-1 and costimulatory factors such as B7.1 all provide therapeutic benefit in the form of reduction of preexisting tumor size, a vaccine-effect protecting against tumor growth after a subsequent challenge, or both (see U.S. Pat. No. 6,051,428; see also Kutubuddin et al., Blood 93:643-654, 1999). The helper virus-free HSV vectors disclosed herein can be administered in the same manner.

The herpes virus amplicon particles described herein, and cells that contain them, can be administered, directly or indirectly, with other species of HSV-transduced cells (e.g., HSV-immunomodulatory transduced cells) or in combination with other therapies, such as cytokine therapy. Such administrations may be concurrent or they may be done sequentially. Thus, in one embodiment, HSV amplicon particles, the vectors with which they are made (i.e., packaging vectors, amplicon plasmids, and vectors that express an accessory protein) can be injected into a living organism or patient (e.g. a human patient) to treat, for example, cancer or an infectious disease. In further embodiments, one or more of these entities can be administered after administration of a therapeutically effective amount of a cytokine.

The concentrated stock of HSV amplicon particles is effectively a composition of the HSV amplicon particles in a suitable carrier. HSV amplicon particles can also be administered in injectable dosages by dissolving, suspending, or emulsifying them in physiologically acceptable diluents with a pharmaceutical carrier (at, for example, about 1×10⁷ amplicon particles per ml). Such carriers include sterile liquids, such as water and oils, with or without the addition of a surfactant and other pharmaceutically and physiologically acceptable carriers, including adjuvants, excipients or stabilizers. The oils that can be used include those obtained from animals or vegetables, petroleum based oils and synthetic oils. For example, the oil can be a peanut, soybean, or mineral oil. In general, water, saline, aqueous dextrose and related sugar solutions, glycols (e.g., propylene glycol or polyethylene glycol) are preferred liquid carriers, particular when the amplicon particles are formulated for administration by injection.

For use as aerosols, the HSV amplicon particles, in solution or suspension, can be packaged in a pressurized aerosol container together with suitable propellants, for example, hydrocarbon propellants like propane, butane, or isobutene with conventional adjuvants. The particles can also be administered in a non-pressurized form such as in a nebulizer or atomizer.

### EXAMPLES

### Example 1

### HSV Amplicon Vector-Mediated Transduction of Murine Dendritic Cells

We have constructed amplicon particles that encode the model tumor antigen ovalbumin (HSV-OVA) and human prostate-specific antigen (HSV-PSA), a protein that is expressed specifically in prostate epithelium and prostate carcinoma cells.

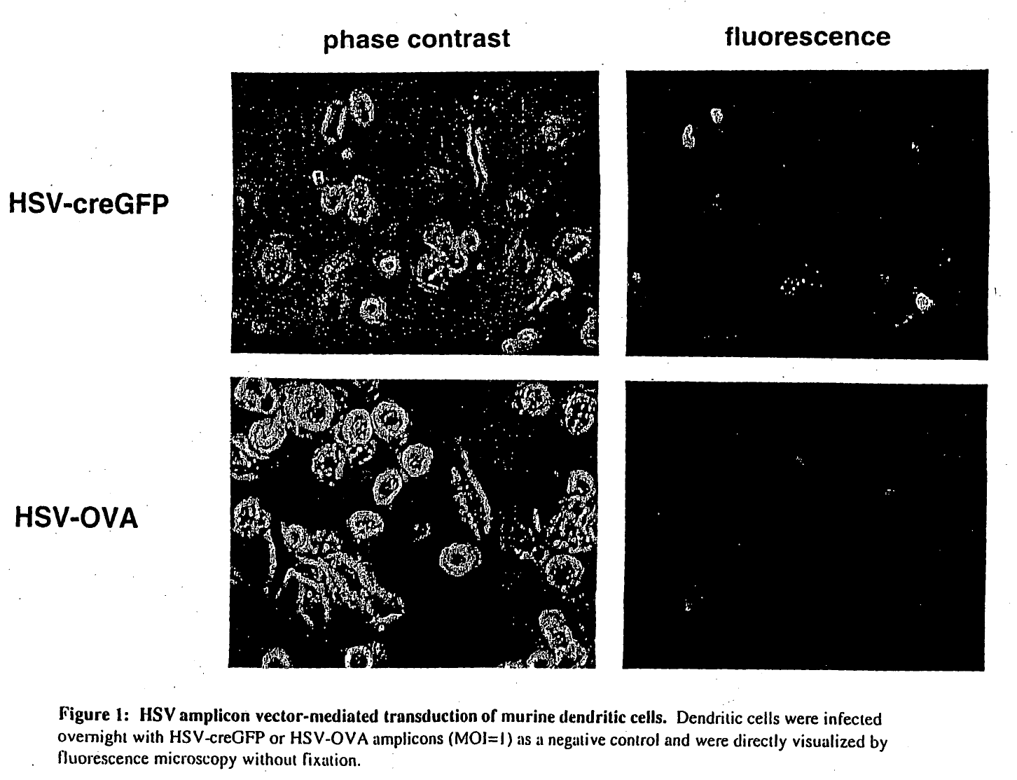

As shown in FIG. 1, dendritic cells can be transduced with HSV amplicons. Murine dendritic cells were infected overnight with HSV-creGFP or, as a negative control, a comparable vector that did not include a fluorescent marker (HSV-OVA). The cells were viewed under a microscope (without fixation) with phase contrast optics and with fluorescent light appropriate for visualizing GFP. The cells, as they appeared by phase contrast following transduction with the HSV-creGFP amplicon and the HSV-OVA amplicion, are shown in the upper and lower left-hand panels of FIG. 1, respectively. When viewed with fluorescent light, the cells successfully transduced with the HSV-creGFP amplicon fluoresce (upper right-hand panel of FIG. 1), but none of the HSV-OVA-transduced cells do (lower right-hand panel of FIG. 1).

### Example 2

### Dendritic Cells Transduced with HSV Amplicons Present Antigen to T Cell Hybridomas

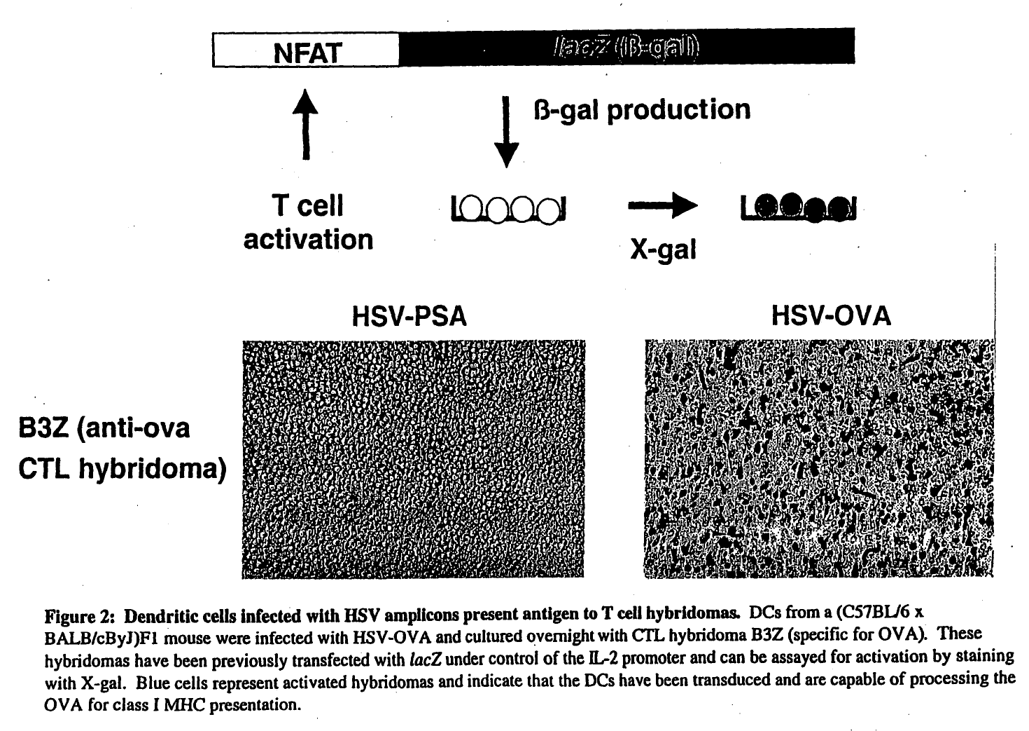

As in Example 1, murine dendritic cells (obtained from a C57B1/6× BALB/cByJ)F1 mouse) were infected with an HSV-OVA amplicon and, as a negative control, a comparable population of dendritic cells were infected with an HSV-PSA amplicon. The dendritic cells were then cultured overnight with CTL hybridoma B3Z cells that (1) have been transfected with a construct in which the lacZ gene, encoding β-galactosidase, is placed under the control of an IL-2 promoter (NFAT) and (2) become activated in the presence of ovalbumin. (We have also developed class I-restricted CTL hybridomas specific for PSA). The construct is illustrated at the top of FIG. 2. Following T cell activation, the NFAT promoter is bound, the lacZ gene is transcribed, and the cells in which β-galactosidase is produced turn blue upon staining with X-gal (a standard assay). The hybridoma cells, as they appear following X-gal staining, are shown in the lower half of FIG. 2. No T cells co-cultured with HSV-PSA-transfected dendritic cells turned blue (left-hand photograph), but many of those co-cultured with HSV-OVA-transfected cells did (right-hand panel). The fact that T cells were activated means that the dendritic cells were not only successfully transduced, but also processed OVA for class I MHC presentation.

Infection of DCs with HSV-PSA and co-culture with CTL hybridomas specific for PSA can be used to evaluate presentation of PSA. In fact, infection with an HSV-based amplicon that expresses any antigen of interest can be similarly tested for presentation.

### Example 3

### Mice Immunized with HSV Amplicon-Transduced Dendritic Cells Respond by Producing Antigen-Specific Cytotoxic T Lymphocytes

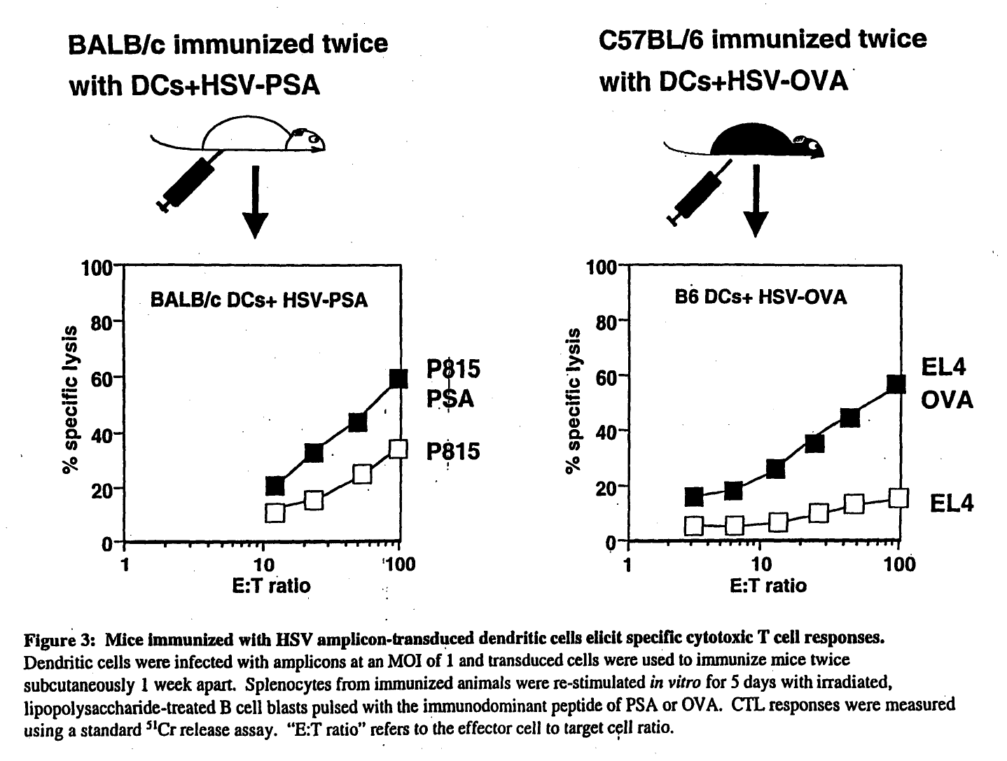

Dendritic cells were infected in cell culture with one of two amplicons: an HSV-PSA amplicon or an HSV-OVA amplicon, each at an MOI of 1. The transduced cells were used to immunize mice (BALB/c mice were immunized with HSV-PSA-transduced dendritic cells and C57B1/6 mice were immunized with HSV-OVA-transduced dendritic cells, as illustrated in FIG. 3). The cells were injected subcutaneously on day 1 and day 7. Splenocytes were subsequently obtained from the immunized animals and placed in cell culture where they were re-stimulated for five days with irradiated, lipopolysaccharide-treated B cells blasts with the immunodominant peptide of PSA or OVA. CTL responses were measured using a standard ⁵¹Cr release assay. The results, which are presented in FIG. 3 as plots of % specific lysis vs. E:T ratio (the ratio of effector cell to target cell), demonstrate that mice immunized with dendritic cells infected with HSV-OVA or HSV-PSA generate specific CTL responses that can be detected in vitro.

### Example 4

### Dendritic Cells Infected with HSV-p35 Amplicons and Activated with CpG Oligonucleotides Produce Increased Levels of IL-12 p70 Heterodimer

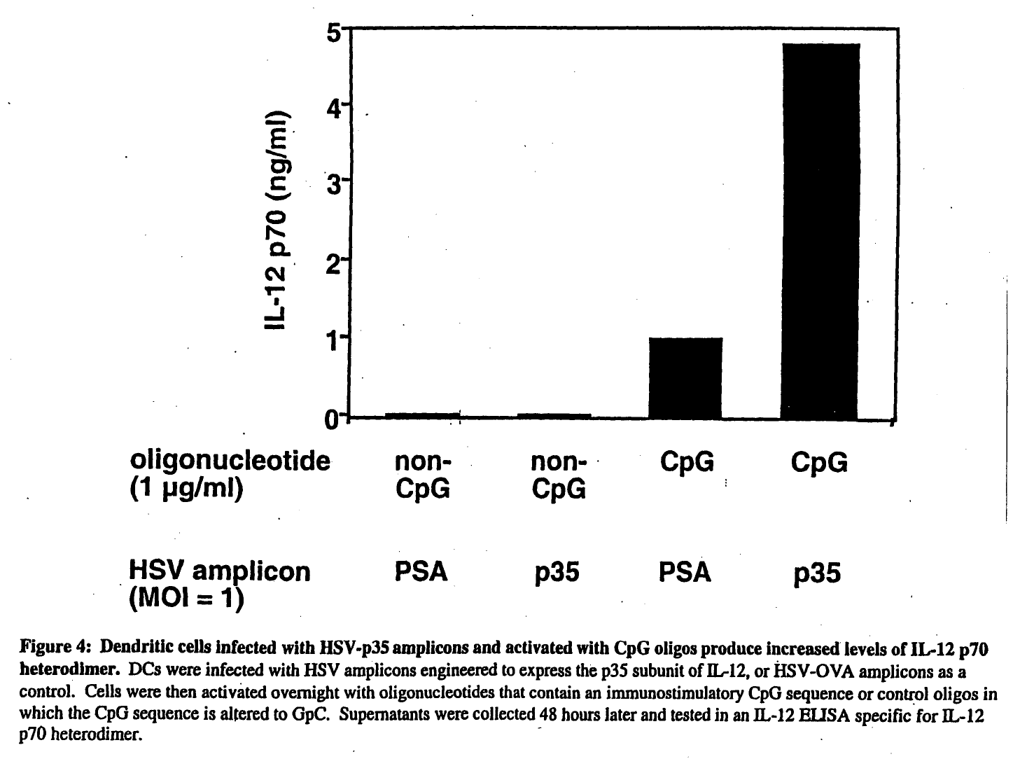

We have also used amplicons to express IL-12 in activated DCs to enhance Th1-mediated responses (FIG. 4). IL-12 is a product of activated APCs and is an important activator of NK and T cell responses. Dendritic cells were infected in cell culture with one of two amplicons: an HSV-PSA amplicon (which served as a control) or an HSV-p35 amplicon (p35 is a subunit of IL-12). Following infection, the dendritic cells were activated with oligonucleotides that contain an immunostimulatory sequence (CpG) or with control oligonucleotides in which the CpG sequence is altered to GpC. Supernatants were collected 48 hours later and tested in an IL-12 ELISA specific for IL-12 p70 heterodimer. As shown in FIG. 4, IL-12 p70 expression was almost nil in cells that were infected with either HSV-PSA or HSV-p35 and stimulated with the control oligonucleotides. There was a low level of IL-12 p70 expression when HSV-PSA-infected cells were stimulated with CpG oligonucleotides and robust expression from HSV-p35-infected cells stimulated with CpG oligonucleotides. These experiments demonstrate that, as shown above, dendritic cells can be successfully transduced with HSV-based amplicons and that the antigen encoded by the amplicon can be induced by appropriate stimuli.

Taken together, the studies described above support the use of DCs infected with HSV-1 amplicon particles in investigations of CTL activation and in immunotherapies to treat cancer and other diseases. The studies described herein provide direct evidence that these HSV-based amplicons can effectively infect cells that remain functional in their ability to present antigen, which is crucial to their use as therapeutic agents (e.g., when formulated as vaccines).

### Example 5

### Fibroblasts Infected with an HSV-gp120 Amplicon Express gp120

Immunotherapeutic agents for the treatment of HIV infection are likely to be more effective if they can induce or enhance CD4⁺- and CD8⁺-T cell activity. To develop such agents, we generated an amplicon vector that encodes the HIV envelope glycoprotein (HSVgp120). The construct was packaged using a modified BAC-based expression system, and gp120 expression was initially monitored by Western blot analysis. As described further below, NIH 3T3 cells infected with HSVgp120 produced high levels of the HIV glycoprotein.

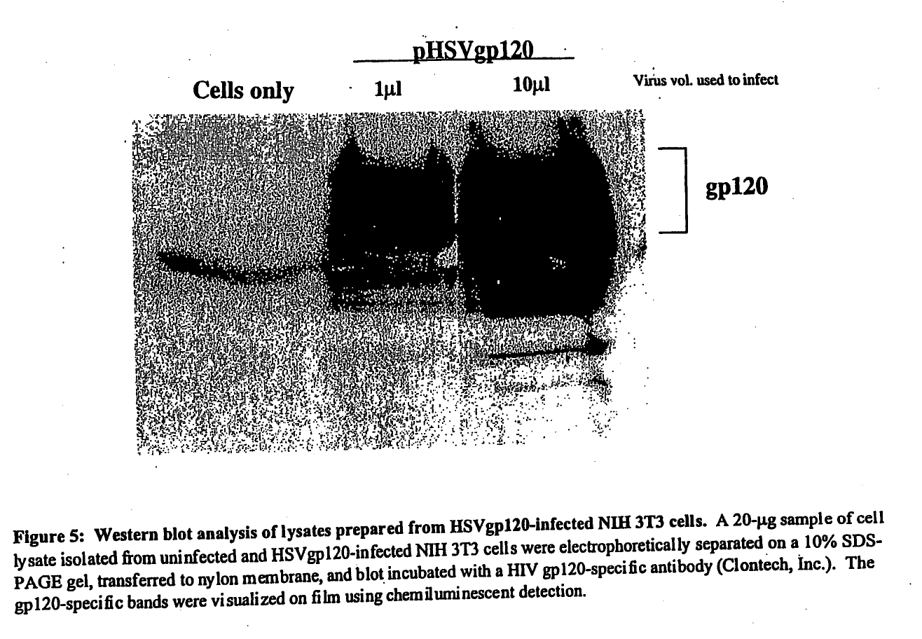

NIH 3T3 cells were cultured and infected with an HSV-gp120 amplicon. Lysates were then prepared and the proteins in them were analyzed. More specifically, 20 μg samples of cell lysates were isolated from uninfected NIH 3T3 cells (this sample served as a control) and HSV-gp120-infected NIH 3T3 cells, separated electrophoretically on a 10% SDS-polyacrylamide gel, and transferred to a nylon membrane that was incubated with an HIV gp120-specific antibody (Clontech, Inc.). The gp120-specific bands were visualized on film using chemiluminescent detection. As shown in FIG. 5, uninfected cells expressed virtually no gp120, whereas HSV-gp120-infected cells expressed substantial amounts of this protein. The lanes designated 1 μl and 10 μl in FIG. 5 represent two different volumes of virus stock used to infect the cells. This high level of expression demonstrates that fibroblasts can be readily infected with an HSV amplicon.

### Example 6

### Animals Immunized with an HSV-gp120 Amplicon Display a Cell-Mediated Immune Response

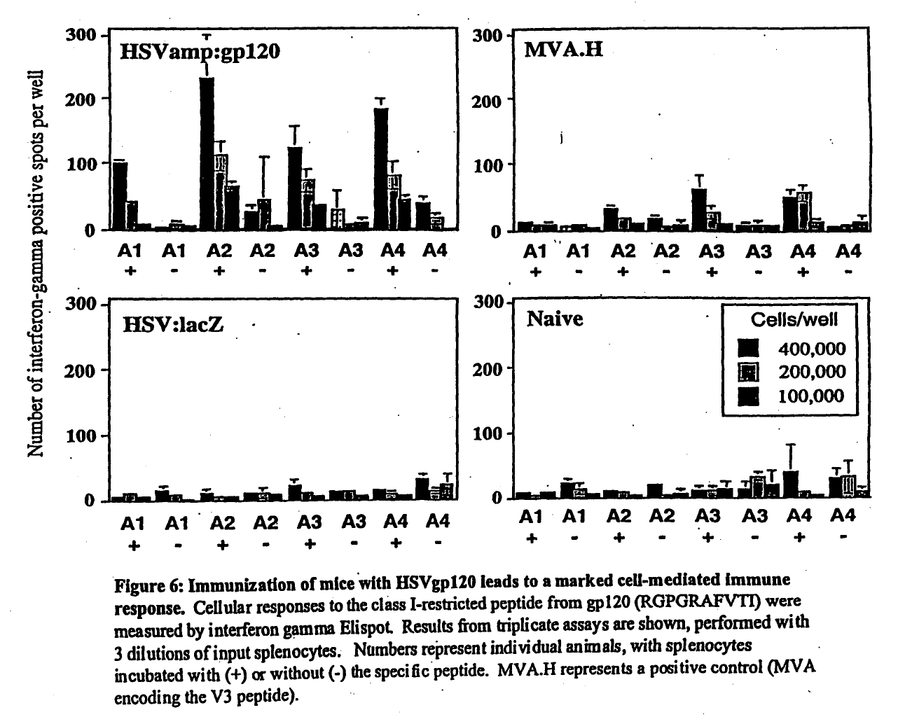

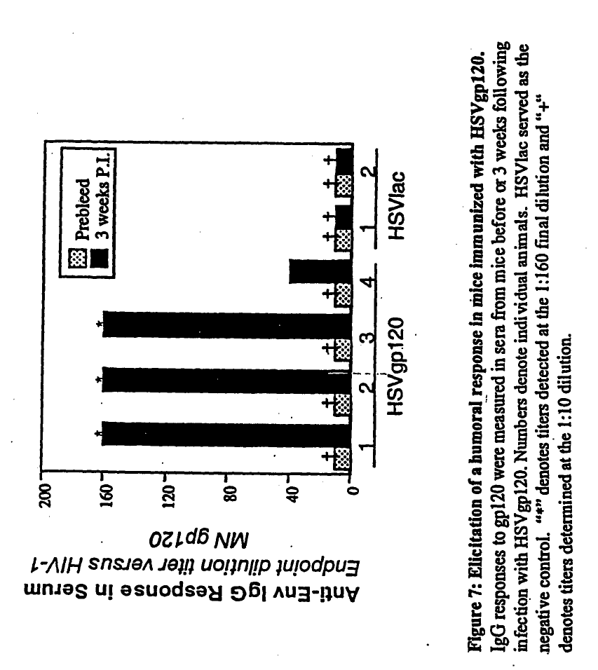

We next tested the ability of the HSV-gp120 vector to elicit gp120-specific immune responses in BALB/c mice. We were able to detect strong responses to a single intramuscular injection, at both the humoral and cellular level. Anti-Env IgG antibodies were generated (see below and FIG. 6). Cellular immune responses were detected in an interferon-gamma Elispot assay using the class I-restricted V3 peptide recognized by the mice (RGPGRAFVT (SEQ ID NO:1); see Example 7 and FIG. 7)). In these experiments, HSV amplicons expressing a modified MN gp120 induced interferon gamma-producing T cells that were equivalent to those induced by live herpes virus vectors, and that far exceeded those induced by a modified vaccinia Ankara vector.

To determine whether animals immunized with an HSV-gp120 amplicon could later mount a cell-mediated immune response to the gp120 antigen, mice were immunized with either (1) an HSV-gp120 amplicon, (2) a sequence encoding the V3 peptide (MVA.H), or (3) an HSV-lacZ amplicon. “Naïve” mice constituted a fourth group. Following immunization, the mice were sacrificed and their splenocytes were placed in culture. The cellular responses to a class I-restricted peptide from gp120 (RGPGRAFVTI (SEQ ID NO:1)) were measured by interferon gamma Elispot. Splenocytes incubated without the gp120 peptide served as another control for this study. The number of interferon-gamma-positive spots per well was plotted for each animal, in triplicate, with three dilutions of input splenocytes (100,000; 200,000; and 400,000 cells/well). The results are shown in FIG. 6. The designations A1-A4 represent splenocytes obtained from individual animals, and the (+) and (−) symbols beneath those designations mark splenocytes incubated with or without the specific gp120 peptide. As shown in FIG. 6, the number of interferon gamma-positive spots (which is indicative of the ability of the cells to mount a cell-mediated immune response) was low and not significantly different in splenocytes obtained from mice that were immunized with MVA or HSV-lacZ or that were not immunized at all (naïve). However, significantly more of the splenocytes obtained from HSV-gp120-immunized mice produced interferon following exposure to the gp120 peptide in culture.

### Example 7

### Animals Infected with HSV-gp120 also Exhibit a Humoral Immune Response

Mice were immunized with either an HSV-gp120 amplicon or an HSV-lacZ amplicon (which served as a negative control). Serum was obtained either before the animals were infected or three weeks afterward and analyzed for anti-env IgG antibodies. The results are shown in FIG. 7. The numbers on the y-axis represent individual animals (four were immunized with HSV-gp120 and two were immunized with HSV-lacZ); the astericks above some bars of the graph represent titers detected at the 1:160 final dilution; and the “+” above other bars denotes titers determined at the 1:10 dilution. The anti-env IgG response in serum obtained three weeks after immunization with HSV-gp120 was substantially greater than in serum obtained from the animals prior to immunization or in serum obtained from animals immunized with HSV-lacZ. Thus, humoral as well as cell-mediated immune responses result.

### Example 8

### HSV-gp120 Induces CTL Activity In Vivo

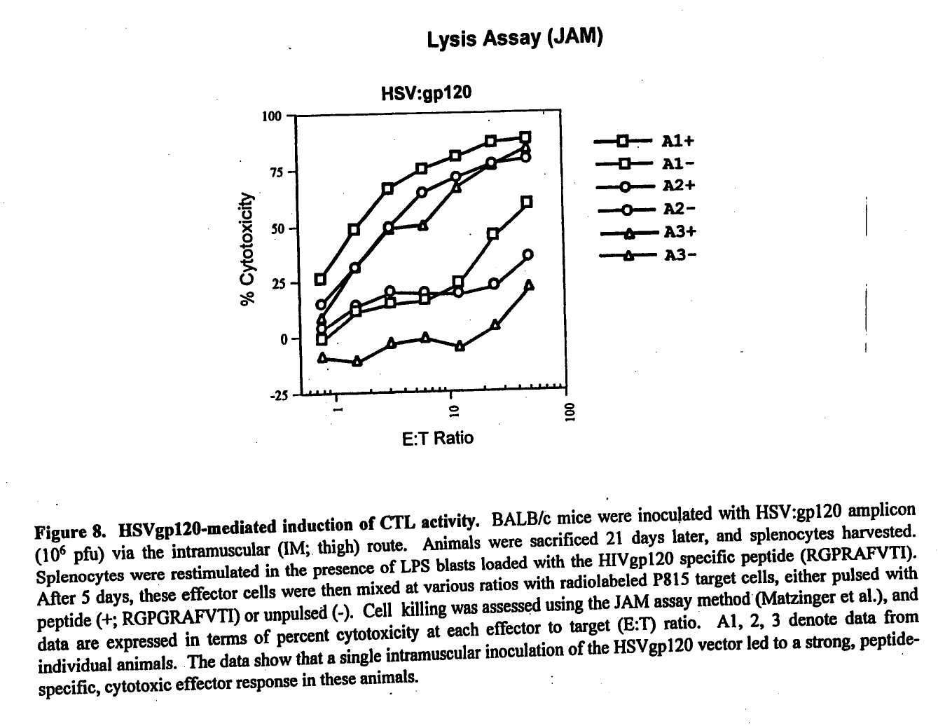

BALB/c mice (n=3) were inoculated with an HSV-gp120 amplicon (10⁶ pfu) by intramuscular injection. The mice were sacrificed 21 days later, and splenocytes were harvested and placed in culture, where they were restimulated in the presence of LPS blasts loaded with the HIVgp120 specific peptide RGPRAFVTI (SEQ ID NO:1). After five days, these effector cells were mixed at various ratios with radiolabeled P815 target cells, either pulsed with peptide (+) or unpulsed (−). Cell killing was assessed using the JAM assay method described by Matzinger et al. (J. Immunol. Methods 145:185-92, 1991). The data, shown in FIG. 8, were expressed in terms of % cytotoxicity at each effector to target (E:T) ratio. A1, A2, and A3 denote data obtained from individual animals. These data demonstrate that a single intramuscular injection of an HSV-gp120 vector is sufficient to produce a strong, peptide-specific, cytotoxic effector response in the treated animals.

### Example 9

### Subcutaneous Administration of an HSV-gp120 Amplicon can Produce a Greater Cellular Immune Response than Other Routes of Administration

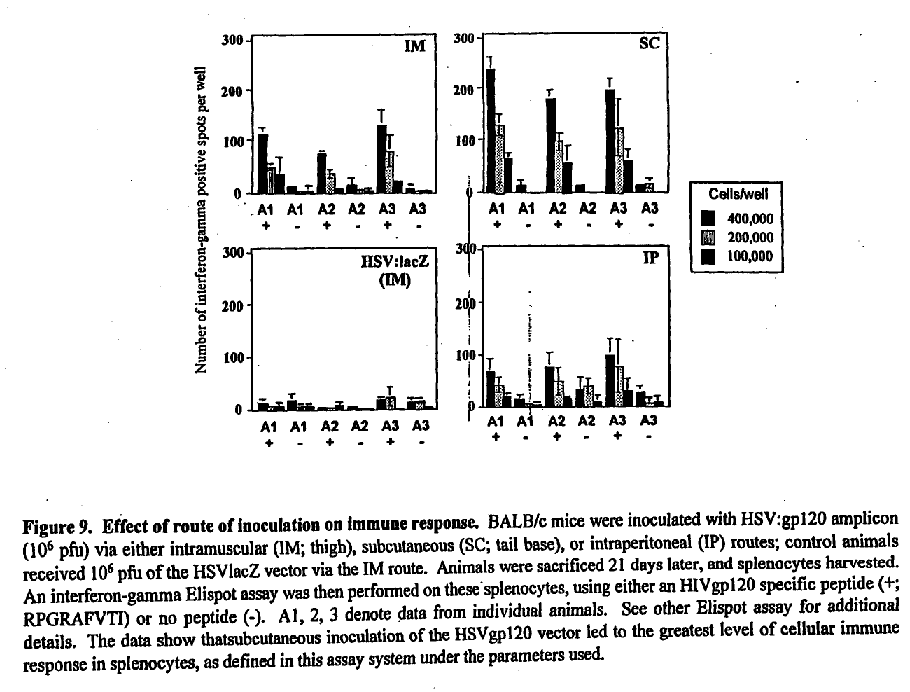

To study the effect of the route of administration on the strength of the immune response generated, BALB/c mice were inoculated with the same vector, an HSV-gp120 amplicon (10⁶ pfu) administered either intramuscularly (into the thigh), subcutaneously (at the base of the tail), or intraperiotoneally. Control mice received 10⁶ pfu of the HSV-lacZ vector intramuscularly. All animals were sacrificed 21 days later, and their splenocytes were harvested and subjected to an interferon-gamma Elispot assay using either an HIVgp120 specific peptide (RGPRAFVTI (SEQ ID NO:1); designated “+” in FIG. 9) or no peptide (designated “−” in FIG. 9). A1, A2, and A3 designate splenocytes obtained from individual animals. As shown in FIG. 9, while all routes of administration produced some number of interferon-gamma-positive spots per well, the greatest number were produced when the antigen had been administered subcutaneously. Thus, subcutaneous inoculation with HSV-gp120 produced the best cellular immune response (at least as defined in this assay system under the parameters used).

The experiments described above show that amplicons can infect DCs, which function in vitro and in vivo. Moreover, direct injection of amplicons results in effective immunization in vivo. Thus, these vectors provide a useful platform for a variety of antigens, including HIV antigens, and the HSV amplicon-based vector systems described herein can be used to treat HIV infection.

### Example 10

### Production of a Helper Virus-Free Amplicon Particle

As noted above, HSV-based amplicon particles are attractive gene delivery tools, and they are particularly well suited for delivering gene products to neurons (e.g. neurons in the central nervous system) because they are easy to manipulate, can carry large transgenes, and are naturally neurotropic (Geller and Breakefield, Science 241:1667-1669, 1988; Spaete and Frenkel, Cell 30:305-310, 1982; Federoff et al., Proc. Natl. Acad. Sci. USA 89:1636-1640, 1992; Federoff in Cells: A Laboratory Manual, Spector et al., Eds., Cold Spring Harbor Press, Cold Spring Harbor, N.Y., 1997; Frenkel et al., in Eucaryotic Viral Vectors, Gluzman, Ed., Cold Spring Harbor Press, Cold Spring Harbor, N.Y., 1982). Efforts to bring this vector system into the clinical arena to treat neurodegenerative disease have been hampered by potential cytotoxicites that are associated with traditional methods of virus packaging. This problem involves the co-packaging of helper virus that encodes cytotoxic and immunogenic viral proteins. Newer methods of packaging have been developed that result in helper virus-free amplicon stocks (Fraefel et al., J. Virol. 70:7190-7197, 1996; Stavropoulos and Strathdee, J. Virol. 72:7137-7143, 1998; see also U.S. Pat. Nos. 5,851,826 and 5,998,208). Stocks prepared by these methods, however, are typically low titer (<10⁵ expression units/ml), allowing for only modest scale experimentation, primarily in vitro. Such low titers make large animal studies difficult, if not impossible. Present helper virus-free packaging strategies lead to not only lower amplicon titers, but also to stocks that exhibit a high frequency of pseudotransduction events when used to infect a variety of cell types.

Optimal propagation of wild-type HSV virions requires orderly progression of α, β, and γ gene transcription following infection of a host cell. This is achieved by delivery of co-packaged proteins, carried by the virion, that help co-opt the cell's transcription machinery and transactivation of viral a gene promoters. This information is fundamental to the development of our helper virus-free system. Helper virus-based packaging involves superinfection of an amplicon DNA-transfected monolayer of packaging cells with a replication-defective helper virus. The helper virus genome, as in the case of wild-type HSV, is delivered to the cell in a complex with co-packaged proteins, including VP16 and virion host shutoff (vhs). The HSV vhs protein functions to inhibit the expression of genes in infected cells via destabilization of both viral and host mRNAs. Because vhs plays such a vital role in establishing the HSV replicative cycle and is a potential structural protein, we hypothesized that its presence during amplicon packaging accounted for the higher titers obtained with helper virus-based packaging systems. VP16 is another co-packaged protein that resides in the helper virus nucleocapsid and is responsible for activating transcription of HSV immediate-early genes to initiate the cascade of lytic cycle-related viral protein expression.

In contrast to helper virus-based packaging systems, helper virus-free systems involve co-transfection of naked DNA forms of either an HSV genome-encoding cosmid set or BAC reagent with an amplicon vector (e.g., a plasmid). Thus, the HSV genome gains access to the cell without co-packaged vhs or VP16. The initiation and temporal progression of HSV gene expression is, we speculated, not optimal for production of packaged amplicon vectors due to the absence of these important HSV proteins. To test our hypothesis—that the efficiency of amplicon packaging would be increased by introducing vhs and/or VP16 during the initial phase of virus propagation—we included a vhs-encoding DNA segment in the packaging protocol as a co-transfection reagent. In some instances, packaging cells were “pre-loaded” with VP16 to mimic its presence during helper virus-mediated amplicon packaging. As shown below, these modifications led to a 30- to 50-fold enhancement of packaged amplicon vector titers, nearly approximatig titers obtained using helper virus-based traditional approaches. In addition, the viral stocks failed to exhibit the pseudotransduction phenomenon. These improvements make large-scale in vivo applications much more likely. The methods used to make a helper virus-free amplicon particles are described first, followed by a description of the results obtained.

Cell culture: Baby hamster kidney (BHK) cells were maintained as described by Lu et al. (Human Gene Ther. 6:421-430, 1995). NIH 3T3 cells were originally obtained from the American Type Culture Collection and were maintained in Dulbecco's modified Eagle medium (DMED) supplemented with 10% fetal bovine serum, penicillin, and streptomycin.

Plasmid construction: The HSVPrPUC/CMVegfp amplicon plasmid was constructed by cloning the 0.8-kb cytomegalovirus (CMV) immediate early promoter and 0.7-kb enhanced green fluorescent protein cDNA (Clontech, Inc.) into the BamHI restriction enzyme site of the pHSVPrPUC amplicon vector (Geller et al., Proc. Natl. Acad. Sci. USA 87:8950-8954, 1990). A 3.5 kb HpaI/HindIII fragment encompassing the UL41 (vhs) open reading frame and its 5′ and 3′ transcriptional regulatory elements was removed from cos56 (Cunningham and Davison, Virol. 197:116-124, 1993) and cloned into pBSKSII (Stratagene, Inc.) to create pBSKS(vhs). For construction of pGRE₅vp16, the VP16 coding sequence was amplified by PCR from pBAC-V2 using gene-specific oligonucleotides that possess EcoRI (5′-CGGAATTCCGCAGGTTTTGTAATGTATGTGCTCGT-3′ (SEQ ID NO:2) and HindIII (5′-CTCCGAAGCTTAAGCCCGATATCGTCTTTCCCGTATCA-3′ (SEQ ID NO:3)) restriction enzyme sequences that facilitate cloning into the pGRE₅-2 vector (Mader and White, Proc. Natl. Acad. Sci. USA 90:5603-5607, 1993).

Helper virus-free Amplicon Packaging: On the day prior to transfection, 2×10⁶ BHK cells were seeded on a 60-mm culture dish and incubated overnight at 37° C. The following procedures were followed for cosmid-based packaging. The day of transfection, 250 μl Opti-MEM (Gibco-BRL, Bethesda, Md.), 0.4 μg of each of five cosmid DNAs (kindly provided by Dr. A. Geller, and 0.5 μg amplicon vector DNA, with or without varying amounts of pBSKS(vhs) plasmid DNA were combined in a sterile polypropylene tube (Fraefel et al., J. Virol. 70:7190-7197, 1996). The following procedures were followed for BAC-based packaging. 250 μl Opti-MEM (Gibco-BRL, Bethesda, Md.), 3.5 μg of pBAC-V2 DNA (kindly provided by Dr. C. Strathdee, and 0.5 μg amplicon vector DNA, with or without varying amounts of pBSKS(vhs) plasmid DNA were combined in a sterile polypropylene tube (Stavropoulos and Strathdee, J. Virol. 72:7137-7143, 1998). The protocol for both cosmid- and BAC-based packaging was identical from the following step forward. Ten microliters of Lipofectamine Plus™ reagent (Gibco-BRL) were added over a 30-second period to the DNA mix and allowed to incubate at room temperature for 20 minutes. In a separate tube, 15 μL Lipofectamine (Gibco-BRL) were mixed with 250 μl Opti-MEM. Following the 20 minute incubation, the contents of the two tubes were combined over a one-minute period and then incubated for an additional 20 minutes at room temperature. During the second incubation, the medium in the seeded 60 mm dish was removed and replaced with 2 ml Opti-MEM. The transfection mix was added to the flask and allowed to incubate at 37° C. for five hours. The transfection mix was then diluted with an equal volume of DMEM plus 20% FBS, 2% penicillin/streptomycin, and 2 mM hexamethylene bis-acetamide (HMBA), and incubated overnight at 34° C. The following day, medium was removed and replaced with DMEM plus 10% FBS, 1% penicillin/streptomycin, and 2 mM HMBA. The packaging flask was incubated an additional three days and virus was harvested and stored at −80° C. until purification. Viral preparations were subsequently thawed, sonicated, and clarified by centrifugation (3000×g for 20 minutes). Viral samples were stored at −80° C. until use.

For concentrated viral stocks, viral preparations were subsequently thawed, sonicated, clarified by centrifugation, and concentrated by ultracentrifugation through a 30% sucrose cushion (Geschwind et al., Providing pharmacological access to the brain in Methods in Neuroscience, Conn, Ed., Academic Press, Orlando, Fla., 1994). Viral pellets were resuspended in 100 μl PBS and stored at −80° C. until use. For packaging experiments examining the effect of VP16 on amplicon titers, the cells plated for packaging were first allowed to adhere to the 60 mm culture dish for 5 hours and subsequently transfected with pGRE₅vp16 using the Lipofectamine reagent as described above. Following a five-hour incubation, the transfection mix was removed, complete medium (DMEM plus 10% FBS, 1% penicillin/streptomycin) was added, and the cultures were incubated at 37° C. until the packaging co-transfection step the next day.

Viral titering: Amplicon titers were determined by counting the number of cells expressing enhanced green fluorescent protein (HSVPrPUC/CMVegfp amplicon) or β-galactosidase (HSVlac amplicon). Briefly, 10 μl of concentrated amplicon stock was incubated with confluent monolayers (2×10⁵ expressing particles) of NIH 3T3 cells plated on glass coverslips. Following a 48-hr incubation, cells were either fixed with 4% paraformaldehyde for 15 min at RT and mounted in Mowiol for fluorescence microscopy (eGFP visualization), or fixed with 1% glutaraldehyde and processed for X-gal histochemistry to detect the lacZ transgene product. Fluorescent or X-gal-stained cells were enumerated, expression titer calculated, and represented as either green-forming units per ml (gfu/ml) or blue-forming units per ml (bfu/ml), respectively.

TaqMan Quantitative PCR System: To isolate total DNA for quantitation of amplicon genomes in packaged stocks, virions were lysed in 100-mM potassium phosphate pH 7.8 and 0.2% Triton X-100. Two micrograms of genomic carrier DNA was added to each sample. An equal volume of 2× Digestion Buffer (0.2 M NaCl, 20 mM Tris-Cl pH 8.0, 50 mM EDTA, 0.5% SDS, 0.2 mg/ml proteinase K) was added to the lysate and the sample was incubated at 56° C. for 4 hrs. Samples were processed further by one phenol:chloroform, one chloroform extraction, and a final ethanol precipitation. Total DNA was quantitated and 50 ng of DNA was analyzed in a PE7700 quantitative PCR reaction using a designed lacZ-specific primer/probe combination multiplexed with an 18S rRNA-specific primer/probe set. The lacZ probe sequence was 5′-6FAM-ACCCCGTACGTCTTCCCGAGCG-TAMRA-3′ (SEQ ID NO:4); the lacZ sense primer sequence was 5′-GGGATCTGCCATTGTCAGACAT-3′ (SEQ ID NO:5); and the lacZ antisense primer sequence was 5′-TGGTGTGGGCCATAATTCAA-3′ (SEQ ID NO:______). The 18S rRNA probe sequence was 5′-JOE-TGCTGGCACCAGACTTGCCCTC-TAMRA-3′ (SEQ ID NO:6); the 18S sense primer sequence was 5′-CGGCTACCACATCCAAGGAA-3′ (SEQ ID NO:7); and the 18S antisense primer sequence was 5′-GCTGGAATTACCGCGGCT-3′ (SEQ ID NO:8).

Each 25-μl PCR sample contained 2.5 μl (50 ng) of purified DNA, 900 nM of each primer, 50 nM of each probe, and 12.5 μl of 2× Perkin-Elmer Master Mix. Following a 2-min 50° C. incubation and 2-min 95° C. denaturation step, the samples were subjected to 40 cycles of 95° C. for 15 sec. and 60° C. for 1 min. Fluorescent intensity of each sample was detected automatically during the cycles by the Perkin-Elmer Applied Biosystem Sequence Detector 7700 machine. Each PCR run included the following: no-template control samples, positive control samples consisting of either amplicon DNA (for lacZ) or cellular genomic DNA (for 18S rRNA), and standard curve dilution series (for lacZ and 18S). Following the PCR run, “real-time” data were analyzed using Perkin-Elmer Sequence Detector Software version 1.6.3 and the standard curves. Precise quantities of starting template were determined for each titering sample and results were expressed as numbers of vector genomes per ml of original viral stock.

Western blot analysis: BHK cell monolayers (2×10⁶ cells) transfected with varying packaging components were lysed with RIPA buffer (150 mM NaCl, 1% NP-40, 0.5% DOC, 0.5% SDS, and 50 mM Tris-Cl, pH 8). Equal amounts of protein were electrophoretically separated on a 10% SDS-PAGE gel and transferred to a PVDF membrane. The resultant blot was incubated with an anti-VP16 monoclonal antibody (Chemicon, Inc.), and specific VP16 immunoreactive band visualized using an alkaline phosphatase-based chemiluminescent detection kit (ECL).

Cytotoxicity Assays: The effect of BAC-packaged HSVlac stocks prepared in the presence or absence of VP16 and/or vhs on cell viability was determined using a lactate dehydrogenase (LDH) release-based assay (Promega Corp., Madison, Wis.). Equivalent expression units of virus from each packaging sample were used to transduce 5×10³ NIH 3T3 cells in 96-well flat-bottomed culture dishes. Quantitation of LDH release was performed according to manufacturer's instructions. Viability data were represented as normalized cell viability index.

Stereotactic injections: Mice were anesthetized with Avertin at a dose of 0.6 ml per 25 g body weight. After positioning in an ASI murine stereotactic apparatus, the skull was exposed via a midline incision, and burr holes were drilled over the following coordinates (bregma, +0.5 mm; lateral −2.0 mm; and deep, −3.0 mm) to target infections to the striatum. A 33 GA steel needle was gradually advanced to the desired depth, and 3 μl (equivalent in vitro titer) HSVPrPUC/CMVegfp virus was infused via a microprocessor-controlled pump over 10 minutes (UltraMicroPump, World Precision Instruments, Sarasota Springs, Fla.). The injector unit was mounted on a precision small animal stereotaxic frame (ASI Instruments, Warren, Mich.) micromanipulator at a 90° angle using a mount for the injector. Viral injections were performed at a constant rate of 300 nl/min. The needle was removed slowly over an additional 10-minute period.

Tissue preparation and GFP visualization: Infected mice were anesthetized four days later, a catheter was placed into the left ventricle, and intracardiac perfusion was initiated with 10 ml of heparinized saline (5,000 U/L saline) followed by 60 ml of chilled 4% PFA. Brains were extracted and postfixed for 1-2 hours in 4% PFA at 4° C. Subsequently, brains were cryoprotected in a series of sucrose solutions with a final solution consisting of a 30% sucrose concentration (w/v) in PBS. Forty micron serial sections were cut on a sliding microtome (Micron/Zeiss, Thornwood, N.Y.) and stored in a cryoprotective solution (30% sucrose (w/v), 30% ethylene glycol in 0.1 M phosphate buffer (pH 7.2)) at −20° C. until processed for GFP visualization. Sections were placed into Costar net wells (VWR, Springfield, N.J.) and incubated for 2 hrs in 0.1 M Tris buffered saline (TBS) (pH 7.6). Upon removal of cryoprotectant, two additional 10 min washes in 0.1 M TBS with 0.25% Triton X-100 (Sigma, St. Louis, Mo.) were performed. Sections were mounted with a fine paint brush onto subbed slides, allowed to air dry, and mounted with an aqueous mounting media, Mowiol. GFP-positive cells were visualized with a fluorescent microscope (Axioskop, Zeiss, Thornwood, N.Y.) utilizing a FITC cube (Chroma Filters, Brattleboro, Vt.). All images used for morphological analyses were digitally acquired with a 3-chip color CCD camera at 200× magnification (DXC-9000, Sony, Montvale, N.J.).

Morphological analyses: Cell counts were performed on digital images acquired within 24 hrs of mounting. At the time of tissue processing coronal slices were stored serially in three separate compartments. All compartments were processed for cell counting and GFP(+) cell numbers reflect cell counts throughout the entire injection site. All spatial measurements were acquired using an image analysis program (Image-Pro Plus, Silver Spring, Md.) at a final magnification of 200×. Every section was analyzed using identical parameters in three different planes of focus throughout the section to prevent repeated scoring of GFP(+) cells. Each field was analyzed by a computer macro to count cells based on the following criteria: object area, image intensity (fluorescent signal) and plane of focus. Only cells in which the cell body was unequivocally GFP(+) and nucleus clearly defined were counted. Every section that contained a GFP(+) cell was counted. In addition, a watershed separation technique was applied to every plane of focus in each field to delineate overlapping cell bodies. The watershed method is an algorithm that is designed to erode objects until they disappear, then dilates them again such that they do not touch.

Statistical Analyses: Statistical analyses were carried out using one-way analyses of variance (ANOVA) with plasmid construct as the between-group variable. Two-way repeated measure analyses of variance (RMANOVA) were carried out using plasmid construct as the between-group variable and time interval as a within-group variable.

Results: Prior to the methods described herein, widespread use of helper virus-free HSV particles has been hampered by helper virus-mediated cytotoxicity associated with traditionally packaged amplicon stocks or by the low titers obtained from helper virus-free production methods. Helper virus-free methods of packaging hold the most promise as resultant stocks exhibit little or no cytotoxicity. As shown here, modifications to such packaging strategies could be made to increase viral titers.

We utilized both cosmid- and BAC-based methods of helper virus-free packaging previously described (Fraefel et al., J. Virol 70:719-7197, 1996; Stavropoulos and Strathdee, J. Virol. 72:7137-7143, 1998; and Saeki et al., Hum. Gene Ther. 9:2787-2794, 1998). The low titers observed for helper virus-free methods may be a result of the sub-optimal state of the HSV genome at the beginning of amplicon production, as the genome is without co-packaged viral regulators vhs and VP16. To determine if introduction of vhs into the packaging scheme could increase amplicon titers and quality, we cloned a genomic segment of the UL41 gene into pBluescript and added this plasmid (pBSKS(vhs)) to the co-transfection protocols to provide vhs in trans. The genomic copy of UL41 contained the transcriptional regulatory region and flanking cis elements believed to confer native UL41 gene expression during packaging. When pBSKS(vhs) was added to the packaging protocols for production of a β-galactosidase (lacZ)-expressing amplicon (HSVlac), a maximum of 10-fold enhanced amplicon expression titers was observed for both cosmid- and BAC-based strategies. As observed previously, the expression titers for HSVlac virus produced by the BAC-based method were approximately 500- to 1000-fold higher than stocks produced using the modified cosmid set. Even though titers were disparate between the differently prepared stocks, the effect of additionally expressed vhs on amplicon titers was analogous.

The punctate appearance of reporter gene product (pseudotransduction), a phenomenon associated with first-generation helper virus-free stocks, was substantially diminished in vitro when vhs was included in BAC-based packaging of a β-galactosidase-expressing (HSVlac) or an enhanced green fluorescent (GFP)-expressing virus (HSVPrPUC/CMVegfp). Pseudotransduction was not observed, as well, for cosmid-packaged amplicon stocks prepared in the presence of vhs. To assess the ability of the improved amplicon stocks to mediate gene delivery in vivo, BAC-packaged HSVPrPUC/CMVegfp virus prepared in the absence or presence of pBSKS(vhs) was injected stereotactically into the striata of C57BL/6 mice (see above). Four days following infection, animals were sacrificed and analyzed for GFP-positive cells present in the striatum. The numbers of cells transduced by HSVPrPUC/CMVegfp prepared in the presence of vhs were significantly higher than in animals injected with stocks produced in the absence of vhs. In fact, it was difficult to definitively identify GFP-positive cells in animals transduced with vhs(−) amplicon stocks.

The mechanism by which vhs expression resulted in higher apparent amplicon titers in helper virus-free packaging could be attributed to one or several properties of vhs. The UL41 gene product is a component of the viral tegument and could be implicated in structural integrity, and its absence could account for the appearance of punctate gene product material following transduction. For example, the viral particles may be unstable as a consequence of lacking vhs. Thus, physical conditions, such as repeated freeze-thaw cycles or long-term storage, may have led to inactivation or destruction of vhs-lacking virions at a faster rate than those containing vhs.

The stability of HSVPrPUC/CMVegfp packaged via the BAC method in the presence or absence of vhs was analyzed initially with a series of incubations at typically used experimental temperatures. Viral aliquots from prepared stocks of HSVPrPUC/CMVegfp were incubated at 4, 22, or 37° C. for periods up to three hours. Virus recovered at time points 0, 30, 60, 120, and 180 minutes were analyzed for their respective expression titer on NIH 3T3 cells. The rates of decline in viable amplicon particles, as judged by their ability to infect and express GFP, did not differ significantly between the vhs(+) and vhs(−) stocks. Another condition that packaged amplicons encounter during experimental manipulation is freeze-thaw cycling. Repetitive freezing and thawing of virus stocks is known to diminish numbers of viable particles, and potentially the absence of vhs in the tegument of BAC-packaged amplicons leads to sensitivity to freeze fracture. To test this possibility, viral aliquots were exposed to a series of four freeze-thaw cycles. Following each cycle, samples were removed and titered for GFP expression on NIH 3T3 cells as described previously. At the conclusion of the fourth freeze-thaw cycle, the vhs(−) HSVPrPUC/CMVegfp stock exhibited a 10-fold diminution in expression titers as opposed to only a 2-fold decrease for vhs(+) stocks. This observation suggests that not only do vhs(+) stocks have increased expression titers, but the virions are more stable when exposed to temperature extremes, as determined by repetitive freeze-thaw cycling.

The native HSV genome enters the host cell with several viral proteins besides vhs, including the strong transcriptional activator VP16. Once within the cell, VP16 interacts with cellular transcription factors and HSV genome to initiate immediate-early gene transcription. Under helper virus-free conditions, transcriptional initiation of immediate-early gene expression from the HSV genome may not occur optimally, thus leading to lower than expected titers. To address this issue, a VP16 expression construct was introduced into packaging cells prior to cosmid/BAC, amplicon, and pBSKS(vhs) DNAs, and resultant amplicon titers were measured. To achieve regulated expression a glucocorticoid-controlled VP16 expression vector was used (pGRE₅vp16).

The pGRE₅vp16 vector was introduced into the packaging cells 24 hours prior to transfection of the regular packaging DNAs. HSVlac was packaged in the presence or absence of vhs and/or VP16 and resultant amplicon stocks were assessed for expression titer. Some packaging cultures received 100-nM dexamethasone at the time of pGRE₅vp16 transfection to strongly induce VP16 expression; others received no dexamethasone. Introduction of pGRE₅vp16 in an uninduced (basal levels) or induced state (100 nM dexamethasone) had no effect on HSVlac titers when vhs was absent from the cosmid- or BAC-based protocol. In the presence of vhs, addition of pGRE5vp16 led to either a two- or five-fold enhancement of expression titers over those of stocks packaged with only vhs (cosmid- and BAC-derived stocks). The effect of “uninduced” pGRE₅vp16 on expression titers suggested that VP16 expression was occurring in the absence of dexamethasone. To examine this, Western blot analysis with a VP16-specific monoclonal antibody was performed using lysates prepared from BHK cells transfected with the various packaging components. Cultures transfected with pGRE₅vp16/BAC/pBSKS(vhs) in the absence of dexamethasone did show VP16 levels intermediate to cultures transfected either with BAC alone (lowest) or those transfected with pGRE₅vp16/BAC/pBSKS(vhs) in the presence of 100 nM dexamethasone (highest)(FIG. 4C). There was no difference in level of pGRE₅vp16-mediated expression in the presence or absence of BAC, nor did dexamethasone treatment induce VP16 expression from the BAC.

VP16-mediated enhancement of packaged amplicon expression titers could be due to increased DNA replication and packaging of amplicon genomes. Conversely, the additional VP16 that is expressed via pGRE₅vp16 could be incorporated into virions and act by increasing vector-directed expression in transduced cells. To test the possibility that VP16 is acting by increasing replication in the packaging cells, concentrations of vector genomes in BAC-derived vector stocks were determined. HSVlac stocks produced in the presence or absence of vhs and/or VP16 were analyzed using a “real-time” quantitative PCR method. The concentration of vector genome was increased two-fold in stocks prepared in the presence of VP16 and this increase was unaffected by the presence of vhs.

There is a possibility that addition of viral proteins, like vhs and VP16, to the packaging process may lead to vector stocks that are inherently more cytotoxic. The amplicon stocks described above were examined for cytotoxicity using a lactate dehydrogenase (LDH) release-based cell viability assay. Packaged amplicon stocks were used to transduce NIH 3T3 cells and 48 hours following infection, viability of the cell monolayers was assessed by the LDH-release assay. Amplicon stocks produced in the presence of vhs and VP16 displayed less cytotoxicity on a per virion basis than stocks packaged using the previously published BAC-based protocol (Stavropoulos and Strathdee, supra).

Significance: Wild-type HSV virions contain multiple regulatory proteins that prepare an infected host cell for virus propagation. These virally encoded regulators, which are localized to the tegument and nucleocapsid, include vhs and VP16, respectively. The UL41 gene-encoded vhs protein exhibits an essential endoribonucleolytic cleavage activity during lytic growth that destabilizes both cellular and viral mRNA species (Smibert et al., J. Gen. Virol. 73:467-470, 1992). Vhs-mediated ribonucleolytic activity appears to prefer the 5′ ends of mRNAs over 3′ termini, and the activity is specific for mRNA, as vhs does not act upon ribosomal RNAs (Karr and Read, Virology 264:195-204, 1999). Vhs also serves a structural role in virus particle maturation as a component of the tegument. HSV isolates that possess disruptions in UL41 demonstrate abnormal regulation of IE gene transcription and significantly lower titers than wild-type HSV-1 (Read and Frenkel, J. Virol. 46:498-512, 1983), presumably due to the absence of vhs activity. Therefore, because vhs is essential for efficient production of viable wild-type HSV particles, it likely plays a similarly important role in packaging of HSV-1-derived amplicon vectors.

The term “pseudotransduction” refers to virion expression-independent transfer of biologically active vector-encoded gene product to target cells (Liu et al., J. Virol. 70:2497-2502, 1996; Alexander et al. Human Gene Ther. 8:1911-1920, 1997. This phenomenon was originally described with retrovirus and adeno-associated virus vector stocks and was shown to result in an overestimation of gene transfer efficiencies. β-galactosidase and alkaline phosphatase are two commonly expressed reporter proteins that have been implicated in pseudotransduction, presumably due to their relatively high enzymatic stability and sensitivity of their respective detection assays (Alexander et al., supra). Stocks of β-galactosidase expressing HSVlac and GFP-expressing HSVPrPUC/CMVegfp exhibited high levels of pseudotransduction when packaged in the absence of vhs. Upon addition of vhs to the previously described helper virus-free packaging protocols, a 10-fold increase in expression titers and concomitant decrease in pseudotransduction were observed in vitro.

Vhs-mediated enhancement of HSV amplicon packaging was even more evident when stocks were examined in vivo. GFP-expressing cells in animals transduced with vhs(+) stocks were several hundred-fold greater in number than in animals receiving vhs(−) stocks. This could have been due to differences in virion stability, where decreased particle stability could have led to release of co-packaged reporter gene product observed in the case of vhs(−) stocks. Additionally, the absence of vhs may have resulted in packaging of reporter gene product into particles that consist of only tegument and envelope (Rixon et al., J. Gen. Virol. 73:277-284, 1992). Release of co-packaged reporter gene product in either case could potentially activate a vigorous immune response in the CNS, resulting in much lower than expected numbers of vector-expressing cells.

Pre-loading of packaging cells with low levels of the potent HSV transcriptional activator VP16 led to a 2- to 5-fold additional increase in amplicon expression titers only in the presence of vhs for cosmid- and BAC-based packaging systems, respectively. This observation indicates the transactivation and structural functions of VP16 were not sufficient to increase viable viral particle production when vhs was absent, and most likely led to generation of incomplete virions containing amplicon genomes as detected by quantitative PCR. When vhs was present for viral assembly, however, VP16-mediated enhancement of genome replication led to higher numbers of viable particles formed. Quantitative PCR analysis of amplicon stocks produced in the presence of VP16 and vhs showed that viral genomes were increased only 2-fold while expression titers were increased 5-fold over stocks produced in the presence of vhs only. This result suggests that a portion of the effect related to VP16-mediated enhancement of genome replication while the additional ˜2-fold enhancement in expression titers may be attributed to the structural role of VP16. The effect of VP16 on expression titers was not specific to amplicons possessing the immediate-early 4/5 promoter of HSV, as amplicons with other promoters were packaged to similar titers in the presence of VP16 and vhs.

VP16 is a strong transactivator protein and structural component of the HSV virion (Post et al., Cell 24:555-565, 1981). VP16-mediated transcriptional activation occurs via interaction of VP16 and two cellular factors, Oct-1 (O'Hare and Goding, Cell 52:435-445, 1988; Preston et al., Cell 52:425-434, 1988; Stern et al., Nature 341:624-630, 1989) and HCF (Wilson et al., Cell 74:115-125, 1993; Xiao and Capone, Mol. Cell Biol. 10:4974-4977, 1990) and subsequent binding of the complex to TAATGARAT elements found within HSV IE promoter regions (O'Hare, Semin. Virol. 4:145-155, 1993. This interaction results in robust up-regulation of IE gene expression. Neuronal splice-variants of the related Oct-2 transcription factor have been shown to block IE gene activation via binding to TAATGARAT elements (Lillycrop et al., Neuron 7:381-390, 1991) suggesting that cellular transcription factors may also play a role in limiting HSV lytic growth.

The levels of VP16 appear to be important in determining its effect on expression titers. Low, basal levels of VP16 (via uninduced pGRE₅vp16) present in the packaging cell prior to introduction of the packaging components induced the largest effect on amplicon expression titers. Conversely, higher expression of VP16 (via dexamethasone-induced pGRE₅vp16) did not enhance virus production to the same degree and may have, in fact, abrogated the process. The presence of glucocorticoids in the serum components of growth medium is the most likely reason for this low-level VP16 expression, as charcoal-stripped sera significantly reduces basal expression from this construct. Perhaps only a low level or short burst of VP16 is required to initiate IE gene transcription, but excessive VP16 leads to disruption of the temporal progression through the HSV lytic cycle, possibly via inhibition of vhs activity. Moreover, evidence has arisen to suggest vhs activity is downregulated by interaction with newly synthesized VP16 during the HSV lytic cycle, thereby allowing for accumulation of viral mRNAs after host transcripts have been degraded (Schmelter et al., J. Virol. 70:2124-2131, 1996; Smibert et al., J. Virol. 68:2333-2346, 1994; Lam et al., EMBO J. 15:2575-2581, 1996). Therefore, a delicate regulatory protein balance may be required to attain optimal infectious particle propagation. Additionally, the 100-nM dexamethasone treatment used to induce VP16 expression may have a deleterious effect on cellular gene activity and/or interfere with replication of the OriS-containing amplicon genome in packaging cells. High levels of dexamethasone have been shown previously to repress HSV-1 OriS-dependent replication by an unknown mechanism Hardwicke and Schaffer, J. Virol. 71:3580-3587, 1997). Inhibition of OriS-dependent replication does not appear to be responsible for our results, however, since quantitative PCR analysis of amplicon stocks produced in the presence and absence of dexamethasone indicated no change in genome content as a function of drug concentration. It is interesting to note that amplicon stocks were prepared in the presence of hexamethylene bisacetamide (HMBA). HMBA has been shown to compensate for the absence of VP16, thus leading to the transactivation of immediate early gene promoters (McFarlane et al., J. Gen. Virol. 73:285-292, 1992. In the absence of HMBA pre-loading a packaging cell with VP16 could impart an even more dramatic effect on titers.

Ectopic expression of vhs and VP16 did not lead to amplicon stocks that exhibited higher cytotoxicity than helper virus-free stocks prepared in the traditional manner when examined by an LDH-release assay. Stocks prepared by the various methods were equilibrated to identical expression titers prior to exposure to cells. The heightened cytotoxicity in stocks produced in the absence of vhs and/or VP16 may reflect that larger volumes of these stocks were required to obtain similar expression titers as the vhs/VP16-containing samples or the levels of defective particles in the former may be significantly higher. Contaminating cellular proteins that co-purify with the amplicon particles are most likely higher in concentration in the traditional stocks, and probably impart the higher toxicity profiles observed.

### Example 11

### Herpes virus Amplicon Particles in the Treatment of Hematologic Malignancies

The experiments described below were designed to test viral-based amplicons as therapeutic agents for hematologic (and other types of) malignancies. We transduced tumor cells ex vivo with various HSV-based amplicons that encode different co-stimulatory molecules, such as B7.1 (also known as CD80) and CD40L (also known as CD154). In addition, we tested two HSV amplicon stocks: one packaged using a helper virus (manufactured via a replication-defective helper virus deleted in HSV ICP4) and one prepared, helper virus-free, using a bacterial artificial chromosome (BAC). Stocks packaged in either way were prepared to express either B7.1 or CD40L. The helper virus-containing and the helper virus-free stock were tested for their ability to transduce freshly isolated human B cell chronic lymphocytic leukemia (CLL) cells, to function as antigen-presenting cells, to stimulate T cell proliferative responses and cytokine release, and to affect MHC-I expression in transduced target CLL cells.

Using CLL cells, we found that: (1) both helper virus-containing and helper virus-free virus stocks are able to transduce primary human leukemia cells at high efficiencies, and (2) cells transduced with helper virus-containing amplicon were less efficient as APCs, and thus not as desirable as helper virus-free preparations for use in immunotherapies. The disadvantages of using a helper virus-containing preparation arise from the transcription of certain genes within the HSV genome, which is delivered largely intact into the host cell with the helper virus. More specifically, we found: (1) loss of MHC-I on cells transduced with helper virus-containing HSV amplicon stocks (this is likely to be mediated by the ICP-47 gene product that is introduced with the helper virus) and (2) increased cytotoxicity in cells transduced by the helper virus-containing amplicon stock. With respect to (1), loss of MHC-I hampers CD8-mediated CTL activity and results in a loss of the ability to kill target tumor cells. With respect to (2), the increased cytotoxicity in CLL cells is most likely related to the introduction of pro-apoptotic genes mediated by the helper virus. Due to these issues (inherent immunosuppression and cytotoxicity), helper virus-free amplicon preparations emerge as a superior choice for developing immunotherapies to treat any number of infectious diseases and cancers (including chronic lymphocytic leukemia).

Cell culture: Samples of blood (10 ml each) were obtained from eight patients with an established diagnosis of CLL. Peripheral blood lymphocytes (PBL) were isolated by density gradient centrifugation on Ficoll-Paque™ Plus (Amersham Pharmacia Biotech AB, Uppsala, Sweden). More than 97% of purified PBL stained positive for CD19 by flow-cytometry. Allogeneic T cells were purified from healthy donor PBL through a T cell enrichment column (R&D Systems, Minneapolis, Minn.). More than 97% of the purified lymphocytes obtained from the T cell column were CD3 positive by flow cytometry. Both CLL cells and T cells were maintained in RPMI supplemented with 10% human AB serum. Baby hamster kidney (BHK) and RR1 cell lines were maintained as described in Kutubuddin et al. (Blood 93:643-654, 1999). The NIH 3T3 mouse fibroblast cell line was originally obtained from the American Type Culture Collection (Manassas, Va.) and maintained in Dulbecco's modified Eagle medium (DMEM) plus 10% fetal bovine serum (FBS).

Amplicon Construction: Coding sequences for E. coli β-galactosidase and human B7.1 (CD80) were cloned into the polylinker region of the pHSVPrPUC plasmid (Geller et al., Proc. Natl. Acad Sci. USA 87:8950-8954, 1990) as described by Kutubuddin et al. (Blood 93643-654, 1999). Murine CD40L (CD154; kindly provided by Dr. Mark Gilber, Immunex Corp.) was cloned into the BamHI and EcoRI sites of the pHSVPrPUC amplicon vector.

Helper virus-based amplicon packaging: Amplicon DNA was packaged into HSV-1 particles by transfecting 5 μg of plasmid DNA into RR1 cells with Lipofectamine as recommended by the manufacturer (GIBCO-BRL). Following incubation for 24 hours, the transfected monolayer was superinfected with the HSV strain 17-derived IE3 deletion mutant virus D30EBA (Paterson and Everett, J. Gen. Virol. 71:1775-1783, 1990) at a multiplicity of infection (MOI) of 0.2. Once cytopathic changes were observed in the infected monolayer, the cells were harvested, freeze-thawed, and sonicated using a cup sonicator (Misonix, Inc.). Viral supernatants were clarified by centrifugation at 5000×g for ten minutes prior to repeat passage on RR1 cells. This second-viral passage was harvested as above and concentrated for two hours by ultracentrifugation on a 30% sucrose cushion as described by Federoff (In Cells: A Laboratory Manual, Spector and Leinwand, Eds., Cold Spring Harbor Laboratory Press, Cold Spring Harbor, N.Y., 1997). Viral pellets were resuspended in PBS (Ca²⁺ and Mg²⁺ free) and stored at −80° C. for future use.

Helper virus-free amplicon packaging (HF-HSV): Amplicon stocks were also prepared using a modified helper virus-free packaging method. The packaging system utilizes a bacterial artificial chromosome (BAC; kindly provided by C. Strathdee) that contains the HSV genome without its cognate pac signals as a co-transfection reagent with amplicon DNA. Because the amplicon vector possesses pac signals, only the amplicon genome is packaged. Briefly, on the day prior to transfection, 2×10⁷ BHK cells were seeded in a T-150 flask and incubated overnight at 37° C. The day of transfection, 1.8 ml Opti-MEM (Gibco-BRL, Bethesda, Md.), 25 μg of pBAC-V2 DNA (Stavropoulos and Strathdee, supra), 7 μg of pBS(vhs), and 3.6 μg amplicon vector DNA were combined in a sterile polypropylene tube. Seventy microliters of Lipofectamine Plus reagent (Gibco-BRL) were added over a period of 30 seconds to the DNA mix and allowed to incubate at 22° C. for 20 minutes. In a separate tube, 100 μl Lipofectamine (Gibco-BRL) was mixed with 1.8 ml Optim-MEM and also incubated at 22° C. for 20 minutes. Following the incubations, the contents of the two tubes were combined over a period of 30 seconds, and incubated for an additional 20 minutes at 22° C. During this second incubation, the media in the seeded T-150 flask was removed and replaced with 14 ml Opti-MEM. The transfection mix was added to the flask and allowed to incubate at 37° C. for five hours. The transfection mix was then diluted with an equal volume of DMEM plus 20% FBS, 2% penicillin/streptomycin, and 2 mM hexamethylene bis-acetamide (HMBA), and incubated overnight at 34° C. The following day, media was removed and replaced with DMEM plus 10% FBS, 1% penicillin/streptomycin, and 2 mM HMBA. The packaging flask was incubated an additional three days before virus was harvested and stored at −80° C. until purification. Viral preparations were subsequently thawed, sonicated, clarified by centrifugation, and concentrated by ultracentrifugation through a 30% sucrose cushion. Viral pellets were resuspended in 100 μl PBS (Ca²⁺ and Mg²⁺ free) and stored at −80° C. for future use.

Virus Titering: Helper virus-containing stocks were titered for helper virus by standard plaque assay methods (Geschwind et al., Brain Res. Mol. Brain Res. 24:327-335, 1994). Amplicon titers for both helper virus-based and helper-free stocks were determined as follows. NIH 3T3 cells were plated in a 24-well plate at a density of 1×10⁵ cells/well and infected with the virus. Twenty-four hours after viral infection, the monolayers were washed twice in PBS and either fixed with 4% paraformaldehyde and stained by X-gal histochemistry (HSVlac; 5 mM potassium ferricyanide; 5 mM potassium ferrocyanide; 0.02% NP40; 0.01% sodium deoxycholic acid; 2 mM MgCl₂; and 1 mg/ml X-gal dissolved in PBS) or harvested for total DNA using lysis buffer (100 mM NaCl, 10 mM Tris, pH 8.0, 25 mM EDTA, 0.5% SDS) followed by phenol/chloroform extraction and ethanol precipitation. Real-time quantitative PCR was performed on duplicate samples using primers corresponding to the β-lactamase gene present in the amplicon plasmid, according to Bowers et al. (Mol. Ther. 1:294-299, 2000). Total DNA was quantitated and 50 ng of DNA was analyzed in a PE7700 quantitative PCR reaction using a designed β-lactamase-specific primer/probe combination multiplexed with an 18S rRNA-specific primer/probe set. The β-lactamase probe sequence was 5′-CAGGACCACTTCTGCGCTCGGC-3′ (SEQ ID NO:9); the β-lactamase sense primer sequence was 5′-CTGGATGGAGGCGGATAAAGT-3′ (SEQ ID NO:10); and the β-lactamase antisense primer sequence was 5′-TGCTGGCACCAGACTTGCCCTC-3′ (SEQ ID NO:11). The 18S rRNA probe sequence was 5′-TGCTGGCACCAGACTTGCCCTC-3′ (SEQ ID NO:12); the 18S sense primer sequence was 5′-CGGCTACCACATCCAAGGAA-3′ (SEQ ID NO:13); and the 18S antisense primer sequence was 5′-GCTGGAATTACCGCGGCT-3′ (SEQ ID NO:14). Helper virus titers (pfu/ml), amplicon expression titers (bfu/ml), and amplicon transduction titers (TU/ml) obtained from these methods were used to calculate amplicontiter and thus standardize experimental viral delivery. Amplicon titers of the various virus preparations ranged from 4-5×10⁸ bfu/ml while helper titers were in the range of 5-15×10⁷ pfu/ml.

Mixed lymphocyte tumor reaction (MLTR) assay: CLL cells were transduced with equal transduction units of helper virus-containing or helper virus-free amplicon stocks, were irradiated (20 Gy), and were used as stimulators (2.5 or 5×10⁴ cells/well) with allogeneic normal donor T cells (2×10⁵ cells in a final volume of 200 μl) in 96-well flat-bottom plates. All cultures were performed in triplicate. The cells were incubated 5 days at 37° C. in 5% CO₂. Cells were pulsed with 1 μCi (³H)-thymidine for the last 18 hours of the culture period before being transferred onto a glass fiber filter and radioactive counts measured by liquid scintillation counting. To determine the involvement of Signal One, CLL cells were infected with equivalent transduction units of HSVlac, HSVB7.1, hf-HSVlac, or hf-HSVB7.1 and were used as stimulators as described above with or without phorbol 12-myristate 13-acetate (PMA) added to a final concentration of 10 ng/ml.

ELISA for IL-2 and γ-interferon: Culture supernatant (50 μl) from every well of the MLTR plate was collected on day 4 prior to adding (³H)-thymidine and used in a standard sandwich ELISA (R&D Systems) according to manufacturer recommendations.

Cytotoxic T lymphocyte (CTL) Assay: T cells purified from normal donor peripheral blood mononuclear cells (PBMC) were incubated with uninfected irradiated CLL cells, helper virus-free HSVlac-, or helper virus-free HSVCD40L-infected CLL cells at a ratio of 4:1 and incubated for six days. A cytotoxicity assay was performed by incubating primed T cells with 1×10^{4 51}Cr-labeled CLL cells in a V-shaped 96-well plate at varying effector:target ratios. Spontaneous release was measured by incubating ⁵¹Cr-labeled CLL cells alone while maximum release was calculated by lysing the cells with 2% Triton-X. After a six-hour incubation, supernatant was collected and radioactivity was measured using a γ-counter (Packard Instrument). Mean values were calculated for the triplicate wells and the results are expressed as % specific lysis according to the formula: experimental counts−spontaneous counts/total counts−spontaneous counts×100.

Results

HSV amplicon-mediated gene transfer into CLL cells. The utility of HSV-based amplicon vectors for transduction of CLL cells was examined according to the methods described above. HSV amplicon vectors encoding β-galactoside, CD80 (B7.1) or CD154 (CD40L) were packaged using either a standard helper virus (designated HSVlac, HSVB7.1 and HSVCD40L) or a helper virus-free method (designated hf-HSVlac, hf-HSVB7.1 and hf-HSVCD40L).

CLL cells were isolated by density gradient centrifugation and ≧97% of the cells stained for CD19, a cell surface marker for B lymphocytes. The cells were transduced with either HSVlac, HSVB7.1, hf-HSVlac, or hf-HSVB7.1. X-gal histochemistry was performed to detect the β-galactosidase (lacZ) transgene product expressed by HSVlac and hf-HSVlac, while fluorescence activated cell sorting (FACS) analyses were performed on CLL cells transduced with equivalent transduction units of HSVB7.1 and hf-HSVB7.1 (FIG. 10). More than 70% of the cells stained for either lacZ or B7.1 expression at an MOI of 1.0. In agreement with previous studies using HSVlac, expression levels of β-galactosidase peaked at 2-3 days and persisted for up to 7 days post-infection. Hence, both helper virus-containing and helper virus-free amplicon preparations appear to be effective for gene transfer into CLL cells.

Effect of helper virus on host cell MHC-I expression. Although both vector preparations were able to drive high-level expression of B7.1 in CLL cells, it was possible that helper virus-containing amplicon preparations disrupted MHC I-mediated antigen presentation. ICP47, a gene present in the D30EBA helper virus, encodes a protein that blocks TAP-1 mediated peptide loading into MHC I. Expression of such an immunosuppressive activity would reduce the utility of HSV amplicon vectors for immunotherapeutic strategies. To examine this possibility, CLL cells were transduced with HSVB7.1 or hf-HSVB7.1 and examined by flow-cytometry for levels of B7.1 and MHC I expression.

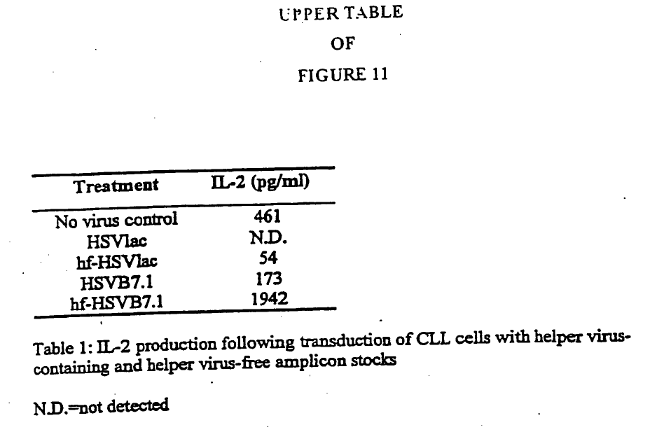

Significant down-regulation of MHC I in CLL cells transduced with HSVB7.1 was observed compared to MHC I expression in uninfected cells (FIG. 11). In contrast, transduction with hf-HSB7.1 resulted in high levels of B7.1 expression and maintenance of MHC I surface expression on B7.1-transduced cells. These data highlight the role of HSV-encoded factors in modulation of host immunity and underscore a fundamental difference in the immunotherapeutic potential between helper virus-based and helper virus-free amplicon preparations.

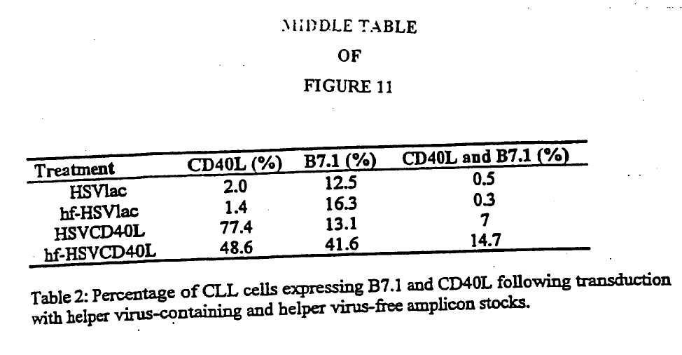

Allogeneic T cell activation by HSV amplicon-transduced CLL cells. To assess functional differences in antigen presentation following transduction with helper virus-containing or helper virus-free amplicon stocks, the effects of B71. transduction on the ability of CLL cells to stimulate T cell proliferation in an allogeneic mixed leukocyte tumor reaction (MLTR) were analyzed. CLL cells were transduced with either HSVlac, HSVB7.1, hf-HSVlac, or hf-HSVB7.1 and transduced cells served as stimulators in an allogeneic MLTR using T cells from a normal donor. hf-HSVB7.1-transduced CLL cells were able to directly stimulate T cell proliferation (FIG. 12). In spite of amplicon-directed expression of B7.1 on at least 70% of the CLL cells, HSVB7.1 -transduced CLL cells failed to elicit a T cell proliferative response, suggesting that the antigen presenting capacity of the infected CLL cells had been seriously impaired. This could have occurred through the loss of MHC I expression (as shown in FIG. 11) or through some other mechanism mediated by the helper virus. Phorbol 12-myristate 13-acetate (PMA) was used to provide an extrinsic “signal one” to potentially compensate for the adverse effect elicited by the helper virus on CLL cells, thereby allowing transduced B7.1 to elicit a co-stimulatory signal to T cells. Provision of extrinsic Signal One by PMA resulted in significant proliferation in HSVB7.1-infected CLL cells (relative to non-transduced or HSVlac-transduced CLL cells). PM treatment also augmented proliferation in hf-HSVB7.1-transduced CLL cells, suggesting that the full potential of T cell activation by these transduced cells was not fully achieved by helper virus-free vector delivery alone.

Another correlate to T cell activation relates to induction of IL-2 secretion. Supernatants collected from the MLTR samples described above were analyzed using an IL-2 ELISA. IL-2 levels were highest when hf-HSVB7.1-transduced CLL cells were utilized as T cell stimulators (the uppermost Table in FIG. 11) as compared to HSVB7.1 or HSVlac-transduced cells. In other MLTR assays using HSVB7.1-transduced CLL cells, IL-2 secretion was dependent on provision of Signal One via PMA, as was observed with PMA-mediated rescue of T cell stimulators.

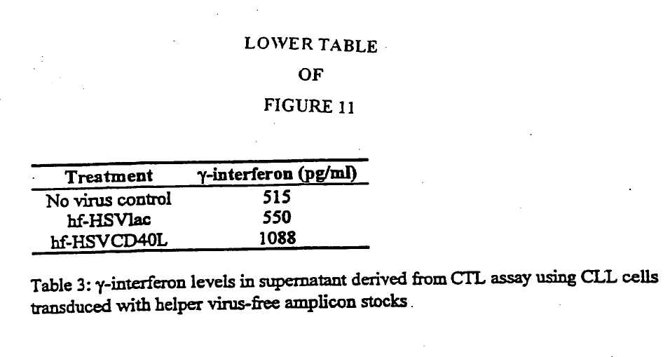

Up-regulation of co-stimulatory molecules on CLL cells transduced by HSV amplicons. Engagement of the CD40 receptor on APCs is a critical step in the initiation of an immune response. Up-regulation of costimulatory molecules on CLL cells induced by CD40 receptor signaling correlates with a cell's ability to function as an APC (van Kooten et al., Curr. Opin. Immunol. 9:330-337, 1997; Gruss et al., Leuk. Lymphoma 24:393-422, 1997). We selected endogenous B7.1 expression as a surrogate marker for the morphologic changes induced by CD40 receptor engagement in CLL cells. To test for paracrine and autocrine induction of B7.1, CLL cells were transduced with either hf-HSVCD40L or hf-HSVlac, incubated for six days and subsequently analyzed for expression of endogenous B7.1. As shown in FIG. 13, transduction with hf-HSVCD40L resulted in up-regulation of B7.1 on CLL cells as compared to untransduced and hf-HSVlac transduced cells.

The percentage of CLL cells expressing B7.1, CD40L, or both, was quantitated by two-color flow cytometry (the middle Table in FIG. 11). Although infection of CLL cells with HSVCD40L resulted in more than 70% of the cells expressing CD40L, the percentage of cells expressing endogenous B7.1 did not increase over background levels observed in cells transduced with control vector. CLL cells infected with hf-HSVCD40L exhibited a marked enhancement of B7.1 expression. The discrepancy at the level of endogenous B7.1 expression between CLL cells transduced with HSVCD40L and hf-HSVCD40L cannot be attributed to different efficiencies of infectivity as both groups expressed similar levels of CD40L. Similar experiments using CD19 expression as an endogenous cell marker confirmed an inverse relationship between surface CD19 expression and CD40L expression in cells transduced with helper virus-containing HSVCD40L, but not in cells transduced with hf-HSVCD40L. These data suggested that transduction with HSVCD40L resulted in a decrease in expression level of endogenous B7.1

Subsequently, the ability of CLL cells transduced by CD40L to serve as stimulators in an allogeneic MLTR was examined. CLL cells were transduced with hf-HSVlac, hf-HSVCD40L, HSVlac, or HSVCD40L and incubated for 4-6 days to allow for up-regulation of co-stimulatory molecules and then used as stimulators in an allogeneic MLTR. Although similar levels of CD40L expression were observed following transduction with either HSVCD40L or hf-HSVCD40L, cells transduced with hf-HSVCD40L were more potent T cell stimulators than those transduced with HSVCD40L or control vectors.

hf-HSV amplicon transduced CLL stimulate allogeneic CTL. Since the goal of immune therapy is to generate tumor-specific CTL, and in view of the data above showing superiority of helper virus-free stock, we tested the capacity of allogeneic T cells to elicit a cytotoxic response against CLL cells transduced with hf-HSVCD40L. T cells purified from normal donor peripheral blood mononuclear cells (PBMC) were incubated for six days with non-transduced/irradiated CLL cells, hf-HSVlac-, or hf-HSVCD40L-transduced CLL cells. A cytotoxicity assay was performed by incubating primed T cells with ⁵¹Cr-labeled CLL cells at varying effector to target ratios. Significantly higher CTL activity was generated by priming with hf-HSVCD40L-transduced CLL cells compared to control or hf-HSVlac-transduced cells. As another index of cytolytic T cell activation, we measured levels of gamma-interferon secretion. High levels of IFN-gamma were secreted by hf-HSVCD40L-transduced CLL stimulated T cells as detected by ELISA (the lower Table in FIG. 11), suggesting that helper virus-free amplicon stocks can effectively transduce CLL cells to serve as tumor vaccines.

DCs pulsed with CTL peptide epitopes derived from tumor antigens or transduced with adenoviral vectors that direct expression of tumor antigens have been shown to elicit antitumor CTL activity. However, each of these methods has limitations. For example, to use peptides for tumor immunotherapy, one would have to recognize CTL epitopes for tumor antigens in multiple HLA types and, with adenoviral vectors, the viral gene products expressed in transduced cells can lead to anti-vector immunity, which would preclude multiple immunizations.

### Example 12

### LIGHT, a TNF Family Member Enhances the Antigen Presenting Capacity of Chronic Lymphocytic Leukemia and Stimulates Autologous Cytolytic T Cells

CLL B cells possess the ability to process and present tumor antigens, but lack expression of costimulatory molecules, rendering them inefficient effectors of T-cell activation. We previously demonstrated that helper virus-free preparations of Herpes Simplex Virus (HSV) amplicon vectors encoding CD40L efficiently transduce CLL B cells and render them capable of eliciting specific anti-tumor T-cell responses (Tolba et al., Blood 98:287-295,2001). LIGHT (TNFSF14), a member of the TNF superfamily, represents a strong candidate molecule as it efficiently activates T cells as well as antigen-presenting cells (APC). We employed an HSV amplicon vector expressing human LIGHT (hf-HSVLIGHT) to transduce CLL B cells and compared the immunomodulatory function and T-cell activation by hf-HSV-LIGHT to that of the previously described CD40L-expressing amplicon (hf-HSVCD40L). hf-HSVLIGHT transduction induced expression of endogenous B7.1, B7.2 and ICAM.1, albeit to a lesser degree than observed in response to CLL B cells transduced with hf-HSV-CD40L. hf-HSVLIGHT enhanced antigen-presenting capacity of CLL B cells and stimulated T cell proliferation in an allogeneic mixed lymphocyte tumor reaction (MLTR) through a dual mechanism: a) indirectly through induction of native B7.1/B7.2 and b) directly via stimulation of Hve-A receptor on T cells. Finally, hf-HSVLIGHT transduced CLL B cells successfully stimulated outgrowth of autologous cytotoxic T-lymphocytes in vitro. These data suggest that hf-HSVLIGHT transduction may be useful for induction of immune responses to CLL and other B-cell lymphoid malignancies.

### Example 13

### HSV Amplicon-Mediated Neurotrophin-3 Expression Protects Murine Spiral Ganglion Neurons from Cisplatin-Induced Damage

In the paragraph that follows, we provide a summary of this study. We then describe the way our procedures were carried out and, following that, describe the results.

Ototoxicity is a major dose-limiting side effect of cisplatin (DPP) administration due to its propensity to induce destruction of hair cell and neurons in the auditory system. Previous studies demonstrated that TrkC-expressing spiral ganglion neurons (SGNs) are protected from the cytotoxic effects of DDP by localized delivery of the trophic factor neurotrophin-3 (NT-3). Successful in vivo implementation of such a therapy requires the development of an efficient gene delivery vehicle for expression of NT-3 within the cochlea. To this end, we constructed an HSV amplicon vector that expressed a c-Myc-tagged NT-3 chimera (HSVnt-3myc). Helper virus-free vector stocks were initially evaluated in vitro for their capacity to direct expression of NT-3 mRNA and protein. Transduction of cultured murine cochlear explants with HSVnt-3myc resulted in production of NT-3 mRNA and protein up to 3 ng/ml as measured over a 48-hour period in culture supernatants. To determine whether NT-3 overexpression could abrogate DDP toxicity, cochlear explants were transduced with HSVnt-3myc or a murine intestinal alkaline phosphatase-expressing control vector, HSVmiap, and then exposed to cisplatin. HSVnt-3myc-transduced cochlear explants harbored significantly greater numbers of surviving SGNs than those infected with control virus. These data demonstrate that amplicon-mediated NT-3 transduction can attenuate the ototoxic action of DDP on organotypic culture. The potency of NT-3 in protecting SGNs from degeneration indicates that in vivo neurotrophin-based gene therapy may be useful for the prevention and/or treatment of hearing disorders.

Construction of HSV amplicon vectors. The PBJ-T-NT3myc plasmid (kindly provided by Dr. Eric Shooter, Stanford University) contained the 800-bp NT-3myc fragment. To construct pHSVnt-3myc, the CMV promoter was cloned into the NotI and HindIII sites of the pHSVminOriS_{mc} parent amplicon vector (kindly provided by Dr. K. Maguire-Zeiss), and the NO-3myc fragment from pBJ-5-NT-3myc was subcloned into the pHSVCM-VminOriS_{mc} vector with blunt ends. The control vector lacked the NT-3myc fragment and contained only the 1.7-kb encoding fragment of murine alkaline phosphatase (MIAP) cDNA.

Helper virus-free packaging and viral titering. Twenty-five micrograms of pBAC-V2DNA, 7 μg of amplicon vector DNA were combined and transfected into 2×10⁷ BHK cells with Lipofectamine Plus reagent (Gibco BRL, Bethesda, Md.) in Opti-MEM (Gibco BRL) as previously described (Bowers et al., Gene Ther. 8:111-120, 2001). The virus was harvested, concentrated, resuspended in PBS, and stored at −80° C. until use. For transduction titers, 50 ng of DNA from infected 3T3 cells was analyzed in a Perkin-Elmer 7700 quantitative PCR using a designed amplicon-specific primer/probe combination multiplexed with an 18S rRNA-specific primer/probe set (Bowers et al., Mol. Ther. 1:294-299, 2000). Following the PCR run, “real-time” data were analyzed using Perkin-Elmer Sequence Detector Software version 1.6.3 and standard curves. Precise starting quantities were determined for each tittering sample and results were expressed as numbers of vector genomes per milliliter of original viral stock.

Culture of cochlear explants, transduction with HSV amplicon vectors, and cisplatin administration. Day 3 postnatal C57BL/6 mouse pups were sacrificed by rapid decapitation under deep halothane anesthesia and the heads were sterilized by dipping in 70% ethanol. An incision was made along the midline, and the bony-cartilaginous cochlear capsule was separated from the skull. After dissection, the spiral ligament and stria vascularis tissue were stripped away from the organ of Corti and five cochlear explants were put into 30-mm-diameter, 0.4-μm culture plate inserts (Millipore, Bedford, Mass.) coated with rat-tail collagen Type I (Sigma). The cochlear explants were cultured in serum-free DMEM/F12 medium supplemented with 100 units/ml penicillin, 30 mM glucose, 2 mM glutamine and incubated in 5% CO₂ with 95% O₂ at 37° C. Following 48 hours of culture, the tissues were infected with HSVnt-3myc (2.7×10⁵ transduction units; TU) and HSVmiap (2.7×10⁵ TU) virus stock at 37° C. for one hour, and then the media were changed to remove the virus. Forty-eight hours after infection, cisplatin (Bristol-Myers Squibb) was added into the media at various concentrations (0, 4, 6, 8 μg/ml) for an additional 96 hours of incubation before the cochlear explants were fixed as described in detail below.

ELISA. The media from cultured cochlear explants after 48 hours of HSVnt-3myc transduction were collected and stored at −80° C. The level of NT-3 secretion was quantified by using a two-site immunoassay. Blocking solution, wash buffer, and tetramethylbenzidine peroxidase-developing substrate were used (Promega). ELISA plates (Immobilon, Nunc) were coated with anti-human NT-3 pAB (1:500) in carbonate buffer (pH 9.7) and incubated overnight at 4° C. (NT-3 ELISA kit; Promega), followed by incubation of samples and detection of NT-3 by using anti-NT-3 mAb (1:4000) and anti-mouse IgG, HRP conjugate. The data analysis was performed on at least three independent experiments. The level of NT-3 production was calculated according to the standard curve performed on the same plate.

Reverse transcription polymerase chain reaction. Under sterile and RNase-free conditions, five cochlear explants of each group, 2 days after virus infection, were homogenized and solubilized in 400 μl TRIZOL reagent (Life Technologies, Gaithersburg, Md.) and total RNA was obtained according to the manufacturer's instructions. Total RNA was resuspended in 30 μl RNase-free water and stored at −80° C. RNA reverse transcription was performed with oligo(dT) (10 μM final concentration) in transcription buffer (50 mMKCl, 10 mM Tris-HCl, pH 9.0, 1.5 mM MgCl₂) containing 20 units of RNasin (Life Technologies), 1 mM dNTP, and 50 units of AMV reverse transcriptase (Life Technologies). Reaction conditions were 10 min at 72° C., 40 min at 40° C., and 30 min at 37° C. after adding RNase inhibitor. PCR amplifications were performed with 50-μl reaction volumes containing 10 μM oligonucleotide, 6 mM MgCl₂, and 2 units of Taq polymerase (Life Technologies) for 40 cycles; denaturation for 30 s at 94° C., annealing for 30 s at 61° C., and extension for 72 S at 72° C. The sense oligonucleotide primer, 5′-ATGAAACGAGGTGTAAAGAAGC-3′, began at nucleotide 575 in the rat NT-3 sequence, and the antisense oligonucleotide primer, 5′-CTGATGAGCTTCTGCTCGCC-3′, ended at nucleotide 797 in NT-3-myc epitope sequence. The “housekeeping gene” hypoxanthine-guanine phosphoribosyl transferase (HPRT) was used as the internal control. HPRT-specific primers were generated based upon published sequences from the GenEMBL database (HPRT, X62085). The sense oligonucleotide primer, 5′-CTGACCTGCTGGATTACATTA-3′, and the antisense oligonucleotide primer, 5′-CCACTTTCGCTGATGACACAA-3′, amplified a 416-bp fragment (Tokuyama et al., Brain Res. Brain Res. Protocols 4:407-414, 1999).

Western blot analysis. Cochlear explants of each group, two days after virus infection, were lysed with RIPA buffer (150 mM NaCl, 1% NP-40, 0.5% DOC, 0.5% SDS, and 50 mM Tris-Cl, pH 8). Equal amounts of protein were electrophoretically separated on a 12% SDS-PAGE gel and transferred to a PVDF membrane (Chemicon, Inc.), and specific NT-3myc immunoreactive band visualized using an alkaline phosphatase-based chemiluminescence detection kit.

Immunocytochemical analysis. SGN viability of cochlear explants was assessed quantitatively by cell counts. The whole-mount cochlear explants were fixed in 4% paraformaldehyde in 0.1 M phosphate buffer, pH 7.4 for 20 minutes and rinsed in PBS for immunocytochemistry in 96-well plates. The tissue was blocked and incubated with anti-neurofilament 200(1:500; Sigma Chemical Co.) in PBS containing 10% normal goat serum, 0.25% Triton X-100 overnight at 4° C. FITC-conjugated anti-rabbit secondary antibody (1:500; Promega) was then applied in PBS containing 10% normal goat serum, 0.25% Triton X-100 for one hour (room temperature) to reveal the labeling patterns. Only SGNs with clearly defined nuclei in each cochlea were counted by adjusting focusing planes in the Olympus epi-fluorescence microscope with a 20× lens (Leitz Orthoplan). Cells with a pyknotic or condensed nucleus were not counted.

Quantification of neurite number and statistical analysis. Neurite outgrowth in each cochlear explant was quantified using the Image-Pro quantitative analysis software (Media Cybernetics, v4.0). The image for each individual cochlear explant was captured such that a single image containing a whole cochlea, including neurites, was visible on screen. All of the tissues were viewed at 10× magnification on a Leitz Orthoplan microscope, and then the images were captured at 20× and digitized. Counts were made of the number of neurites emanating from each cochlear explant. Results presented are the means±standard error of the mean (SEM). Neurites from five cochlear explants were enumerated for each group. Data collected from each experimental group are expressed as means±SEM. Differences among means were analyzed by using a two-way analysis of variance (ANOVA). When significant differences were detected by ANOVA, a multiple comparison procedure (Student paired t test) was performed to isolate individual differences.

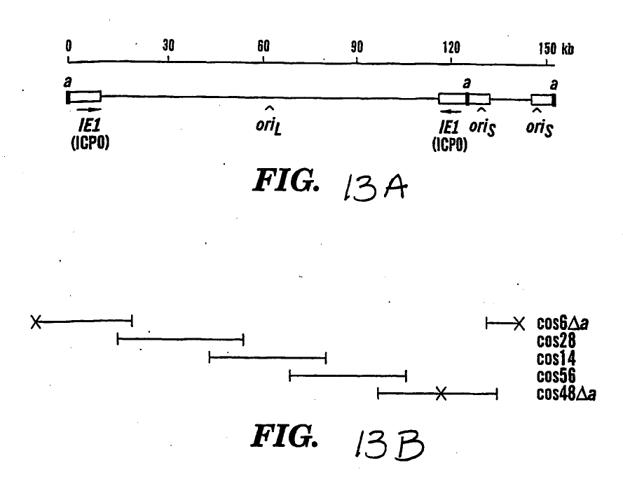

Results. To determine whether cochlear explants infected with HSVnt-3myc could produce NT-3myc RNA, RT-PCR was performed on total RNA extracted from transduced cochlear explants. The primers specific for NT-3myc gave rise to the predicted 222-bp band only in explants transduced with HSVnt-3myc (FIG. 15A, lane 2, top). NT-3myc transcripts were not observed in the control groups (FIG. 15A, lanes 1 and 3, top). A housekeeping gene, HPRT, was used as the endogenous internal control. The HPRT amplification product was the expected 416-bp size and was amplified in all culture samples (FIG. 15A, bottom). Negative control reactions that lacked reverse transcriptase during cDNA synthesis failed to yield amplification products. Furthermore, Western blot analysis was performed to assess amplicon-directed NT-3myc expression at the protein level. Protein lysates were prepared from HSVmiap—(FIG. 15B, lane 1), HSVnt-3myc—(FIG. 15B, lane 2), or mock—(FIG. 15C, lane 3) transduced cochlear explants. The myc-tagged NT-3 transgene was detected only in HSVnt-3myc-infected cultures.

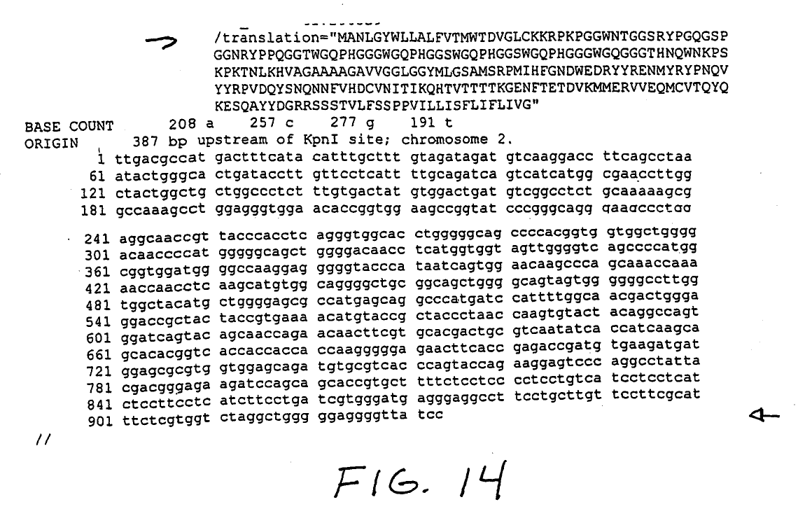

An ELISA was next utilized to determine if HSVnt-3myc transduction of cochlear explants led to secretion of NT-3 into the culture medium. Forty-eight hours following transduction with HSVnt-3myc or the control HSVmiap virus, the media were collected from transduced tissues and assayed using an NT-3-specific ELISA. As shown in FIG. 16, the mean level of NT-3 secretion from the HSVnt-3myc-transduced cochlear explants was 3161.75±137.44 pg/ml (14.43±2.84 times higher than the concentration of NT-3 contained in the control media). Endogenous NT-3 was only 213±15.66 to 219.25 pg±48.34 pg/ml in media collected from control cultures. There was a statistically significant difference between HSVnt-3myc-transduced tissues and those infected with the control virus (P<0.001) indicating that HSVnt-3myc transduction could direct cochlear explants to synthesize and secrete high levels of NT-3myc chimera

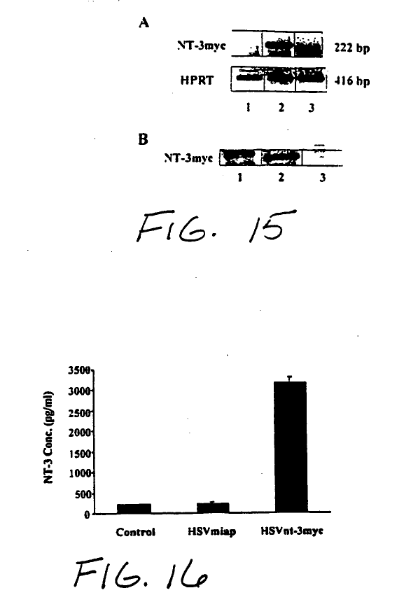

Following confirmation of NT-3myc chimera gene expression at both the RNA and the protein levels, examination of the bioactivity of the molecule was performed. Neurite outgrowth assays were utilized for this assessment. Cochlear explants were cultured in serum-free medium for 48 hours and then infected with HSVnt-3myc or HSVmiap or left uninfected. After an additional four days of culture, the extent of neurite outgrowth was assessed both qualitatively and quantitatively. Neurite density of the HSVnt-3myc-transduced group appeared significantly increased relative to the control groups as observed by immunocytochemical analysis of NF 200-positive SGNs. Amplicon-expressed NT-3myc had a dramatic, but region-specific, effect on cochlear ganglion cell density and innervation patterns. Quantitation of the number of neurites per cochlear explant for each of the treatment groups demonstrated that the enhanced neurite outgrowth was statistically different (P<0.001) (see FIG. 17).

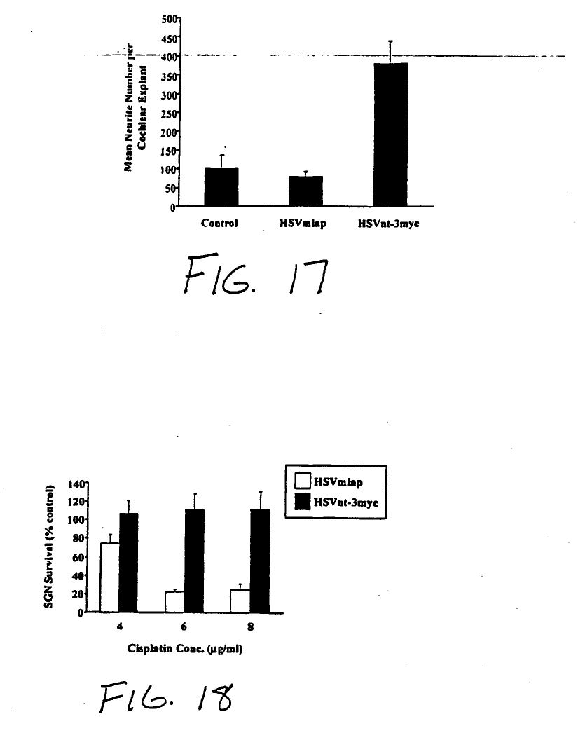

To determine whether prior HSVnt-3myc transduction could protect the SGNs from cisplatin neurotoxicity, cochlear explants were infected with HSVmiap or HSVnt-3myc for 48 hours and then treated with varying concentrations of cisplatin. Explants were subsequently immunostained with the NF 200 monoclonal antibody. When control cultures were treated at a cisplatin dosage of 6 μg/ml or higher, there were few healthy neurons that survived and the afferent fibers showed evidence of degeneration. However, overexpression of NT-3 increased the number of neurites and rescued the SGN population. When quantitation of surviving SGNs in each treatment group was performed, the percentage of SGNs surviving in HSVnt-3myc-transduced cochlear explants was significantly higher than that in the HSVmiap-transduced cultures (P<0.001) (see FIG. 18). This finding indicates that amplicon-directed NT-3myc expression protected SGNs from cisplatin neurotoxicity even at high doses of the chemotherapeutic agent.

### Example 14

### Neurotrophin-3 Transduction Attenuates Cisplatin Ototoxicity in the Aging Mouse Cochlea In Vivo

As described in the preceding example, ototoxicity is a major dose-limiting side effect of cisplatin chemotherapy for cancer patients. To address this limitation, we performed studies to demonstrated that, in vitro, HSV-1 amplicon-mediated delivery of a neurotrophin-3 (NT-3)/myc chimera protects SGNs from cisplatin-induced damage. To extend these finding, a newly constructed amplicon vector (HSVnt-3myc/SV40lac) that expresses the NT-3myc chimera and the E. coli lacZ reporter gene under separate transcriptional control was initially tested in vitro and then delivered to the cochlea of aged mice that were subsequently treated with cisplatin. Successful transduction with the new amplicon was observed in vitro as determined by its capacity to infect SGNs and to express NT-3myc mRNA and protein. To determine whether amplicon-directed NT-3 myc overexpression could abrogate the ototoxicity in vivo, two groups of aged mice (CBA) were inoculated with HSVnt-3myc/SV40lac or a control vector (HSVSV40lac) prior to administration of cisplatin. Cochleae inoculated with HSVnt-3myc/SV40lac harbored significantly greater numbers of surviving SGNs and showed lower incidence of cisplatin-induced apoptosis than those injected with the control virus. These data (which are disclosed in more detail below) demonstrate that HSV amplicon-mediated NT-3 delivery can attenuate the ototoxic actions of cisplatin in the peripheral auditory system of the aged mouse. The potency of NT-3 in SGN neuroprotection suggests utility in both chemical-induced hearing disorders and hearing degeneration due to normal aging.

Construction And Packaging Of HSV Amplicon Vectors. The PBJ-5-NT-3myc plasmid (kindly provided by Dr. Eric Shooter, Stanford University) contained a 1400-bp NT-3myc/polyA DNA fragment. To construct HSVnt-3myc/SV40lac, the SV40 promoter with blunt end from PBJ-5-NT-3myc plasmid was blunt-end cloned into a blunted SpeI site of the pHSVminOriS_{mc} amplicon vector (kindly provided by Dr. Kathleen Maguire-Zeiss, University of Rochester) to create HSVSV40lac. The CMV promoter from pHSVCMVminOris_{mc} was then subcloned into the Not I site of pHSVSV40lac amplicon vector in the opposite orientation compared to the SV40 promoter. A blunt-end fragment containing NT-3mycpolyA from PBJ-5-NT-3myc plasmid was subcloned into NsiI site (blunted) of the pHSVCMV/SV40lac vector. The HSVSV40lac amplicon served as the control vector in all experiments. Helper virus-free amplicon packaging and virus purification was performed as previously described. See Bowers et al. Gene Ther. 8:, 2001. Amplicon virus numbers were determined by assessing both expression and transduction titers as previously described. See Bowers et al. Mol. Ther. 1:294-299, 2000.

Cultures of Inner Ear Cells, Transduction of HSV Amplicon and Treatment with Cisplatin in vitro. Primary spiral ganglion neuron cultures from seven postnatal Sprague Dawley rat pups were established as previously described (Zheng et al. J. Neurosci. 15:5079-5087, 1995). The pups were sacrificed by rapid decapitation under deep halothane anesthesia and the heads were sterilized by dipping in 70% ethanol. An incision was made along the midline and the bony-cartilaginous cochlear capsule was separated from the skull. Following microdissection, the spiral ligament and stria vascularis tissue were stripped away from organ of Corti and inner ear tissue was mechanically and enzymatically dissociated with 0.25% trypsin (Sigma Chemical Co.) and 1% DNase (Sigma Chemical Co.) solution and incubated for 30 min at 37° C. The dissociated cells were plated at a density 1.5×10⁵ per well on poly-D-orithine-(Sigma Chemical Co.) coated glass coverslips in 24-well plates and maintained in DMEM/F12 media supplemented with 30 mM glucose, 2 mM glutamine, 5% horse serum, and 10% fetal calf serum. After 3 days, cultures were transduced with HSV amplicon vectors at a multiplicity of infection (MO) of 0.5 for 12 hours with a subsequent media change to remove the virus. Forty-eight hours after transduction, varying concentrations of cisplatin (0, 4, 6 and 8 μg/ml; Bristol-Myers Squibb) were added to the media for an additional 48 h of incubation.

Western Blot Analysis. Transduced primary spiral ganglion neurons were lysed in 10 mM HEPES, pH 7.5, containing 150 mM NaCl, 5 mM MgCl₂, 1 mM EGTA, 10% glycerol, 1% Triton X-100, 1 mM PMSF and protease inhibitor cocktail (Boehringer Mannheim, Indianapolis, Ind.). The protein concentration was determined using a BCA protein assay kit (Pierce, Rockford, Ill.). A total of 20 μg of protein was loaded in each lane, electrophoretically separated on a 15% SDS-polyacrylamide gel, transferred to polyvinylidene difluoride membranes (Bio-Rad), and incubated with anti-myc antibody (9E10, 1:500; Calbiochem, La Jolla, Calif.) for two hours at 22° C. as described previously. Immunoreactive protein bands were visualized using chemiluminescence-based detection (ELC kit, Amersham Pharmacia Biotech).

ELISA Assay. Conditioned media from HSVnt-3myc/SV40lac or HSVSV40lac-transduced inner ear cultures was collected 48 hours post-transduction. The level of NT-3myc secretion was quantified using a two-site immunoassay. ELISA plates (Immobilon, Nunc) were coated with anti-Human NT-3 pAb (1:500) in carbonate buffer (pH 9.7) and incubated overnight at 4° C. (NT-3 ELISA kit, Promega), followed by incubation of samples and detection of NT-3 using an anti-NT-3 mAB (1:4000) and anti-mouse IgG HRP conjugate. Data analysis was performed with at least three independent experiments. NT-3myc production was calculated using a standard curve performed on the same assay plate.

Apoptotic Nuclear Morphology Assessment. Amplicon-transduced, cisplatin-treated primary spiral ganglion neuron cultures were fixed in 4% paraformaldehyde for 20 minutes at room temperature and stained with Hoechst 33342 (1 μg/ml) for 15 minutes. The percentage of apoptotic nuclear cells in each test culture was determined by counting all cells from five random microscopic fields at 40× magnification using fluorescence microscopy.

Procedures of Surgery, Inoculation of Virus Stock and Administration of Cisplatin. Prior to surgery, CBA/CaJ aging mice (22-26 month old) were deeply anesthetized with Avertin (300 mg/kg) intraperitoneally (IP) and positioned in small animal stereotactic frame. A 5-mm diameter hole was created manually on the left bulla to expose the lateral wall of the cochlear basal turn. A small fenestrate was then made on the lateral wall of the scala vestibuli in basal turn using a specific gauge. Five μl of either HSVnt-3myc/SV40lac (2.7×10⁵ transducing unites (TU)) or HSVSV40lac (2.7×10⁵ TU) virus stock were injected into the scala vestibuli through the fenestration using a No.33, round end, Hamilton cannula connected to a 10 μl Hamilton syringe (see Suzuki et al. Gene Ther. 7:1046-54, 2000). The fenestration was sealed with a fascia of the sternocleidomastoideus muscle immediately after the administration. Two days after virus administration, the mice were treated with cisplatin (2 mg/kg/day; Bristol-Myers Squibb) by IP injection for 12 consecutive days. The animals were housed for two additional weeks prior to sacrifice, at which time tissue analysis was performed.

In situ Apoptosis Assay. A fluorescence-based apoptosis detection system was used to measure the fragmented DNA of apoptotic cells by catalytically incorporating fluorescein-12-dUTP(a) at 3-OH DNA ends using the enzyme terminal deoxynucleotidyl transferase (TdT), which forms a polymeric tail using the principle of TdT-mediated dUTP Nick-End Labeling (TUNEL; Promega) assay. The paraffin sections from each amplicon-transduced cochlea were fixed in 4% paraformaldehyde in 0.1 M-phosphate buffer (pH 7.4) for 25 minutes. at 4° C., then rinsed in PBS and permeabilized in 0.2% Triton-X-100. The samples were incubated in a solution containing the TdT enzyme at 37° C. for 60 minutes. Fluorescein-12-dUTP-labeled DNA was visualized by fluorescence microscopy. Subsets of paraffin sections were stained with propidium iodide (1 μg/ml) to visualize cellular nuclei by fluorescence microscopy. Images for each type of assay were digitally captured at a 20× magnification using a Leitz orthoplan microscope.

Toluidine Blue staining and Quantitative SGN Analysis. For quantitation of SGNs in cochleae derived from amplicon-transduced, cisplatin-treated mice, 5 μm sections were stained with toluidine blue and the number of neurons with defined cellular substructures was determined in every third section using the Image-Pro Program, V4.0, analysis software (see Zettel et al. Hear Res. 158:131-138, 2001). The image for individual samples was digitally captured and analyzed to obtain automated cell counts. All of the toluidine blue-positive cells in each section were summed in each cochlea and the total numbers were tripled. All of the sections were viewed at a 20× magnification using a Leitz orthoplan microscope. Results were expressed as the mean—standard error of the mean (SEM).

Statistical Analysis. Data collected from each experimental group were expressed as mean±SEM. Differences among means were analyzed using two-way analysis of variance (ANOVA). When significant differences were detected by ANOVA, a multiple comparison procedure (student-paired t-test method) was performed to isolate individual group differences. StataQuest 4 statistical software (State Corp., College Station, Tex.) was used for these analyses.

Results. To facilitate the monitoring of amplicon-transduced neurons in vivo, a new amplicon vector (HSVnt-3myc/SV40lac) that co-expressed the NT-3myc chimera and the surrogate marker gene β-galactosidase (lacZ) under separate transcriptional control was constructed. Reverse-transcription polymerase chain reaction (RT-PCR) and Western blot analyses were performed on transduced cochlear explants and observed expression of the NT-3myc transcript and protein only in HSVnt-3myc/SV40lac-transduced cochlear cultures as compared to cultures transduced with the control vector, HSVSV40lac. Additionally, expression of the LacZ reporter protein from the HSVnt-3myc/SV40lac vector was confirmed in transduced cochlear explants by immunocytochemistry.

The ability of the new amplicon vector to protect cultured inner ear cells from cisplatin cytotoxicity was subsequently examined. SGNs were prepared from postnatal day 3 rat pups and were transduced 3 d later with HSVnt-3myc/SV40lac or HSVSV40lac. Two days following transduction, SGN cultures were exposed to cisplatin (4, 6, or 8 g/ml) for 48 hours. After fixation and staining with Hoechst 33258, apoptotic neurons were enumerated. HSVnt-3myc/SV40lac transduction of primary SGN cultures led to a significant reduction of apoptotic cell number in cultures treated with either 4 or 6 μg/ml cisplatin as compared to companion cultures transduced with the control vector, HSVSV40lac. No protective effect was observed at the highest (8 μg/ml) dose of cisplatin.

The ability of HSV amplicon-directed NT-3myc chimera expression to protect SGNs in vivo from cisplatin-induced toxicity was subsequently evaluated in mice. Aged CBA/CaJ mice (22-26 month old) received intra-cochlear inoculations of 2.7×10⁵ transducing units of either HSVnt-3myc/SV40lac or the control vector, HSVSV40lac. Two days following virus administration, mice were treated with cisplatin for 12 consecutive days and sacrificed after an additional 14 days. Histological sections were initially stained with propidium iodide (PI) to visualize the extent of cisplatin-mediated cell loss. Sections from mice receiving HSVSV40lac and cisplatin treatment displayed qualitatively fewer PI-positive cells than those obtained from HSVnt-3myc/SV40lac pre-treated animals that had received cisplatin. This cell loss was the consequence of apoptotic cell death since TUNEL staining showed qualitatively lower numbers of positive cells in HSVnt-3myc/SV40lac-treated mice. This suggested cochlear cells undergo apoptosis in response to cisplatin and that amplicon-directed in vivo deliver of the chimeric NT-3myc protein was protective against this form ototoxicity.

Neuroprotection was demonstrated by counting toluidine blue-stained cells. Representative photomicrographs of the middle turn of the cochlear spiral were obtained from the inner ear sections prepared from amplicon and cisplatin-treated aged mice. A larger difference in the number of toluidine blue-stained cells was observed in these sections. Surviving toluidine blue-positive cells with distinct SGN morphology were enumerated. HSVnt-3myc/SV40lac-treated CBA/CaJ mice had significantly greater numbers of surviving cells than observed in HSVSV40lac-transduced animals. In aggregate, these data strongly support the hypothesis that amplicon-mediated deliver of NT-3myc provides protection against ototoxicity in vivo.

### Example 15

### Development of Integrating HSV-1 Amplicon Vectors Via Adaptation of the Sleeping Beauty Transposition System

In the studies that follow, we combined the Tc1-like Sleeping Beauty (SB) transposon system with the amplicon to engineer a novel integrating vector. Two vectors were constructed: one containing an RSV promoter-driven β-galactosidase-neomycin (βgeo) fusion flanked by the SB terminal repeats (HSVT-βgeo), and a second containing the SB transposase gene transcriptionally controlled by the HSV immediate-early 4/5 gene promoter (HSVsb). Co-transduction of BHK cells, murine primary cultures, adult striata, and neonatal brain resulted in integration of the transposable transgene (transgenon) and extension of expression duration in vivo. This new HSV amplicon iteration will protract expression profiles for gene-based amelioration of disease. We describe the methods used to conduct these studies and the results (in more detail) below.

The Sleeping Beauty transposon system. Sleeping Beauty is a synthetic transposon system that was constructed from defective units of a Tc1-like fish element. It consists of a 1.6-kb element flanked by 250-bp inverted repeats and encodes for a single protein, the Sleeping Beauty transposase. The reconstructed enzyme catalyzes transposition of ITR-flanked genetic units from one genomic locus to another. In addition, Sleeping Beauty can facilitate integration of naked DNA from episomes into human and mouse chromosomes (Ivics, 1997 #9313; Luo, 1998 #9310; Yant, 2000 #9314).

HSV amplicon particles. As noted above, the HSV amplicon is a versatile vector for gene delivery to post-mitotic cells. Because it is inherently neurotropic and easy to manipulate, the amplicon can be used to administer therapeutic agents to neurons within (or from) the central and peripheral nervous systems. Amplicons efficiently transduce mitotically active cells to achieve transient expression of proteins in vitro and in vivo. Amplicon particles made by the methods described here are particularly advantageous because they are stably maintained within cells, where they mediate long-term gene expression. Thus, expression can remain robust in dividing cell types of the CNS, such as stem-like cells or cells of the glial lineage; integration-competent viral vectors that insert into transcriptionally active chromosomal regions exhibit prolonged transgene expression profiles.

Cell culture. Baby hamster kidney (BHK) cells were maintained as described in Lu et al. (1995 #1586). The NIH-3T3 mouse fibroblast cell line was originally obtained from American Type Culture Collection and maintained in Dulbecco's modified Eagle medium (DMEM) supplemented with 10% fetal bovine serum (FBS), 100 units/ml penicillin, and 100 μg/ml streptomycin. Primary cortical neurons were harvested from E15 mice and were prepared as described by Brewer et al. (1995 #8659). Cortices were dissociated initially by trypsinization (0.25% trypsin/EDTA) for 15 min at 37° C. and washed twice with HBSS containing Ca²⁺ and Mg²⁺. Cells were mechanically dissociated further using a serologic pipette and resuspended in serum-free Neurobasal® plating medium containing 0.5 mM L-glutamine, 3.7 μg/ml L-glutamate and 2% B-27 supplement (Life Technologies, Gaithersburg, Md.). Cultures were maintained at 37° C. in a 6% CO₂ environment.

Amplicon construction. The Sleeping Beauty transposase encoding sequence was removed from the pCMV-SB plasmid ([Yant, 2000 #9314]; kindly provided by Dr. M. Kay) by XhoI-SalI digestion and cloned into the SalI site of pHSVPrPUC [Geller, 1990 #13] to create pHSVsb. The integration-competent transcription cassette from pT-βgeo [Yant, 2000 #9314] was removed using KpnI and VspI, blunted, and cloned into the blunted HindIII site of pHSVminOriSmc amplicon to create pHSVT-βgeo. In a subset of experiments, the pHSVPrPUC amplicon was employed as an empty vector control.

Helper virus-free HSV amplicon packaging. Amplicon vectors were packaged as described herein (see also Bowers, 2001 #9530]. Viral pellets were resuspended in 100 μl PBS and stored at −80° C. until use. Vectors were titered as described previously [Bowers, 2000 #9049].

Real-time Quantitative PCR Analyses. To isolate total DNA for quantitation of amplicon genomes in transduced cells or brain tissue, isolates were lysed in 100 mM potassium phosphate (pH 7.8) and 0.2% Triton X-100. An equal volumeof 2× Digestion Buffer (0.2 M NaCl, 20 mM Tris-Cl (pH 8), 50 mM EDTA, 0.5% SDS, 0.2 mg/ml proteinase K) was added to the lysate and the sample was incubated at 56° C. for four hours. Samples were processed further by one phenol:chloroform, one chloroform extraction, and a final ethanol precipitation. Total DNA was quantitated and 25 ng of total DNA was analyzed in a PE7700 quantitative PCR reaction using a designed lacZ-, or Sleeping Beauty transposase gene-specific primer/probe combination multiplexed with an 18S rRNA-specific primer/probe set.

The lacZ probe sequence was 5′-6FAM-ACCCCGTACGTCTTCCCGAGCG-TAMRA-3′; the lacZ sense primer sequence was 5′-GGGATCTGCCATTGTCAGACAT-3′; and the lacZ antisense primer sequence was 5′-TGGTGTGGGCCATAATTCAA-3′. The Sleeping Beauty probe sequence was 5′-6FAM-AAGAAGCCACTGCTCCAAAACCGACA-TAMRA-3′; the Sleeping Beauty sense primer sequence was 5′-CCACAGTAAAACGAGTCCTATATCGA-3′; and the Sleeping Beauty antisense primer sequence was 5′-TGCAAACCGTAGTCTGGCTTT-3′. The 18S rRNA probe sequence was 5′-MAX-TGCTGGCACCAGACTTGCCCTC-TAMRA-3′; the 18S sense primer sequence was 5′-CGGCTACCACATCCAAGGAA-3′; and the 18S antisense primer sequence was 5′-GCTGGAATTACCGCGGCT-3′.

Analysis of integrated vector sequences. Inverse PCR was utilized for analysis of junction fragments as previously described by Luo et al., using the identical three sets of nested primers that were designed for both the left (IR/DR-L) and right ends of the ITR (IR/DR-R) [Luo, 1998 #9310]. Briefly, genomic DNA was purified from amplicon-transduced primary neuronal cultures at Day 9 post-transduction, digested with Sau3AI, and ligated with T4 DNA ligase. Samples were subsequently subjected to three rounds of PCR using the nested primer sets. Amplified products arising from the third PCR reaction were ligated into the pGEMT-Easy cloning vector and sequenced using the dye terminator method.

Stereotactic delivery of amplicon vectors into adult mice. Eight to ten week-old male C57BL/6 mice (Jackson Laboratories) were anesthetized with Avertin (300 mg/kg) during stereotactic intrastriatal injections. After positioning in a mouse stereotactic apparatus (ASI Instruments, Warren, Mich.) the skull was exposed via a midline incision, and burr holes were drilled over the designated coordinates (Bregma, 0 mm; lateral, 2.0 mm; ventral, 3.0 mm). A 33-gauge needle was gradually advanced to the desired depth over a period of five minutes. All injections were performed with a microprocessor controlled pump (UltraMicro-Pump; WPI Instruments, Sarasota, Fla.; [Brooks, 1998 #6011]). HSVsb, HSVPrPUC, and/or HSVT-βgeo (3-6×10⁶ transduction units/ml) in 2.0 μl were injected at a constant rate over a period of five minutes (200 nl/min). Upon completion of injection, the needle was removed over a period of five minutes. Mice were sacrificed 7, 21 and 90 days post-injection for biochemical and immunocytochemical analyses.

Delivery of amplicon vectors into neonatal mice. C3H mice (P1) were anesthetized by inducing a light hyperthermia followed by manual injection of helper-free HSV amplicon virus into the right hemisphere of the brain. Specifically, a 33-gauge needle was carefully positioned above the right hemisphere and slowly advanced to the desired depth. HSVsb+HSVT-βgeo or HSVT-βgeo+HSVPrPuc in a total volume of 1 μl was manually injected. The needle was slowly removed, mice were warmed under a heat lamp and returned to their respective dams. Mice were sacrificed 90 days post-injection for immunocytochemical analyses.

Tissue preparation and immunocytochemistry. Injected adult mice were anesthetized at 7, 21, and 90 days post-injection, a catheter was placed into the left ventricle, and intracardiac perfusion was initiated with 10 ml of heparinized saline (5,000 U/L saline) followed by 60 ml of chilled 4% PFA in saline. Brains were extracted and postfixed for one to two hours in 4% PFA at 4° C. Subsequently, brains were cryoprotected in a series of sucrose solutions with a final solution consisting of a 30% sucrose concentration (w/v) in PBS. Twenty-five micron serial sections were cut on a sliding microtome (Micron/Zeiss, Thornwood, N.Y.) and stored in a cryoprotective solution (30% sucrose (w/v), 30% ethylene glycol in 0.1 M phosphate buffer (pH 7.2)) at −20° C. until processed for immunocytochemistry.

Upon removal of cryoprotectant, sections were placed into Costar net wells (VWR, Springfield, N.J.) and incubated for two hours in 0.1 M Tris buffered saline (TBS) (pH 7.6). Two additional 10 minute washes in 0.1 M TBS with 0.25% Triton X-100 (Sigma Chemical Co., St. Louis, Mo.) were performed. Sections were permeabilized in 0.1 M phosphate buffer and 0.4% Triton-X-100 for 5 minutes at 25° C. Non-specific binding sites were blocked using 0.1 M phosphate buffer, 10% normal goat serum and 0.4% Triton-X-100 for one hour at 25 ° C. Double immunolabeling was performed using anti-β-galactosidase, rabbit IgG Fraction A-11132 (1:2000, Molecular Probes, Eugene, Oreg.), with either mouse anti-Neuronal Nuclei (NeuN) monoclonal antibody (1:200, Chemicon International, Temecula, Calif.), or an anti-Glial Fibrillary Acidic Protein (GFAP)-cy3 conjugate monoclonal antibody clone G-A-5 (1:2000, Sigma, St. Louis, Mo.). Sections were incubated for 48 hours at 4° C. with primary antibodies diluted in 0.1 M phosphate buffer, 1% normal goat serum and 0.4% Triton-X-100. After rinsing in 0.1 M phosphate buffer (5×5 minutes), fluorescent secondary antibodies (fluorescein anti-rabbit IgG (H+L; 1:200, Vector Laboratories, Burlingame, Calif.), and Rhodamine Red™-X-conjugated* AffiniPure goat anti-mouse IgG (H+L) (1:200, Jackson Immuno Research Labratories Inc., West Grove, Pa.) diluted in 0.1 M phosphate buffer plus 1% normal goat serum and 0.4% Triton-X-100 were added to the sections and incubated for two hours at 25° C. The sections were rinsed in 0.1 M phosphate buffer, mounted on glass slides with Mowiol, and visualized using a confocal laser scanning microscope (FV 300, Olympus, Melville, N.Y.). All images obtained from immunocytochemical analyses were digitally acquired with a 3-chip color CCD camera at 200× magnification (DXC-9000, Sony, Montvale, N.J.).

Results. The ability of an HSV amplicon vector to deliver a transposable transcription unit for preferential expression in cells of glial origin was examined using a two-vector approach. One amplicon was constructed to express high levels of the Sleeping Beauty transposase (HSVsb) under transcriptional control of the HSV immediate-early 4/5 promoter. The second amplicon served as the substrate vector for the transposase and carried a terminal inverted repeat-flanked transgene segment (termed ‘transgenon’) which expressed a β-galactosidase-neomycin resistance gene fusion under Rous sarcoma virus (RSV) long terminal repeat transcriptional control (HSVT-βgeo). This promoter is widely expressed, but when employed in the context of the CNS imparts expression selectivity to specific regions of the brain [Smith, 2000 #9727]. We employed a two-vector strategy since inclusion of both components in one vector would likely lead to transposition events occurring within the packaging cell resulting in inefficient virion generation. The two vectors were packaged separately using a modified helper virus-free method [Bowers, 2001 #9530].

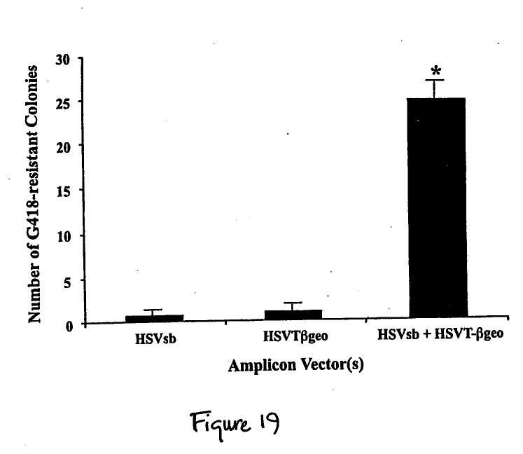

To determine if co-transduction with two amplicon vectors would result in enhanced integration in mitotically active cells, we initiated testing in baby hamster kidney (BHK) cells. BHK cultures were transduced with equivalent virion numbers of HSVsb+HSVPrPUC (empty vector control), HSVT-βgeo+HSVPrPUC, or HSVT-βgeo+HSVsb. Cultures subsequently were placed under G418 selection, and resistant colonies that arose following two weeks of drug selection were stained by X-gal histochemistry and enumerated. Co-transduction of HSVsb or HSVT-βgeo with the empty vector control amplicon resulted in very few numbers of G418-resistant, LacZ⁺ colonies (FIG. 19).

However, co-transduction of HSVsb with HSVT-βgeo greatly increased the numbers of colonies (˜25-fold), indicating that an HSV amplicon-harbored transgenon could be stably maintained and expressed only when briefly exposed to the transposase expressed from HSVsb. The expression kinetics of HSVsb was not measured directly, but based upon previous work with other transgenes expressed from the HSVPrPUC backbone, expression levels are highest at 24-48 hours post-transduction and wane over the succeeding 10 days ([Jin, 1996 #4659]).

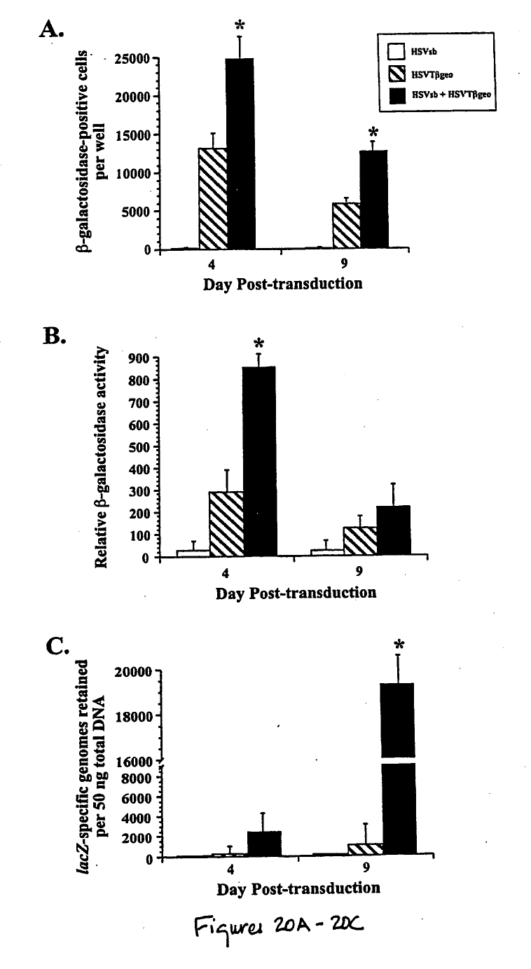

The observations made in actively dividing BHK cells led us to test the new bipartite amplicon platform in primary murine cortical cultures to determine if transposition of the amplicon-bearing transgene unit could occur in cells within the central nervous system. Demonstration of such an event and examination of resultant expression duration profiles and cellular specificity would lead to the design of novel HSV amplicons for treatment of neurodegenerative diseases. Primary cultures were established using B27 medium in the absence of mitotic inhibitors, which has been shown to provide cultures consisting of mainly neuronal cell types with minimal glial contamination. As time in culture increases the population of glial cells is gradually amplified. Primary cultures were established from cortices of embryonic day 15 (E15) C57BL/6 embryos and incubated with equivalent transducing virion numbers of HSVsb, HSVT-βgeo, or both vectors on in vitro day 5 (DIV 5). Treated cultures were processed for X-gal histochemistry, β-galactosidase enzyme activity, and real-time quantitative PCR analysis of the transgenon DNA segment on Days 4 and 9 post-transduction. Enumeration of X-gal-positive cells in each of the treatment groups indicated that cultures receiving both test amplicons exhibited enhanced numbers of transgene-expressing cells on Days 4 and 9 (FIG. 20A). Separate immunocytochemical analysis of cultures indicated that both neurons and glia expressed the βgeo transgene. Analysis of transgene-encoded β-galactosidase enzyme activity by Galacto-Lite™ assay exhibited similar profiles of expression between the three treatment groups on Day 4 but differences in β-galactosidase activity did not reach statistical significance at Day 9 among the groups (FIG. 20B). Interestingly, when total DNA was harvested from transduced cultures using a method favoring the purification of larger molecular weight DNA, the cultures receiving both test amplicons exhibited an increased number of lacZ sequence targets over time as detected by real-time quantitative PCR (FIG. 20C). These results in aggregate suggested that the transgenon segment of the HSVT-βgeo amplicon had mobilized into the host cell genome in an HSVsb-dependent manner that resulted in appreciably enhanced gene expression as compared to HSVT-βgeo alone.

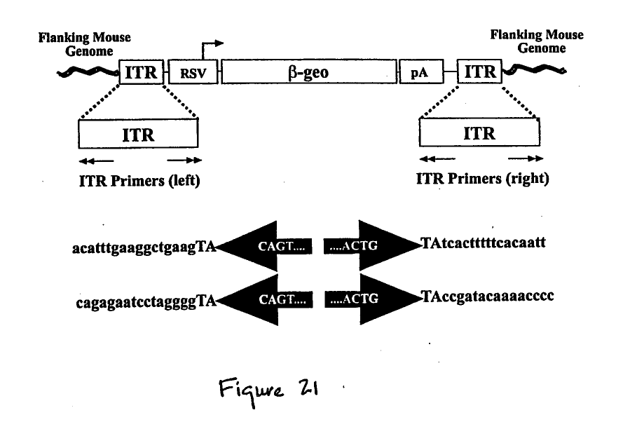

To definitively assess the occurrence of Sleeping Beauty-mediated integration in mouse primary culture cells, we employed inverse PCR as previously described by Luo and colleagues [Luo, 1998 #9310]. On Day 9 post-transduction, high molecular weight DNA isolated from primary cultures that had been treated with both HSVsb and HSVT-βgeo was subjected to three rounds of nested PCR. Resultant integration junction PCR products were sequenced and analyzed for identity of novel flanking nucleotide sequences. We were able to identify several different flanking sequences that corresponded to murine genomic sequence as assessed from BLAST searches (FIG. 21). There did not appear to be a preference for particular integration sites within the genome as determined by the analysis of numerous inverse PCR products.

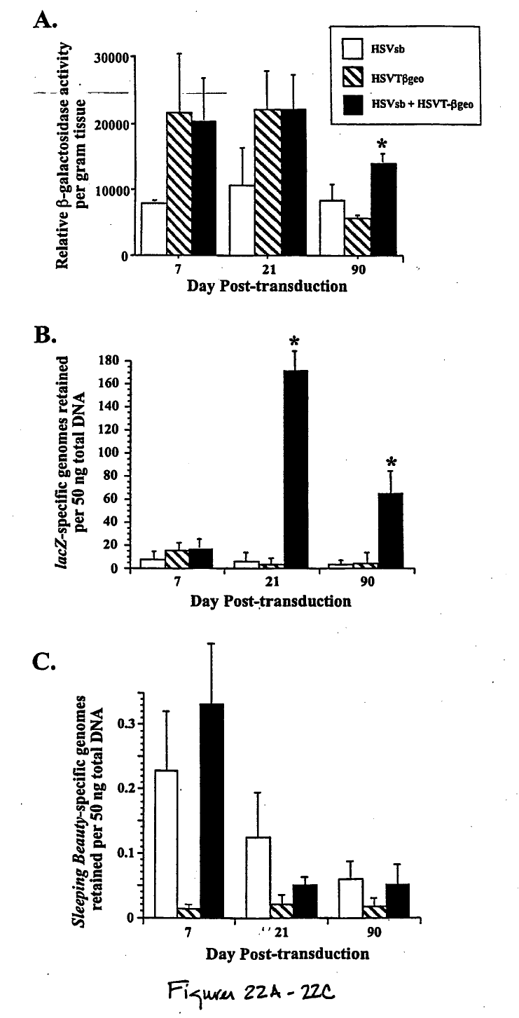

We subsequently characterized the new integration-competent amplicon vector platform in vivo in the setting of the murine CNS. Three month-old male C57BL/6 mice were transduced with equivalent virion numbers of HSVsb, HSVT-βgeo, or both vectors and were processed for β-galactosidase enzyme activity, real-time quantitative PCR analyses, and immunocytochemistry on Days 7, 21, and 90 post-transduction. The empty vector, HSVPrPUC, was used in the single vector treatments for equilibration of virus particle input. The temporal expression pattern of β-galactosidase was indistinguishable for animals receiving HSVT-βgeo alone and those receiving HSVsb plus HSVT-βgeo on Days 7 and 21 post-transduction (FIG. 22A). At Day 90, there existed a statistically significant difference in transgene expression levels between these two groups as well as the HSVsb-transduced mice. When transgene DNA retention analyses were performed on high molecular weight nucleic acid purified from the injection site, we detected greatly enhanced numbers of transgenon-specific sequences only in animals receiving both HSVsb and HSVT-βgeo amplicons (FIG. 22B). To confirm that only the T-βgeo transgenon segment co-segregated with genomic DNA, we performed quantitative real-time PCR for Sleeping Beauty transposase gene sequences that are harbored in the HSVsb amplicon. The transposase-specific sequences were readily detectable on Day 7 but were difficult to detect above background signals on Days 21 and 90 post-transduction, indicating that transposition events were specific to the transgenon-carrying amplicon vector, HSVT-βgeo (FIG. 22C). As stated above, in vivo amplicon administration was performed using equal virion numbers of HSVsb and HSVT-βgeo (or the control HSVPrPUC amplicon, where appropriate).

The in vivo biochemical data suggested that cells of the murine CNS were amenable to transposition of a mobilization-competent transcription unit from an amplicon into the cellular genome. To identify the cell type(s) harboring and expressing the transgenon we utilized fluorescent immunocytochemistry to visualize lacZ in conjunction with the neuronal marker, NeuN, or the glial cell marker, GFAP. Transgenon-derived β-galactosidase expression consistently localized to GFAP-positive cells in mice receiving HSVT-βgeo or the HSVsb/HSVT-βgeo combination, and was rarely, if ever, detected in NeuN-positive neurons (n=12). Differences in transgene expression duration existed amongst the various treatment groups. We were able to detect lacZ expression only in brains receiving the combined HSVsb/HSVT-βgeo amplicon treatment at Day 90 post-transduction, further confirming the results obtained from enzyme activity assays (FIG. 22A). Performance of titration studies by varying either amplicon component did not alter cell type specificity of transgenon expression.

To examine the potential applicability of the new integrating system in perinatal gene transfer paradigms for therapeutic applications or the creation of novel degenerative disease models, we administered the new amplicon vector platform to the CNS of newborn mice. One day-old (P0) C3H mice were transduced with HSVT-βgeo alone, or both HSVsb and HSVT-βgeo and were processed for fluorescent immunocytochemistry on Day 90 post-transduction. As with the adult C57BL/6 animals, striata from C3H mice transduced at P0 exhibited lacZ transgene expression only in GFAP-positive cells. Transgenon expression at Day 90 was dependent upon co-transduction of the HSVsb and HSVT-βgeo amplicons, as animals receiving only HSVT-βgeo did not exhibit any detectable lacZ expression at this time point. In aggregate, these results indicate that this new integrating HSV amplicon vector system extends the utility of this gene delivery platform to provide prolonged transgene expression within cells of the CNS that were once refractory to stable amplicon-mediated expression.

1. A method of generating a herpes virus amplicon particle, the method comprising
providing a cell that has been stably transfected with a nucleic acid sequence that encodes an accessory protein; and
transfecting the cell with (a) one or more packaging vectors that, individually or collectively, encode one or more HSV structural proteins but do not encode a functional herpes virus cleavage/packaging site (b) an amplicon plasmid comprising a sequence that encodes a functional herpes virus cleavage/packaging site, a herpes virus origin of DNA replication, and a heterologous transgene and (c) an integration vector, wherein the integration vector encodes an enzyme that catalyzes a reaction within the cell, the consequence of the reaction being that the transgene carried by the amplicon plasmid is inserted into the genome of the cell.

2. (canceled)

3. A method of generating a herpes virus amplicon particle, the method comprising cotransfecting a host cell with the following:
(a) an amplicon plasmid comprising an HSV origin of replication, an HSV cleavage/packaging signal, and a heterologous transgene expressible in the host cell;
(b) one or more packaging vectors that, individually or collectively, encode all essential HSV genes but exclude all cleavage/packaging signals;
(c) a vector encoding an accessory protein; and
(d) an integration vector, wherein the integration vector encodes an enzyme that catalyzes a reaction within the cell, the consequence of the reaction being that the transgene carried by the amplicon plasmid is inserted into the genome of the cell.

4. (canceled)

5. A method of generating a herpes virus amplicon particle, the method comprising transfecting a cell with
(a) one or more packaging vectors that, individually or collectively, encode one or more HSV structural proteins but do not encode a functional herpes virus cleavage/packaging site;
(b) an amplicon plasmid comprising a sequence that encodes a functional herpes virus cleavage/packaging site, a herpes virus origin of DNA replication, and a transgene that encodes a prion protein, an antigenic fragment thereof, or a single chain antibody that specifically binds a prion protein; and
(c) a nucleic acid sequence that encodes an accessory protein.

6. (canceled)

7. The method of claim 1, wherein the herpes virus is an alpha herpes virus or an Epstein-Barr virus.

8. The method of claim 7, wherein the alpha herpes virus is a Varicella-Zoster virus, a pseudorabies virus, or a herpes simplex virus.

9. The method of claim 1, wherein the accessory protein inhibits the expression of a gene in the cell.

10. The method of claim 1, wherein the accessory protein is a virion host shutoff protein.

11. The method of claim 10, wherein the virion host shutoff protein is an HSV-1 virion host shutoff protein, an HSV-2 virion host shutoff protein, an HSV-3 virion host shutoff protein, bovine herpes virus 1 virion host shutoff protein, bovine herpes virus 1.1 virion host shutoff protein, gallid herpes virus 1 virion host shutoff protein, gallid herpes virus 2 virion host shutoff protein, suid herpes virus 1 virion host shutoff protein, baboon herpes virus 2 virion host shutoff protein, pseudorabies virus virion host shutoff protein, cercopithecine herpes virus 7 virion host shutoff protein, meleagrid herpes virus 1 virion host shutoff protein, equine herpes virus 1 virion host shutoff protein, or equine herpes virus 4 virion host shutoff protein.

12. The method of claim 10, wherein the virion host shutoff protein is operatively coupled to its native transcriptional control elements.

13. The method of claim 1, wherein the cell is further transfected with a sequence encoding a VP16 protein, wherein the VP16 protein is transiently or stably expressed.

14. The method of claim 13, wherein the VP16 protein is HSV1 VP16, HSV-2 VP16, bovine herpes virus 1 VP16, bovine herpes virus 1.1 VP 16, gallid herpes virus 1 VP16, gallid herpes virus 2 VP16, meleagrid herpes virus 1 VP16, or equine herpes virus 4 VP16.

15. The method of claim 1, wherein the one or more packaging vectors comprises a cosmid, a yeast artificial chromosome, a bacterial artificial chromosome, a human artificial chromosome, or an F element plasmid.

16. The method of claim 1, wherein the one or more packaging vectors comprises a set of cosmids comprising cos6Δa, cos28, cos14, cos56, and cos48Δa.

17. The method of claim 1, wherein the one or more packaging vectors, individually or collectively, express the structural herpes virus proteins.

18. The method of claim 1, wherein the transgene encodes a therapeutic protein or RNA molecule.

19. The method of claim 18, wherein the therapeutic RNA molecule is an antisense RNA molecule, siRNA, or a ribozyme.

20. The method of claim 18, wherein the therapeutic protein is a receptor, a signaling molecule, a transcription factor, a growth factor, an apoptosis inhibitor, an apoptosis promoter, a DNA replication factor, an enzyme, a structural protein, a neural protein, or a histone.

21. The method of claim 18, wherein the therapeutic protein is an immunomodulatory protein, a tumor-specific antigen, or an antigen of an infectious agent.

22. The method of claim 21, wherein the immunomodulatory protein is a cytokine or a costimulatory molecule.

23. The method of claim 22, wherein the cytokine is an interleukin, an interferon, or a chemokine.

24. The method of claim 22, wherein the costimulatory molecule is a B7 molecule or CD40L.

25. The method of claim 21, wherein the tumor-specific antigen is a prostate specific antigen.

26. The method of claim 21, wherein the infectious agent is a virus or a prion protein.

27. The method of claim 26, wherein the virus is a human immunodeficiency virus.

28. The method of claim 23, wherein the antigen of an infectious agent is gp120.

29. The method of claim 21, wherein the infectious agent is a bacterium or parasite.

30. The method of claim 1, wherein the amplicon plasmid further comprises a promoter.

31-65. (canceled)


SEQUENCE LISTING (24 sequences)

SEQ ID NO: 1 - protein, 10 aa, Human immunodeficiency virus
RGPGRAFVTI

SEQ ID NO: 2 - DNA, 35 bp, Artificial Sequence
  Synthetically generated oligonucleotide
cggaattccgcaggttttgtaatgtatgtgctcgt

SEQ ID NO: 3 - DNA, 38 bp, Artificial Sequence
  Synthetically generated oligonucleotide
ctccgaagcttaagcccgatatcgtctttcccgtatca

SEQ ID NO: 4 - DNA, 22 bp, Artificial Sequence
  Probe sequence
accccgtacgtcttcccgagcg

SEQ ID NO: 5 - DNA, 22 bp, Artificial Sequence
  Primer
gggatctgccattgtcagacat

SEQ ID NO: 6 - DNA, 22 bp, Artificial Sequence
  Probe sequence
tgctggcaccagacttgccctc

SEQ ID NO: 7 - DNA, 20 bp, Artificial Sequence
  Primer
cggctaccacatccaaggaa

SEQ ID NO: 8 - DNA, 18 bp, Artificial Sequence
  Primer
gctggaattaccgcggct

SEQ ID NO: 9 - DNA, 22 bp, Artificial Sequence
  Probe sequence
caggaccacttctgcgctcggc

SEQ ID NO: 10 - DNA, 21 bp, Artificial Sequence
  Primer
ctggatggaggcggataaagt

SEQ ID NO: 11 - DNA, 22 bp, Artificial Sequence
  Primer
tgctggcaccagacttgccctc

SEQ ID NO: 12 - DNA, 22 bp, Artificial Sequence
  Probe sequence
tgctggcaccagacttgccctc

SEQ ID NO: 13 - DNA, 20 bp, Artificial Sequence
  Primer
cggctaccacatccaaggaa

SEQ ID NO: 14 - DNA, 18 bp, Artificial Sequence
  Primer
gctggaattaccgcggct

SEQ ID NO: 15 - DNA, 20 bp, Artificial Sequence
  Primer
tggtgtgggccataattcaa

SEQ ID NO: 16 - DNA, 22 bp, Artificial Sequence
  Primer
atgaaacgaggtgtaaagaagc

SEQ ID NO: 17 - DNA, 20 bp, Artificial Sequence
  Primer
ctgatgagcttctgctcgcc

SEQ ID NO: 18 - DNA, 21 bp, Artificial Sequence
  Primer
ctgacctgctggattacatta

SEQ ID NO: 19 - DNA, 21 bp, Artificial Sequence
  Primer
ccactttcgctgatgacacaa

SEQ ID NO: 20 - DNA, 26 bp, Artificial Sequence
  Probe sequence
aagaagccactgctccaaaaccgaca

SEQ ID NO: 21 - DNA, 26 bp, Artificial Sequence
  Primer
ccacagtaaaacgagtcctatatcga

SEQ ID NO: 22 - DNA, 21 bp, Artificial Sequence
  Primer
tgcaaaccgtagtctggcttt

SEQ ID NO: 23 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 24 - DNA, 933 bp, Mus musculus
ttgacgccatgactttcatacatttgctttgtagatagatgtcaaggaccttcagcctaaatactgggcactgataccttgttcctcattttgcagatcagtcatcatggcgaaccttggctactggctgctggccctctttgtgactatgtggactgatgtcggcctctgcaaaaagcggccaaagcctggagggtggaacaccggtggaagccggtatcccgggcagggaagccctggaggcaaccgttacccacctcagggtggcacctgggggcagccccacggtggtggctggggacaaccccatgggggcagctggggacaacctcatggtggtagttggggtcagccccatggcggtggatggggccaaggagggggtacccataatcagtggaacaagcccagcaaaccaaaaaccaacctcaagcatgtggcaggggctgcggcagctggggcagtagtggggggccttggtggctacatgctggggagcgccatgagcaggcccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatccagcagcaccgtgcttttctcctcccctcctgtcatcctcctcatctccttcctcatcttcctgatcgtgggatgagggaggccttcctgcttgttccttcgcatttctcgtggtctaggctgggggaggggttatcc